(19) United States
(12) Patent Application Publication
(10) Pub. No.: US 2025/0333695 A1
(43) Pub. Date: Oct. 30, 2025

(54) METHODS FOR OPTIMIZING T CELL IMMUNOTHERAPEUTIC EFFECTOR AND MEMORY FUNCTION

(71) Applicant: THE TRUSTEES OF THE UNIVERSITY OF PENNSYLVANIA, Philadelphia, PA (US)

(72) Inventors: Christoph T. Ellebrecht, Philadelphia, PA (US); Aimee S. Payne, Merion Station, PA (US); Casey S. Lee, Philadelphia, PA (US)

(21) Appl. No.: 18/866,260

(22) PCT Filed: May 16, 2023
(86) PCT No.: PCT/US2023/067063

§ 371 Date: Nov. 15, 2024

Related U.S. Application Data
(60) Provisional application No. 63/343,006, filed on May 17, 2022.

(51) Int. Cl. C12N 5/0783; A61K 35/17; A61K 40/11; A61K 40/31; C12N 5/00; C12N 11/16; G01N 33/50

(52) U.S. Cl. CPC C12N 5/0636; A61K 35/17; A61K 40/11; A61K 40/31; C12N 5/0087; C12N 11/16; C12Y 304/2207; G01N 33/5005

(57) ABSTRACT

The present disclosure provides methods for identifying and enriching proximal and/or distal daughter CAR T cells, and methods for use thereof, including treatment of diseases, such as cancer.

### CROSS-REFERENCE TO RELATED APPLICATION

The present application is entitled to priority under 35 U.S.C. § 119(e) to U.S. Provisional Patent Application No. 63/343,006, filed May 17, 2022, which is hereby incorporated by reference in its entirety herein.

### STATEMENT REGARDING FEDERALLY SPONSORED RESEARCH OR DEVELOPMENT

This invention was made with government support under AR080266 awarded by the National Institutes of Health. The government has certain rights in the invention.

### SUMMARY OF THE INVENTION

In one aspect, the disclosure provides a method of enriching, from a population of CAR T cells, a distal first division daughter chimeric antigen receptor (CAR) T cell or population thereof. The method comprises measuring a set of genes expressed in each of the CAR T cells in the population, wherein the genes are selected from the group consisting of CD103 (Integrin alpha E), CD45RA, CD99.1, TCRalpha/beta, CD101 (BB27), CD8, CD49a, CD7.1, CD48.1, CD52.1, CD73, CD5.1, CD3, CD224, CD45, CD47.1, CD11a, CD18, CD31, CD27.1, mouse CD49f, CD2.1, CD26, CD195 (CCR5), CD38.1, CD244 (2B4), CD29, CD314 (NKG2D), CD305 (LAIR1), CD352 (NTB-A), CD49d, CD95Fas, CD69.1, integrin beta 7, CD44.1, CD273B7 (DCPD-L2), CD45RO, CD96 (TACTILE), CD57 Recombinant, CD94, CD56, CD49b, CD226 (DNAM-1), CD278 (ICOS), CD127 (IL-7R), CLEC12A.1, CD39, CD161, HLA-DR, CD81 (TAPA-1), HLA-E.1, HLA-ABC, CD66ace, and CD28.1, wherein when expression of at least one of these genes is increased in the CAR T cell relative to a control, the cell is identified as a distal daughter CAR T cell and is collected.

In certain embodiments, the control is selected from the group consisting of a proximal daughter CAR T cell, a resting CAR T cell, and a non-enriched CAR T cell population.

Another aspect of the disclosure includes a composition comprising a population of distal daughter CAR T cells isolated by any of the methods contemplated herein.

In certain embodiments, the population comprises over 50%, 60%, 70%, 80%, 90%, or 100% distal daughter CAR T cells.

Another aspect of the disclosure includes a method of enriching, from a population of CAR T cells, a proximal first division daughter CAR T cell or population thereof. The method comprises measuring a set of genes expressed in each of the CAR T cells in the population, wherein the genes are selected from the group consisting of CD30, CD71, CD25, CD106, CD117 (c-kit), CD88 (C5aR), CD11c, CD155 (PVR), CD223 (LAG-3), CD146, CD163.1, CD19.1, CD194 (CCR4), CD23, Notch1, CD105, CD169 (Sialoadhesin Siglec-1), CD83.1, aCD207, CD137 (4-1BB), TCRdeltagamma, CD134 (OX40), B7-H4, CD324E (Cadherin), CD122 (IL-2R beta), CD10, CD206 (MMR), CD178 (Fas-L), CD82.1, CD141 (Thrombomodulin), CD80.1, LOX-1, CD1a, IgGFc, CD123, TCRValpha24, CD209 (DC-SIGN), CD272 (BTLA), CD304 (Neuropilin-1), CD85 (jILT2), CD252 (OX40L), CD303 (BDCA2), IgM, TSLPRTSLP-R, CD98, CD34.1, CD20, CD235ab, CD62L, CD144VE (Cadherin), CD307d (FcRL4), CD197 (CCR7), CD201 (EPCR), CD54, CX3CR1.1, CD360 (IL-21R), CD140b (PDGFRbeta), CD112 (Nectin-2), CD124 (IL-4Ralpha), CD257 (BAFFBLYS), CD335 (NKp46), CD152 (CTLA-4), EGFR.1, GARPLRRC32, CD62E, CD269 (BCMA), CD158 (KIR2DLISIS3S5), Podoplanin, XCR1.1, CD70.1, CD254 (TRANCERANKL), Podocalyxin, CD158f (KIR2DL5), CD274 (B7-H1 PD-L1), CD66b, CD21, CD307 (eFcRL5), CD268 (BAFF-R), CD154, CD137L (4-1BB Ligand), CD119 (IFN gamma R alpha chain), CD1d, CD370 (CLEC9ADNGR1), CD267 (TACI), CD107a (LAMP-1), CD24.1, CD13, TCRVgamma9, CD357 (GITR), TCRVdelta2, Notch3, CD40.1, CD326 (Ep-CAM), CD204, Fc epsilon RI alpha, CD294 (CRTH2), CD158e1 (KIR3DL1, NKB1), CD150 (SLAM), CD14.1, Ig light chain kappa, LIPSTIC1, CD184 (CXCR4), CD196 (CCR6), CD79b (IgB), CD16, DR3TRAMP, CD319 (CRACC), CD258 (LIGHT), CD32, TCRVbeta131, CD275 (B7-H2, ICOSL), CD45R/B220, CD279, CD35, CD42b, CD366 (Tim-3), CD336 (NKp44), CD140a (PDGFRalpha), IgD, CD62P (P-Selectin), CD1c, CD41, CD11b, CD185 (CXCR5), CD22.1, CD328 (Siglec-7), CD325 (N-Cadherin), CD86.1, CD36.1, CD158b (KIR2DL2/L3, NKAT2, KLRG1, MAFA), Ig light chain lambda, GPR56, TIGIT/VSTM3, CD337 (NKp30), CD64, CD33.1, TCRValpha72, CD270 (HVEMTR2), CD4.1, HLAA2, CD183 (CXCR3), and CD58 (LFA-3), wherein when expression of at least one of these genes is increased relative to a control, the cell is identified as a proximal daughter CAR T cell and is collected.

In certain embodiments, the control is selected from the group consisting of a distal daughter CAR T cell, a resting CAR T cell, or a non-enriched CAR T cell population.

Another aspect of the disclosure includes a composition comprising a population of proximal daughter CAR T cells isolated by any of the methods contemplated herein.

In certain embodiments, the population comprises over 50%, 60%, 70%, 80%, 90%, or 100% proximal daughter CAR T cells.

Another aspect of the disclosure includes a method of enriching, from a population of CAR T cells, a distal first division daughter CAR T cell or population thereof. The method comprises: i) pre-loading a LPETG peptide comprising a detectable label onto a Sortase A (SrtA) molecule, ii) fusing the SrtA to a target protein on a target cell, iii) labeling with a dye, a CAR T cell comprising at least one N-terminal glycine on the CAR, iv) incubating the target cell with the labeled CAR T cell, v) assessing CAR T cell division by dye dilution, indicating daughter cell formation, and vi) measuring the detectable label on the daughter cells following the first cell division of the CAR T cell, wherein when the detectable label is present, the cell is a proximal first division daughter cell, and wherein when the detectable label is absent, the cell is a distal first division daughter cell, and collecting the distal daughter CAR T cell.

In certain embodiments, the target protein is a tumor associated antigen (TAA).

In certain embodiments target cell is selected from the group consisting of a cancer cell, an autoimmune cell, an alloimmune immune cell, an infected cell, and a diseased cell in a fibrotic disease.

In certain embodiments the detectable label is a biotin or a fluorophore.

In certain embodiments the dye is selected from the group consisting of CFSE, CellTrace™ Violet, CellTrace™ Red, and CellTrace™ Yellow.

In certain embodiments, the method further comprises allowing the daughter cell to undergo a second division, and isolating and/or collecting the distal second division daughter cell.

In certain embodiments, the method further comprises allowing the daughter cell to undergo a third division, and isolating and/or collecting the distal third division daughter cell.

Another aspect of the disclosure includes a composition comprising a population of distal second division daughter cells isolated and/or collected by any of the methods contemplated herein.

Another aspect of the disclosure includes a composition comprising a population of distal third division daughter cells isolated and/or collected by any of the methods contemplated herein.

Another aspect of the disclosure includes a composition comprising a population of first and/or second, and/or third division daughter cells isolated and/or collected by any of the methods contemplated herein.

Another aspect of the disclosure includes a method of inducing a T cell to adopt a distal first division daughter cell phenotype. The method comprises introducing at least one transcription factor into a primary T cell such that the transcription factor is transiently overexpressed, wherein the transcription factor is selected from the group consisting of STAT1, STAT2, KDM5B, MXD4, MAX, KLF2, FLI1, IRF2, IRF7, IRF3, IRF9, ELK1, ELK4, ZNF358, and IKZF1.

In certain embodiments, the method further comprises isolating and/or collecting the distal first division daughter cell, or population thereof.

In certain embodiments, the T cell is a chimeric antigen receptor (CAR) T cell.

In certain embodiments, the method improves the efficacy and longevity of the CAR T cell.

In certain embodiments, the method further comprises isolating the distal first division daughter CAR T cell, or population thereof.

Another aspect of the disclosure includes a method of inducing a T cell to adopt a proximal first division daughter cell phenotype. The method comprises introducing at least one transcription factor into a primary T cell such that the transcription factor is transiently overexpressed, wherein the transcription factor is selected from the group consisting of MYC, TP73, MYBL1, SP2, YBX1, E2F2, E2F7, E2F8, and NFYB.

In certain embodiments, the method further comprises isolating and/or collecting the proximal first division daughter cell, or population thereof.

In certain embodiments, the T cell is a chimeric antigen receptor (CAR) T cell.

In certain embodiments, the method further comprises isolating the proximal first division daughter CAR T cell, or population thereof.

In certain embodiments, the transcription factor is introduced via a method selected from the group consisting of electroporation of the protein, mRNA, or circular RNA form of the transcription factor.

In certain embodiments, the transcription factor is introduced as an mRNA or circular RNA encapsulated in a lipid nanoparticle (LNP).

In certain embodiments, a single transcription factor is introduced into the cell.

In certain embodiments, a plurality of transcription factors are introduced into the cell.

Another aspect of the disclosure includes a method of enriching for distal daughter CAR T cells in a population of CAR T cells, the method comprising stimulating a CAR T cell with a target cell and collecting the CAR T cell progeny 1 day, 2 days, 3 days, 4 days, 5 days, 6 days, or 7 days after stimulation, wherein the progeny is thereby enriched for distal daughter CAR T cells.

Another aspect of the disclosure includes a method of treating a disease or disorder. The method comprises administering to a subject in need thereof, any one of the compositions contemplated herein.

### BACKGROUND OF THE INVENTION

Chimeric antigen receptor T (CART) cell therapy comprises T cells with both effector (cytolytic) as well as memory function. Long-term persistence of chimeric antigen receptor T (CART) cells is associated with superior outcome and credited to the formation of long-lived memory CART cells that afford continuous immunosurveillance. Despite clinical efficacy of CART therapy in some patients, a substantive proportion of patients either do not achieve long-term remission or relapse after CART therapy. There is thus an unmet need to understand cellular mechanisms of effector and memory T cell formation in cellular immunotherapy applications and optimize therapeutic approaches accordingly. The present embodiments address this need as well as others.

### BRIEF DESCRIPTION OF THE DRAWINGS

The following detailed description of specific embodiments will be better understood when read in conjunction with the appended drawings. For the purpose of illustrating the embodiments, there are shown in the drawings exemplary embodiments. It should be understood, however, that the embodiments are not limited to the precise arrangements and instrumentalities of the embodiments shown in the drawings.

FIG. 1: Labeling Immune Partnerships by SorTagging Intercellular Contacts (LIPSTIC) allows distinction of proximal and distal 1^{st} division daughter CAR T cells. Left panel: Target cells express the CAR target protein with a tethered sortase mutant that transfers the sortase substrate LPETG peptide to the n-terminal pentaglycine residue of the CAR only after the CAR comes into proximity with the sortase (i.e. after the CAR binds to the target protein). Middle panels: Activated CAR T cells have a mixture of labeled and unlabeled CAR molecules on their surface and undergo the first cell division after activation. Since the immunological synapse (including the labeled CAR molecules) is inherited by the proximal daughter cell (i.e. the cell that had contact with the target), the LPETG (aka LIPSTIC) label is only present on the proximal daughter cell and allows distinction of proximal and distal first division daughter cells (right panel).

FIG. 2: LIPSTIC allows distinction of proximal and distal 1^{st} division daughter CAR T cells. Target cells: Nalm6 cells with CD19 knockout (KO) and subsequently engineered to express CD19-sortase; and Nalm6 cells engineered to express gdTCR-sortase. CARs: Minimal modification to add n-terminal pentaglycine tag between the signal peptide and ScFV, CART19 (bbz, 28z), CARTdelta (bbz,28z). Right panel: Shown is an example of the insertion of five glycine residues between the signal peptide and the antibody fragment (single chain variable fragment or ScFV) of the CAR. Upper row: DNA sequence with exemplary restriction sites, bottom row: amino acid sequence. N-terminal refers to the mature CAR molecule on the cell surface, i.e. after cleavage of the signal peptide.

FIGS. 3A-3B: LIPSTIC+ and LIPSTIC− 1^{st} division daughter cells demonstrate distinct phenotypes, confirming asymmetric cell division in human CAR T cells. Gated on live, GFP− cells. LIPSTIC+ and LIPSTIC− daughter cells demonstrate differences in: size, proliferation pace, CD25 expression. This result was reproducible for CAR T cell targeting different antigens (e.g. CD19, gamma-delta TCR). FIG. 3A: CAR T cells were labeled with CellTrace Violet as described in materials and methods. Target cells were labeled with biotin-LPETG (=LIPSTIC) peptide and AF647-streptavidin as detailed in materials and methods. CAR T cells and target cells were co-incubated for the indicated times and analyzed by flow cytometry. Unstimulated=CAR T cells cultured without target cells; Unspecific activation=CAR T cells activated with anti-CD3/anti-CD28 beads; Specific activation=CAR T cells activated with NALM6 target cells labeled with LPETG peptide. Top gate=proximal daughter cells, bottom gate=distal daughter cells. Bottom row of panel A: Histograms corresponding to the scatter plots shown in the top row. Numbers adjacent to peaks refer to number of cell divisions. FIG. 3B: Sorted proximal, distal and unstimulated CAR T cells were analyzed by flow cytometry after 72 hours in culture. Shown are plots comparing cell size (left), numbers of cell division (middle panel), and CD25 positivity (right panel) 72 hours following sorting of proximal, distal, and unstimulated CAR T cells. Cells were stained with an anti-human CD25 antibody for 20 minutes at room temperature and washed twice prior to analysis on a BD LSRII flow cytometer. First division cells were observed as early as 36 hours after stimulation (and up to 96 hours after stimulation in some instances), so that the 72 hour time period shown in this figure demonstrates an example of first division daughter cell isolation. Time periods after activation can therefore vary between 36 hours and 96 hours. Of note, sorting first division daughter cells independent of the LIPSTIC label will enrich for distal daughter cells over an unstimulated product. This result was reproducible for CAR T cell targeting different antigens (e.g. CD19, gamma-delta TCR).

FIG. 4: Flow cytometry plot showing LIPSTIC labeled and unlabeled activated and non-activated CAR T cells. NTD=non-transduced (negative control), BBz=CD19bbz CAR, 28z=CD1928z CAR. This result was reproducible for CAR T cell targeting different antigens (e.g. CD19, gamma-delta TCR).

FIG. 5: Single cell RNA sequencing of proximal and distal daughter cells to establish differential transcriptional programs and functionality. 1) Gene expression library, 2) Surface proteome (Totalseq 205 markers, including 7 isotype controls), 3) LIPSTIC positivity (single molecule tracking). This result was reproducible for CAR T cell targeting different antigens (e.g. CD19, gamma-delta TCR).

FIG. 6: Post-library construction quantification of single cell libraries suggests differences in gene expression and surface proteome. Input 20,000 cells per group.

FIG. 7: The activation induced surface proteome is asymmetrically portioned after CAR T cell activation. Uniform manifold approximation and projection of surface proteome (based on surface protein antibodies).

FIG. 8: CD8 is inversely segregated in 1^{st} division CAR daughter cells compared to activation through the endogenous TCR. First column=protein name; second column=average LIPSTIC signal in LIPSTIC-negative (‘LIPSTIC−’) first division daughter cells, third column=adjusted P value comparing LIPSTIC− with LIPSTIC+ (LIPSTIC positive) first division daughter cells. This result was reproducible for CAR T cell targeting different antigens (e.g. CD19, gamma-delta TCR).

FIG. 9: Proximal daughter cells demonstrate higher levels of LIPSTIC, CD71, CD19 and CD10. First column=protein name; second column=adjusted P value comparing LIPSTIC+ with LIPSTIC− (LIPSTIC negative) first division daughter cells. This result was reproducible for CAR T cell targeting different antigens (e.g. CD19, gamma-delta TCR).

FIG. 10: Proximal 1^{st} division daughter cells demonstrate enriched expression of effector T cell genes, c-myc target genes, mTORC1 target genes, and genes involved in glycolysis. Gene set enrichment analysis demonstrating gene sets that demonstrate higher expression in proximal first division daughter cells. This result was reproducible for CAR T cell targeting different antigens (e.g. CD19, gamma-delta TCR).

FIG. 11: Distal 1^{st} division daughter cell gene expression resembles memory>naïve T cells. This result was reproducible for CAR T cell targeting different antigens (e.g. CD19, gamma-delta TCR).

FIG. 12: Activated CD8 CAR T cells establish global asymmetry of the cell surface proteome during the first cell division. Uniform manifold approximation and projection (UMAP) of 17215 single cells. Proximal and distal daughter cells, activated-undivided and resting T cells were sorted by flow cytometry 72 hours after activation (if applicable) as detailed in materials and methods. Cells were then stained with a custom DNA-barcoded antibody cocktail containing 205 antibodies. Cells were then processed according to the 10×5′V2 workflow and libraries for gene expression, surface antibody positivity and T cell receptor (VDJ) chains were sequenced on an Illumina Novaseq sequencer. Reads were aligned to a human reference and counted with the Cellranger pipeline. After quality control (using cut-offs for mitochondrial and ribosomal genes), dimensionality reduction was performed using the Seurat pipeline. Cell populations are separated by dotted lines. This result was reproducible for CAR T cell targeting different antigens (e.g. CD19, gamma-delta TCR).

FIGS. 13A-13B: Activated CD8 CAR T cells establish global asymmetry of the cell surface proteome during the first cell division. Uniform manifold approximation and projection (UMAP) of 17215 single cells displaying selected surface antibody signals. UMAP projection was performed as detailed for FIG. 12 and the surface positivity for selected antibodies for each cell is displayed. Names of respective surface antibodies are displayed at the top of each panel (FIG. 13A). FIG. 13B: Additionally, unsupervised clustering of the UMAP projection was performed demonstrating that cluster borders align with borders between resting, distal, proximal and activated-undivided cells. Dotted lines represent the border between proximal, distal, resting and activated-undivided cells. This result was reproducible for CAR T cell targeting different antigens (e.g. CD19, gamma-delta TCR).

FIG. 14: Asymmetric cell division in human CAR T cells exhibits unique features. Volcano plot of differential antibody positivity between proximal and distal first division daughter cells. Differentially expressed gene analysis was performed comparing the surface antibody positivity of clusters 2 and 9 from FIG. 13B (Seurat pipeline). Log 2-fold change of surface antibody positivity is displayed on the x-axis, adjusted log 10 p-value is shown on the y-axis. Dotted lines represent significance cut-offs for log 2-fold change and p-value. Selected antibody targets are displayed. Of note, surface CD8 is increased on distal daughter cells compared to proximal daughter cells, which differs from previous reports of T cells that had been stimulated through their endogenous T cell receptor. This result was reproducible for CAR T cell targeting different antigens (e.g. CD19, gamma-delta TCR).

FIG. 15: CAR T cells asymmetrically sort fate-associated transcripts. Uniform manifold approximation and projection (UMAP) of 17215 single cells displaying selected gene expression levels. UMAP projection was performed as detailed for FIGS. 12-13 and the expression level for selected genes for each cell is displayed in a color-coded format with light grey indicating low positivity and dark grey/black indicating high positivity. Dotted lines represent the border between proximal, distal, resting and activated-undivided cells. Name of respective gene displayed at the top of each panel. Selected genes represent canonical transcription factors or markers associated with naïve/memory, effector or tissue-resident T cells. Right panel: heat map comparing proximal and distal first division daughter cells. Each line represents one gene, each column represents one cell, colors indicated gene expression level as shown in the legend (log-fold change). The top 150 differentially expressed genes of proximal and distal daughter cells. This analysis demonstrates that the gene expression profile of proximal and distal daughter cells differs globally and that fate-associated transcripts are asymmetrically sorted during the first cell division after activation. This result was reproducible for CAR T cell targeting different antigens (e.g. CD19, gamma-delta TCR).

FIG. 16: Distal first division daughter cells demonstrate in vivo longevity and leukemia control. Left panel: experimental design; NSG mice were injected on day 0 with proximal or distal CD19 targeting CAR T daughter cells (2.5e5 cells per mouse) or control cells (same number of cells, nontransduced T cells from the same donor, unstimulated CAR T cells from the same donor). T cell numbers were evaluated on day 30 by flow cytometry and mice were injected on day 35 with 1e6 NALM6 leukemia cells to functionally challenge injected T cells and assess their longevity. All injections were performed intravenously. Right panel: Quantification of leukemia burden/rejection by in vivo bioluminescent quantification of NALM6 cells. NALM6 cells express click-beetle-green luciferase whose activity is used to quantify leukemia burden on indicated days. Y-axis displays bioluminescent signal in photons per second, x-axis displays time in days. Each line represents one mouse. Data representative of 2 separate experiments using T cells from 2 different healthy human donors. Right panel demonstrates longevity and superior leukemia control by distal first division daughter cells.

FIG. 17: Distal daughter cells demonstrate muted metabolic activity with preferential mitochondrial ATP production. Metabolic characterization of resting, distal and proximal daughter cells. The ATP production rate from oxidative phosphorylation (mitoATP) and glycolysis (glycoATP) was calculated from a Seahorse mitochondrial stress test (see material and methods). Mean and SD of 5 technical replicates per population is displayed. The percentage above each bar quantifies the proportion of ATP produced during glycolysis. This analysis demonstrates that distal daughter cells exhibit a muted metabolic activity (as shown by the overall reduced ATP production) and increased relative ATP production from oxidative phosphorylation (as shown by the lower glyco-ATP percentage) compared to proximal daughter cells, indicating metabolic asymmetry that is established during the first cell division after CAR T cell activation. This result was reproducible for CAR T cell targeting different antigens (e.g. CD19, gamma-delta TCR).

FIG. 18: Distal daughter cells pass through a transient state of increased cytotoxic potency. In vitro killing assays of proximal and distal daughter cells. Sorted proximal and distal daughter cells were co-incubated with target cells at increasing effector to target (E:T) ratios (displayed on the x-axis) for 4 hours. Nontransduced cells from the same donor served as negative control effector cells. Killing of target cells was assessed by bioluminescence imaging at the end of the assay with loss of luciferase activity correlating with relative target cell killing. Top panel: Killing assay performed on the day of the sort (day 0 after first cell division, i.e. day 3 after activation). Bottom panel: Killing assay performed on day 4 after the first cell division, i.e. day 7 after activation). Each dot represents the mean of a technical triplicate. This experiment demonstrates that proximal and distal daughter cells exhibit similar killing directly after cytogenesis. 4 days later, however, distal daughter cells demonstrate an increased decline in cytotoxicity compared to proximal daughter cells. This result was reproducible for CAR T cell targeting different antigens (e.g. CD19, gamma-delta TCR).

FIG. 19: Distal daughter cells demonstrate initial and long-term leukemia control at suboptimal CAR T cell dose. In vivo stress test of proximal and distal daughter first division CAR daughter cells. Right panel: experimental design; NSG mice were injected on day 0 with 1e6 NALM6 leukemia cells expressing click-beetle-green luciferase. On day 4, proximal or distal CD19 targeting CAR T daughter cells (2.5e5 cells per mouse) or control cells (nontransduced T cells from the same donor) were injected into NSG mice. Leukemia burden was repeatedly assessed by bioluminescence imaging. All injections were performed intravenously. Left panel: Quantification of leukemia burden/rejection by in vivo bioluminescent quantification of NALM6 cells. NALM6 cells express click-beetle-green luciferase whose activity is used to quantify leukemia burden on indicated days. Y-axis displays bioluminescent signal in photons per second, x-axis displays time in days. Each line represents one mouse. Data representative of 2 separate experiments using T cells from 2 different healthy human donors. Left panel demonstrates longevity and superior leukemia control by distal first division daughter cells.

### DETAILED DESCRIPTION

Early expansion and long-term persistence predict efficacy of genetically-engineered T cells. While this is thought to reflect successful induction of effector and memory T cell populations to provide both short-term clearance and long-lasting remission, the cellular mechanisms of fate induction after T cell activation through synthetic receptors are unknown. A better understanding of such processes could improve therapeutic outcome. Herein it is shown that human T cells engineered to express chimeric antigen receptors (CARs) undergo asymmetric cell division (ACD) with distinct proximal and distal daughter cells that adopt effector and memory phenotypes, respectively.

Using molecular proximity labeling to distinguish first division proximal and distal daughter cells, it was demonstrated that target-engaged CAR molecules remain on the proximal first division daughter cell and establish cellular asymmetry between daughter cells in size, proliferative pace, transcriptional program and metabolism. The single cell transcriptional program of proximal first division daughter cells is driven by c-myc, mTORC1, and JAK-STAT3 activation resulting in primarily glycolytic energy production, features consistent with effector T cell differentiation. Conversely, distal daughter cells utilize BACH-2, ETS-2, and KLF2 to shape their transcriptome, indicating a memory precursor phenotype that was accompanied by a predominance of oxidative phosphorylation metabolism.

Surprisingly, despite their memory precursor phenotype, first division distal daughter cells maintain similar cytolytic activity as proximal daughter cells for up to 48 hours after cytogenesis, uncovering a transient state of increased target sensitivity. This period of ‘target readiness’ is followed by a substantial decrease in cytotoxicity in distal, but not proximal daughter cells, highlighting functional plasticity as a hallmark of early memory CAR T cell differentiation. In vivo characterization of first division daughter cells in 2 separate xenograft leukemia mouse models confirms superior leukemia elimination and long-term persistence by distal daughter cells, functionally establishing these cells as memory precursors responsible for long-term efficacy of human CAR T cells. Collectively, these studies establish ACD as a novel framework for understanding mechanisms of CAR T cell differentiation and influencing therapeutic outcomes.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting. Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa and lambda light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present embodiments include, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

“Allogeneic” refers to any material derived from a different animal of the same species.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” as it is used in reference to a specific gene, refers to the gene that is naturally occurring in the organism, cell, tissue or system without the introduction of an exogenous or heterologous substance, such a nucleic acid molecule. For example, an “an endogenous TCR gene” refers to the gene encoding the TCR that is naturally occurring in the cell. “Endogenous” in reference to other materials, means that such material is from or produced inside an organism, cell, tissue or system without any exogenous material being introduced into the organism, cell, tissue, or system.

As used herein, the term “exogenous” refers to any material introduced from or produced outside an organism, cell, tissue or system. For example, a chimeric antigen receptor can be produced in a cell by the introduction of an exogenous nucleic acid molecule encoding the chimeric antigen receptor. A nucleic acid molecule that is introduced into the cell can also be referred to as a “heterologous” nucleic acid molecule.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence. The term “expression” as it is made in reference to a protein means the amounts of the protein that is present or made in a cell, organism, or system.

“Expression vector” refers to a vector comprising a recombinant polynucleotide comprising expression control sequences operatively linked to a nucleotide sequence to be expressed. An expression vector can comprise sufficient cis-acting elements for expression. In some embodiments, other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

When “an immunologically effective amount,” or “therapeutic amount” is indicated, the precise amount of the compositions to be administered can be determined by a physician or researcher with consideration of individual differences in age, weight, tumor size, extent of infection or metastasis, and condition of the patient (subject).

The term “immunosuppressive” is used herein to refer to reducing overall immune response.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Likewise, a cell is “isolated” when it's separated from coexisting materials of its natural state, or a particular cell or cell type can be “isolated” from a cell population when it is separated or removed from the population.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell. Molecules may be modified in many ways, including chemically, structurally, and functionally. Cells may be modified through the introduction of nucleic acids. In reference to a protein or gene, a modified protein or gene can refer to a protein or gene having a mutation, such as a insertion, deletion, point mutation, or any combination thereof.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

In the context of the present embodiments, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition or other composition provided for herein (e.g., viral vector), includes, e.g., subcutaneous (s.c.), intravenous (i.v.), intramuscular (i.m.), or intrasternal injection, or infusion techniques.

The term “polynucleotide” or “nucleic acid molecule” as used herein is defined as a chain of nucleotides. Furthermore, nucleic acid molecules are polymers of nucleotides. Thus, nucleic acid molecules and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a genomic nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny. For example, in some embodiments, the cell is transfected, transduced by a vector comprising the nucleic acid molecule. In some embodiments, the cell is transfected with a plasmid comprising the nucleic acid molecule.

To “treat” a disease as the term is used herein, means to reduce the frequency or severity of at least one sign or symptom of a disease or disorder experienced by a subject. As used in reference to a solid tumor, the term “treat” can be mean the reduction in the size of the solid tumor or in the number of locations a tumor is found either at its origin or that has metastasized.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects can be presented in a range format. It should be understood that the description in range format is merely for convenience and brevity and should not be construed as an inflexible limitation on the embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Methods

Provided herein are methods for distinguishing proximal and distal daughter CAR T cells. Also provided are methods of enriching distal daughter T cells (e.g., CAR T cells) or proximal daughter T cells (e.g., CAR T cells) from a population of T cells (e.g., population of CAR T cells). Also provided are methods of enriching, isolating and/or collecting distal daughter CAR T cells and use of the cells for treating a disease (e.g. cancer).

Distal daughter cells (e.g., T cells, e.g., CAR T cells) are “enriched” when the percentage of distal daughter cells in a population of cells (e.g., population of T cells, e.g., CAR T cells) is higher than the percentage of proximal daughter cells, or higher relative to the resting T cell population, or higher relative to a non-enriched daughter T cell population. For example, a population is “enriched for” distal daughter cells when over 50%, 60%, 70%, 80%, 90%, or 100% of the cells in the population are distal daughter cells (i.e. display the distal daughter cell phenotype). Likewise, proximal daughter cells (e.g., T cells, e.g., CAR T cells) are “enriched” when the percentage of proximal daughter cells in a population of cells (e.g., population of T cells, e.g., CAR T cells) is higher than the percentage of distal daughter cells. For example, a population is “enriched for” proximal daughter cells when over 50%, 60%, 70%, 80%, 90%, or 100% of the cells in the population are proximal daughter cells (i.e. display the proximal daughter cell phenotype).

In certain embodiments, methods for distinguishing or isolating or enriching distal daughter CAR T cells from proximal daughter CAR T cells are based on a modified version of the Labeling Immune Partnerships by SorTagging Intercellular Contacts (LIPSTIC) method. LIPSTIC, a proximity-based method for labeling proximal daughter cells (i.e. T cells) is described in detail in, for example, Pasqual et al. Nature volume 553, 496-500 (2018). Briefly, LIPSTIC is based on proximity-dependent labeling across cell-cell interfaces using the Staphylococcus aureus transpeptidase Sortase A (SrtA). SrtA covalently transfers a substrate containing the sorting motif LPXTG to a nearby N-terminal oligoglycine. In LIPSTIC, a ligand and receptor of interest are genetically fused to either SrtA or to a tag consisting of N-terminal glycine residues (e.g., G5). Addition of a SrtA substrate (e.g., an LPETG peptide linked at its N-terminus to a detectable label such as biotin or a fluorophore) leads to its loading onto SrtA on the donor cell via the formation of an acyl intermediate. Upon ligand-receptor interaction, SrtA catalyzes the transfer of the substrate onto the G5-tagged receptor. After cells separate, interaction history is revealed by the presence of the label on the surface of the G5-expressing cell.

In one aspect, the disclosure provides a method for distinguishing a distal first division daughter T cell (e.g., chimeric antigen receptor (CAR) T cell) from a proximal first division daughter T cell (e.g. CAR T cell). The method comprises i) pre-loading a LPETG peptide comprising a detectable label onto a Sortase A (SrtA) molecule, ii) fusing the SrtA to a target protein on a target cell, iii) tagging a T cell (e.g., CAR T cell) with an N-terminal glycine (e.g., penta-glycine (G5)) and labeling the T cell (e.g., CAR T cell) with a dye (alternatively, a CAR T cell that already expresses an amino-terminal glycine on the CAR can be labeled with the dye), iv) incubating the target cell with the T cell (e.g., CAR T cell), v) assessing T cell (e.g., CAR T cell) division by dye dilution or time, indicating daughter cell formation, vi) measuring the detectable label on the daughter cells following the first cell division of the T cell (e.g., CAR T cell), wherein when the detectable label is present, the cell is a proximal first division daughter cell, and wherein when detectable label is absent, the cell is a distal first division daughter cell. In certain embodiments, the T cell (e.g., CAR T cell) can be tagged with 1, 2, 3, 4, 5, 6, 7, 8, 9, or 10 glycines. In certain embodiments, the method can be used to enrich for distal daughter T cells (e.g., CAR T cells) from a population of CAR T cells. The distal daughter T cells (e.g., CAR T cells) can be isolated and/or collected. Distal daughter T cell (e.g., CAR T cells) isolated by the method can yield a purified population of distal daughter CAR T cells. The method can also generate an enriched population of distal daughter CAR T cells, i.e. wherein over 50%, 60%, 70%, 80%, 90%, or 100% of the cells in the population are distal daughter cells.

In certain embodiments of the method, the target protein is a tumor associated antigen (TAA), i.e. CD19, and the target cell is a cancer cell, i.e. a CD19+ cell. In certain embodiments, the target cell is any pathogenic target cell (e.g. comprising a target protein corresponding to an autoimmune, alloimmune, infectious, or fibrotic disease). In certain embodiments, the detectable label is a biotin or a fluorophore. In certain embodiments, the dye is selected from the group consisting of CellTrace™ Violet, CFSE, CellTrace™ Red, and CellTrace™ Yellow, or other labels such as fluorescently labeled histone proteins.

In certain embodiments, the method further comprises isolating and/or collecting the distal first division daughter cell. In certain embodiments, the method further comprises allowing the daughter cell to undergo a second division, and isolating and/or collecting the distal second division daughter cell. Cell division can be measured by further dye dilution using methods known in the art and/or disclosed herein. In certain embodiments, the method further comprises allowing the daughter cell to undergo a third division, and isolating and/or collecting the distal third division daughter cell. In certain embodiments, the method further comprises isolating and/or collecting the first, second, and/or third distal daughter cells.

While enrichment or isolation of distal and proximal first division daughter cells is feasible as described above, one can similarly enrich or isolate the entire first cell division population independent of the LIPSTIC label, or the second or third cell division populations can be enriched or isolated. Similarly, one can replace dye dilution with appropriate time frames as a surrogate for cell divisions since e.g. 96 hours after stimulation 2-3 cell divisions occur.

Accordingly, another aspect of the disclosure provides a method of enriching or isolating a distal daughter CAR T cell, or population thereof. The method comprises stimulating a CAR T cell with a target cell, and isolating the CAR T cell progeny for up to 7 days after stimulation. The key points of this method are: 1) the CAR does not need to have a glycine tag, 2) the target cell does not need to have a sortase-tagged antigen, 3) dye dilution is not required, and 4) target cells (live or irradiated), or the target antigen conjugated to beads, are incubated with CAR T cells. In certain embodiments, CAR T cell progeny are collected 1 day, 2 days, 3 days, 4 days, 5 days, 6 days, or 7 days after stimulation. Collected is a population of both proximal and distal daughter cells that is relatively enriched for distal daughter cells (i.e. wherein over 50%, 60%, 70%, 80%, 90%, or 100% of the cells in the population are distal daughter cells) compared to an unstimulated population or a population that is allowed to expand for longer periods of time.

Previous studies have shown that asymmetric 1¹ cell division determines functional differentiation, longevity, and metabolism after T cell activation in mice. Typically, proximal daughter T cells have the following characteristics/markers: CD8^{high}, develops into effector cell, CD62L^{low}, PKCζ^{low}, rapid proliferation, larger, CD25^{high}, Myc^{high}, CD98^{high}, glycolytic metabolism, and short-lived in vivo. Distal daughter T cells typically have the following characteristics/markers: CD8^{low}, develops into memory cell, CD62L^{high}, PKCζ^{high}, slow proliferation, smaller, CD25^{low}, Myc^{low}, CD98^{low}, aerobic metabolism, and long-lived in vivo (Science 23 Mar. 2007:Vol. 315, Issue 5819; Immunity. 2011; 34:492-504; Nature. 2016 Apr. 21; 532(7599): 389-93; Science Immunology 2019, Vol 4, Issue 34). However, this study reports the surprising finding that in CAR T cells, CD8 and the endogenous T cell receptor levels are elevated in the distal daughter cells, not the proximal daughter cells, which is the opposite to what is found in natural T cells. Therefore, one aspect of the present disclosure provides a method for identifying or detecting a distal daughter CAR T cell (or distinguishing it from a proximal daughter CAR T cell), by measuring the level of CD8 on the cell, wherein when the CD8 level is high or elevated compared to a control (i.e. a proximal daughter cell), the cell is a distal daughter CAR T cell.

In another aspect, the disclosure provides a method for distinguishing proximal from distal daughter T cells (e.g., chimeric antigen receptor (CAR) T cells) in a population of cells e.g., population of CAR T cells). The method comprises measuring a set of genes expressed by the CAR T cell(s). When at least 1, 2, 3, 4, 5, 10, 15, 20, or 30 of the genes expressed on the cell surface are selected from the following list, then the cells are proximal daughter cells: CD30, CD71, CD25, CD106, CD117 (c-kit), CD88 (C5aR), CDllc, CD155 (PVR), CD223 (LAG-3), CD146, CD163.1, CD19.1, CD194 (CCR4), CD23, Notch1, CD105, CD169 (Sialoadhesin Siglec-1), CD83.1, aCD207, CD137 (4-1BB), TCRdeltagamma, CD134 (OX40), B7-H4, CD324E (Cadherin), CD122 (IL-2R beta), CD10, CD206 (MMR), CD178 (Fas-L), CD82.1, CD141 (Thrombomodulin), CD80.1, LOX-1, CD1a, IgGFc, CD123, TCRValpha24, CD209 (DC-SIGN), CD272 (BTLA), CD304 (Neuropilin-1), CD85 (jILT2), CD252 (OX40L), CD303 (BDCA2), IgM, TSLPRTSLP-R, CD98, CD34.1, CD20, CD235ab, CD62L, CD144VE (Cadherin), CD307d (FcRL4), CD197 (CCR7), CD201 (EPCR), CD54, CX3CR1.1, CD360 (IL-21R), CD140b (PDGFRbeta), CD112 (Nectin-2), CD124 (IL-4Ralpha), CD257 (BAFFBLYS), CD335 (NKp46), CD152 (CTLA-4), EGFR1, GARPLRRC32, CD62E, CD269 (BCMA), CD158 (KIR2DL1S1S3S5), Podoplanin, XCR1.1, CD70.1, CD254 (TRANCERANKL), Podocalyxin, CD158f (KIR2DL5), CD274 (B7-H1 PD-L1), CD66b, CD21, CD307 (eFcRL5), CD268 (BAFF-R), CD154, CD137L (4-1BB Ligand), CD119 (IFN gamma R alpha chain), CD1 d, CD370 (CLEC9ADNGR1), CD267 (TACI), CD107a (LAMP-1), CD24.1, CD13, TCRVgamma9, CD357 (GITR), TCRVdelta2, Notch3, CD40.1, CD326 (Ep-CAM), CD204, Fc epsilon RI alpha, CD294 (CRTH2), CD158el (KIR3DL1, NKB1), CD150 (SLAM), CD14.1, Ig light chain kappa, LIPSTIC1, CD184 (CXCR4), CD196 (CCR6), CD79b (IgB), CD16, DR3TRAMP, CD319 (CRACC), CD258 (LIGHT), CD32, TCRVbeta131, CD275 (B7-H2, ICOSL), CD45R/B220, CD279, CD35, CD42b, CD366 (Tim-3), CD336 (NKp44), CD140a (PDGFRalpha), IgD, CD62P (P-Selectin), Mac-2, CD1c, CD41, CD11b, CD185 (CXCR5), CD22.1, CD328 (Siglec-7), CD325 (N-Cadherin), CD86.1, CD36.1, CD158b (KIR2DL2/L3, NKAT2, KLRG1, MAFA), Ig light chain lambda, GPR56, TIGIT/VSTM3, CD337 (NKp30), CD64, CD33.1, TCRValpha72, CD270 (HVEMTR2), CD4.1, HLAA2, CD183 (CXCR3), CD58 (LFA-3).

When at least 1, 2, 3, 4, 5, 10, 15, 20, or 30 of the genes expressed on the cell surface are selected from the following list, then the cells are distal daughter cells: CD103 (Integrin alpha E), CD45RA, CD99.1, TCRalpha/beta, CD101 (BB27), CD8, CD49a, CD7.1, CD48.1, CD52.1, CD73, CD5.1, CD3, CD224, CD45, CD47.1, CD11a, CD18, CD31, CD27.1, mouse CD49f, CD2.1, CD26, CD195 (CCR5), CD38.1, CD244 (2B4), CD29, CD314 (NKG2D), CD305 (LAIR1), CD352 (NTB-A), CD49d, CD95Fas, CD69.1, integrin beta 7, CD44.1, CD273B7 (DCPD-L2), CD45RO, CD96 (TACTILE), CD57 Recombinant, CD94, CD56, CD49b, CD226 (DNAM-1), CD278 (ICOS), CD127 (IL-7R), CLEC12A.1, CD39, CD161, HLA-DR, CD81 (TAPA-1), HLA-E.1, HLA-ABC, CD66ace, CD28.1.

In another aspect, the disclosure provides a method of identifying or enriching for a distal daughter CAR T cell or population thereof (e.g. from a population of CAR T cells). The method comprises measuring a set of genes expressed in a CAR T cell or population thereof. The genes are selected from the group consisting of CD103 (Integrin alpha E), CD45RA, CD99.1, TCRalpha/beta, CD101 (BB27), CD8, CD49a, CD7.1, CD48.1, CD52.1, CD73, CD5.1, CD3, CD224, CD45, CD47.1, CD11a, CD18, CD31, CD27.1, mouse CD49f, CD2.1, CD26, CD195 (CCR5), CD38.1, CD244 (2B4), CD29, CD314 (NKG2D), CD305 (LAIR1), CD352 (NTB-A), CD49d, CD95Fas, CD69.1, integrin beta 7, CD44.1, CD273B7 (DCPD-L2), CD45RO, CD96 (TACTILE), CD57 Recombinant, CD94, CD56, CD49b, CD226 (DNAM-1), CD278 (ICOS), CD127 (IL-7R), CLEC12A.1, CD39, CD161, HLA-DR, CD81 (TAPA-1), HLA-E.1, HLA-ABC, CD66ace, CD28.1. When at least one of these genes are expressed, then the cell is identified as a distal daughter CAR T cell. The method can further comprise isolating and/or collecting the distal daughter cell or population thereof.

In certain embodiments, CAR T cells are stimulated on target cells or antigen (no G5 tag or sortase or dilution dye is required), and distal daughter cells are selected by positively sorting on a distal daughter cell marker such as IL7R.

In certain embodiments, the disclosure provides a method for enriching a distal daughter CAR T cell relative to a proximal daughter CAR T cell. The method comprises positively sorting on a distal daughter cell marker such as IL7R, stimulating the CAR T cells on target cells, followed by collection of daughter cells within certain timeframe (i.e., 1-7 days) in absence of the LPETG/sortase method.

Another aspect of the disclosure provides a method for inducing CAR T cells to adopt a defined daughter cell fate (either proximal or distal) by transiently overexpressing a transcription factor or panel of transcription factors in primary human chimeric antigen receptor (CAR) T cells or unmodified T cells. This transient overexpression influences the first cell division after target cell encounter, causing both daughter cells to adopt the same cell fate (either proximal or distal). The described methods involve the delivery of transcription factors using electroporation of protein, mRNA or circular RNA, or as mRNA or circular RNA encapsulated in lipid nanoparticles (LNPs). Transcription factors can be delivered individually or as mixtures with defined ratios. This improves the efficacy and longevity of CAR T cell therapies and immunotherapies for treating cancer and other diseases.

CAR T cell therapy is a groundbreaking treatment for cancer and autoimmune patients. However, one of the limitations of this therapy is the variable persistence and functionality of CAR T cells after infusion into patients. The first cell division after target cell encounter is known to be asymmetric, with the proximal daughter cell developing into a short-lived effector cell and the distal daughter cell into a long-lived memory cell. Enhancing the longevity of both daughter cells could potentially improve the efficacy and durability of CAR T cell therapy.

In certain embodiments, the disclosure provides methods for transiently overexpressing transcription factors, including STAT1, STAT2, KDM5B, MXD4, MAX, KLF2, FLT1, IRF2, IRF7, IRF3, TRF9, ELK1, ELK4, ZNF358, and IKZF1, in primary human CAR T cells or unmodified T cells. The overabundance of these transcription factors prior to and during the first cell division influences the first cell division after target cell encounter, leading to both daughter cells adopting the properties of the distal daughter cell (i.e., becoming long-lived). Conversely, temporary over-expression of transcription factors associated with proximal daughter cell fate (MYC, TP73, MYBL1, SP2, YBX1, E2F2, E2F7, E2F8, NFYB) forces both daughter cells to adopt a proximal daughter cell phenotype and behavior.

Any method known in the art can be used for delivering the transcription factors. In certain embodiments, the transcription factor is delivered via electroporation of protein, mRNA or circular RNA. In another embodiment, the transcription factor is delivered as mRNA or circular RNA encapsulated in lipid nanoparticles (LNPs). In certain embodiments, the transcription factors are delivered individually or as mixtures.

The disclosure provides methods for transiently overexpressing a panel of transcription factors in primary human CAR T cells or unmodified T cells. These transcription factors include, but are not limited to, STAT1, STAT2, KDM5B, MXD4, MAX, KLF2, FLI1, IRF2, IRF7, IRF3, IRF9, ELK1, ELK4, ZNF358, and IKZF1 to enforce distal daughter cell fate. Conversely, delivery and transient expression of MYC, TP73, MYBL1, SP2, YBX1, E2F2, E2F7, E2F, NFYB will enforce a proximal daughter cell fate. Surface molecule transient overexpression:

In addition to the transient overexpression of transcription factors, surface molecules (as outlined above) can also be transiently overexpressed using similar methods prior to T cell infusion. This approach further enhances the properties of the CAR T cells or unmodified T cells.

Alternatively, the transcription factors can be pharmacologically modified (by treating T cells with agonists or antagonists) prior to the infusion of CAR T cells. This strategy modulates the activity of the transcription factors during the first cell division and subsequently impacts the CAR T cell properties and behavior.

The temporary overexpression or disruption of transcription factor RNA or DNA can also be achieved by transient expression of dCas9 fused to a co-activator or repressor domain or transient expression of Cas13 protein (achieved by delivery of mRNA or protein in ribonucleoprotein complexes) in conjunction with guide RNAs targeting the transcription factors above (resulting in mRNA destruction). These gene-editing techniques provide alternative methods for transiently modulating the expression of the desired transcription factors in CAR T cells or unmodified T cells during a sensitive period of fate assumption.

In conclusion, provided herein are a variety of methods for transiently overexpressing or suppressing transcription factors or surface molecules, pharmacologically modifying transcription factors, or utilizing dCas9 or Cas13 for transient overexpression or disruption of transcription factors or surface proteins in primary human CAR T cells or unmodified T cells. These strategies will enhance the longevity and functionality of CAR T cells or unmodified T cells, improving the efficacy and durability of CAR T cell therapies and immunotherapies for treating cancer and other diseases.

Methods for enriching cells, e.g., T cells, e.g. CAR T cells, are well known in the art. In some embodiments, one or more of the T cell populations is enriched for or depleted of cells that are positive for (marker+) or express high levels (marker^{high}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD8+, CD45RA+, and/or CD45RO+ T cells, are isolated by positive or negative selection techniques. In some cases, such markers are those that are absent or expressed at relatively low levels on certain populations of T cells (such as proximal daughter cells) but are present or expressed at relatively higher levels on certain other populations of T cells (such as distal daughter cells). In one embodiment, the cells are enriched for cells that are positive or expressing high surface levels of CD45RA, CD45RO, CCR7, CD28, CD27, CD44, CD127, and/or CD62L and/or depleted of cells that are positive for or express high surface levels of CD45RA. In some embodiments, cells are enriched for or depleted of cells positive or expressing high surface levels of CD122, CD95, CD25, CD27, and/or IL7-Ra (CD127). In some embodiments, CD8+ T cells are enriched for cells positive for CD45RO (or negative for CD45RA) or enriched for CD45RA (negative for CD45RO) and for CD62L. In some embodiments, T cells may be enriched for expression of both CD45RA and CD45RO. For example, CD3+, CD28+ T cells can be positively selected using CD3/CD28 conjugated magnetic beads (e.g., DYNABEADS® M-450 CD3/CD28 T Cell Expander). The enrichment of specific cell populations such as the distal or proximal first division daughter cells can be accomplished using various techniques, one of which is magnetic enrichment or magnetic-activated cell sorting (MACS). The process involves the use of magnetic particles that are bound to antibodies specific for certain cell surface markers. In the context of T cell subsets, markers like CD45RA, CD45RO, CD127, and CD62L are often used to differentiate between naïve, memory, and effector T cells.

CD45RA and CD45RO are isoforms of the same protein and are often used to distinguish between naïve (CD45RA+) and memory (CD45RO+) T cells. CD127 is the alpha chain of the IL-7 receptor and is expressed at high levels on naïve and memory T cells but is downregulated on effector T cells. CD62L (L-selectin) is a homing receptor that enables T cells to enter secondary lymphoid organs; it's highly expressed on naïve T cells and central memory T cells, but downregulated in effector T cells.

In the MACS process, cells are first incubated with a specific antibody conjugated to small magnetic particles. The antibody targets the cell surface marker of interest (e.g., CD45RA, CD45RO, CD127, or CD62L). The cell suspension is then placed in a magnetic field, and the magnetically labeled cells are retained while the unlabeled cells are washed away. The retained cells are then eluted once the magnetic field is removed, resulting in a highly enriched population of cells expressing the targeted marker.

The distinction between positive and negative sorting is a significant aspect of magnetic-activated cell sorting (MACS). In positive selection, the magnetic particles are attached to an antibody that recognizes the marker of interest. The cells expressing this specific marker are retained in the magnetic field and constitute the positively selected population. Conversely, negative selection involves the use of antibodies against markers not expressed by the cells of interest. These antibodies are bound to the magnetic particles, and when the cell suspension is placed in a magnetic field, the cells expressing these markers are retained, while the cells not expressing these markers (i.e., the cells of interest) are collected in the flow-through. This method is often used when the goal is to obtain a population of cells that are not activated or altered by antibody binding.

For example, to isolate naive T cells using negative selection, a cocktail of antibodies against markers that are not expressed by naive T cells (such as CD45RO) can be used, so that the naive T cells are collected in the flow-through.

### C. Modified Immune Cells

The present disclosure provides modified immune cells or precursors thereof (e.g., T cells) for use in immunotherapy (e.g. CAR T cells). In certain aspects, the disclosure provides a distal daughter CAR T cell, or population thereof, isolated by any of the methods disclosed herein.

The modified cell can comprise any CAR known in the art as well as those described in detail elsewhere herein. A CAR comprises affinity for an antigen on a target cell. Accordingly, such modified cells possess the specificity directed by the CAR that is expressed therein.

In some embodiments, the cells or populations of cells comprise distal first division daughter cells, distal second division daughter cells, and/or distal third division daughter cells.

In some embodiments, the immune cells in the composition retain a phenotype of the immune cell or cells compared to the phenotype of cells in a corresponding or reference composition when assessed under the same conditions. In some embodiments, cells in the composition include effector memory cells, central memory cells, stem central memory cells, effector memory cells, and long-lived effector memory cells. In some embodiments, such property, activity or phenotype can be measured in an in vitro assay, such as by incubation of the cells in the presence of an antigen targeted by the CAR, a cell expressing the antigen and/or an antigen-receptor activating substance. In some embodiments, any of the assessed activities, properties or phenotypes can be assessed at various days following electroporation or other introduction of the agent, such as after or up to 3, 4, 5, 6, 7 days. In some embodiments, such activity, property or phenotype is retained by at least 80%, 85%, 90%, 95% or 100% of the cells in the composition compared to the activity of a corresponding composition containing cells engineered with the recombinant receptor but not comprising the genetic disruption of the targeted gene when assessed under the same conditions.

As used herein, reference to a “corresponding composition” or a “corresponding population of immune cells” (also called a “reference composition” or a “reference population of cells”) refers to immune cells (e.g., T cells) obtained, isolated, generated, produced and/or incubated under the same or substantially the same conditions, except that the immune cells or population of immune cells were not introduced with the agent. In some aspects, except for not containing introduction of the agent, such immune cells are treated identically or substantially identically as immune cells that have been introduced with the agent, such that any one or more conditions that can influence the activity or properties of the cell, including the upregulation or expression of the inhibitory molecule, is not varied or not substantially varied between the cells other than the introduction of the agent.

Methods and techniques for assessing the expression and/or levels of T cell markers are known in the art. Antibodies and reagents for detection of such markers are well known in the art, and readily available. Assays and methods for detecting such markers include, but are not limited to, flow cytometry, including intracellular flow cytometry, ELISA, ELISPOT, cytometric bead array or other multiplex methods, Western Blot and other immunoaffinity-based methods. In some embodiments, CAR-expressing cells can be detected by flow cytometry or other immunoaffinity based method for expression of a marker unique to such cells, and then such cells can be co-stained for another T cell surface marker or markers.

In some embodiments, the cells, compositions and methods provide for the deletion, knockout, disruption, or reduction in expression of the target gene in immune cells (e.g. T cells) to be adoptively transferred (such as cells engineered to express a CAR). In some embodiments, the methods are performed ex vivo on primary cells, such as primary immune cells (e.g. T cells) from a subject. In some aspects, methods of producing or generating such genetically engineered T cells include introducing into a population of cells containing immune cells (e.g. T cells) one or more nucleic acid encoding a CAR and an agent or agents that is capable of disrupting, a gene that encode the endogenous receptor to be targeted. As used herein, the term “introducing” encompasses a variety of methods of introducing DNA into a cell, either in vitro or in vivo, such methods including transformation, transduction, transfection (e.g. electroporation), and infection. Vectors are useful for introducing DNA encoding molecules into cells. Possible vectors include plasmid vectors and viral vectors. Viral vectors include retroviral vectors, lentiviral vectors, or other vectors such as adenoviral vectors or adeno-associated vectors.

The population of cells containing T cells can be cells that have been obtained from a subject, such as obtained from a peripheral blood mononuclear cells (PBMC) sample, an unfractionated T cell sample, a lymphocyte sample, a white blood cell sample, an apheresis product, or a leukapheresis product. In some embodiments, T cells can be separated or selected to enrich T cells in the population using positive or negative selection and enrichment methods. In some embodiments, the population contains CD4+, CD8+ or CD4+ and CD8+ T cells. In some embodiments, subsequent to introduction of the CAR, the cells are cultured or incubated under conditions to stimulate expansion and/or proliferation of cells.

Thus, provided are cells, compositions and methods that enhance an immune cell, such as T cell, function in adoptive cell therapy, including those offering improved efficacy, such as by increasing activity and potency of administered genetically engineered cells, while maintaining persistence or exposure to the transferred cells over time. In some embodiments, the genetically engineered cells, exhibit increased expansion and/or persistence when administered in vivo to a subject, as compared to certain available methods. In some embodiments, the provided immune cells exhibit increased persistence when administered in vivo to a subject. In some embodiments, the persistence of genetically engineered immune cells, in the subject upon administration is greater as compared to that which would be achieved by alternative methods, such as those involving administration of cells genetically engineered by methods in which T cells were not introduced with an agent that reduces expression of or disrupts a gene encoding an endogenous receptor. In some embodiments, the persistence is increased at least or about at least 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In certain embodiments, the immune cell or precursor cell thereof is a T cell. In certain embodiments, the T cell is a human T cell. In certain embodiments, the cell is an autologous cell (e.g. an autologous T cell). In certain embodiments, the cell is an allogenic cell (e.g. an allogenic T cell).

The modified cells can comprise any chimeric antigen receptor (CAR) known in the art or disclosed herein.

Thus, provided are cells, compositions and methods that enhance immune cell, such as T cell, function in adoptive cell therapy, including those offering improved efficacy, such as by increasing activity and potency of administered genetically engineered cells, while maintaining persistence or exposure to the transferred cells over time.

In some embodiments, the degree or extent of persistence of administered cells can be detected or quantified after administration to a subject. For example, in some aspects, quantitative PCR (qPCR) is used to assess the quantity of cells expressing the CAR in the blood or serum or organ or tissue (e.g., disease site) of the subject. In some aspects, persistence is quantified as copies of DNA or plasmid encoding the exogenous receptor per microgram of DNA, or as the number of receptor-expressing cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the modified cell can be used to distinguish the administered cells from endogenous cells in a subject.

The cells provided for herein can also be generated in vivo by administering a vector, such as a virus to transduce the T cell in vivo. The T cell can be targeted by modifying the vector with a targeting moiety. Examples of transduction systems and vectors that can be used to produce the cells in vivo are described in, but not limited to, U.S. Publication Application No. 20210353543, U.S. Pat. Nos. 10,064,958, 9,486,539, PCT Publication No. WO/2021/202604, U.S. Publication No. 20210228627, U.S. Publication No. 20210198698, and U.S. Publication No. 20210283179, each of which is hereby incorporated by reference in its entirety.

### D. Methods of Treatment

The modified cells described herein (e.g., distal daughter CAR T cells) may be included in a composition for immunotherapy for treating solid tumors. The composition may include a pharmaceutical composition and further include a pharmaceutically acceptable carrier. A therapeutically effective amount of the pharmaceutical composition comprising the modified cells may be administered.

In some embodiments, the disclosure includes a method of treating a solid tumor in a subject in need thereof, comprising administering to the subject a population of modified immune cells or precursor cells thereof (e.g. distal daughter CAR T cells) disclosed herein. In another aspect, the embodiments are provided that include a method for adoptive cell transfer therapy comprising administering to a subject in need thereof a modified immune cell or precursor cell thereof as provided herein (e.g. distal daughter CAR T cells).

Methods for administration of immune cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The modified immune cells can be administered to an animal, preferably a mammal, even more preferably a human, to treat a cancer. In addition, the cells of can be used for the treatment of any condition related to a cancer, especially a cell-mediated immune response against a tumor cell(s), where it is desirable to treat or alleviate the disease. The types of cancers to be treated with the modified cells or pharmaceutical compositions provided herein include, but are not limited to, carcinoma, blastoma, and sarcoma, and certain leukemia or lymphoid malignancies, benign and malignant tumors, and malignancies e.g., sarcomas, carcinomas, and melanomas. Other exemplary cancers include but are not limited breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. Adult tumors/cancers and pediatric tumors/cancers are also included. In one embodiment, the cancer is a carcinoma. In one embodiment, the cancer is a sarcoma. In one embodiment, the cancer is a leukemia. In one embodiment the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

In certain exemplary embodiments, the modified immune cells provided for herein are used to treat a melanoma, or a condition related to melanoma. Examples of melanoma or conditions related thereto include, without limitation, superficial spreading melanoma, nodular melanoma, lentigo maligna melanoma, acral lentiginous melanoma, amelanotic melanoma, or melanoma of the skin (e.g., cutaneous, eye, vulva, vagina, rectum melanoma). In one embodiment, a method of the present disclosure is used to treat cutaneous melanoma. In one embodiment, a method of the present disclosure is used to treat refractory melanoma. In one embodiment, a method of the present disclosure is used to treat relapsed melanoma.

In yet other exemplary embodiments, the modified immune cells provided for herein are used to treat a sarcoma, or a condition related to sarcoma. Examples of sarcoma or conditions related thereto include, without limitation, angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, and synovial sarcoma. In one embodiment, a method of the present disclosure is used to treat synovial sarcoma. In one embodiment, a method of the present disclosure is used to treat liposarcoma such as myxoid/round cell liposarcoma, differentiated/dedifferentiated liposarcoma, and pleomorphic liposarcoma. In one embodiment, a method of the present disclosure is used to treat myxoid/round cell liposarcoma. In one embodiment, a method of the present disclosure is used to treat a refractory sarcoma. In one embodiment, a method of the present disclosure is used to treat a relapsed sarcoma.

The cells to be administered may be autologous, with respect to the subject undergoing therapy. In some embodiments, the cells are allogeneic with respect to the subject undergoing therapy.

The administration of the cells may be carried out in any convenient manner known to those of skill in the art. The cells may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells are injected directly into a site of inflammation in the subject, a local disease site in the subject, a lymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁻ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between about 1×10⁴ and about 1×10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁻ and/or CD8⁺ cells/kg body weight, for example, at or about 1×10⁵ CD4⁻ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4+ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In certain embodiments, the modified cells (e.g., a modified cell comprising a CAR) may be administered to a subject in combination with an immune checkpoint antibody (e.g., an anti-PD1, anti-CTLA-4, or anti-PDL1 antibody). For example, the modified cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In certain embodiments, the modified cell may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the modified cell comprising the CAR. In certain embodiments, combination treatment comprising an immune checkpoint modulator may increase the therapeutic efficacy of a therapy comprising a modified cell.

Following administration of the cells, the biological activity of the engineered cell populations in some embodiments is measured, e.g., by any of a number of known methods.

Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to CAR T cell therapy. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine. In some embodiments, a specific dosage regimen of the present disclosure does not include a lymphodepletion step prior to the administration of the modified T cells.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

Dosing of lymphodepletion chemotherapy may be scheduled on Days −6 to −4 (with a −1 day window, i.e., dosing on Days −7 to −5) relative to T cell (e.g., CAR-T, TCR-T, a modified T cell, etc.) infusion on Day 0.

In an exemplary embodiment, for a subject having cancer, the subject receives lymphodepleting chemotherapy including 300 mg/m² of cyclophosphamide by intravenous infusion 3 days prior to administration of the modified T cells. In an exemplary embodiment, for a subject having cancer, the subject receives lymphodepleting chemotherapy including 300 mg/m² of cyclophosphamide by intravenous infusion for 3 days prior to administration of the modified T cells.

In an exemplary embodiment, for a subject having cancer, the subject receives lymphodepleting chemotherapy including fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including fludarabine at a dose of 30 mg/m² for 3 days.

In an exemplary embodiment, for a subject having cancer, the subject receives lymphodepleting chemotherapy including cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including cyclophosphamide at a dose of about 300 mg/m²/day, and fludarabine at a dose of 30 mg/m² for 3 days.

The modified cells can be administered in dosages and routes and at times to be determined in appropriate pre-clinical and clinical experimentation and trials. Cell compositions may be administered multiple times at dosages within these ranges. Administration of the cells may be combined with other methods useful to treat the desired disease or condition as determined by those of skill in the art.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the embodiments provide for, following the diagnosis of CRS, appropriate CRS management strategies to mitigate the physiological symptoms of uncontrolled inflammation without dampening the antitumor efficacy of the engineered cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

As such, the modified immune cells provided for herein when used in a method of treatment as described herein, enhances the ability of the modified immune cells in carrying out their function. Accordingly, the embodiments provided for herein provide a method for enhancing a function of a modified immune cell for use in a method of treatment as described herein.

In one aspect, the embodiments include a method of treating cancer in a subject in need thereof, comprising administering to the subject any one of the modified immune or precursor cells provided for herein. Yet another aspect of the embodiments include a method of treating cancer in a subject in need thereof, comprising administering to the subject a modified immune or precursor cell generated by any one of the methods disclosed herein.

In the various embodiments of the methods disclosed herein, the subject can be administered any CAR known in the art or disclosed herein. The CAR can be specific for any tumor associated antigen (TAA) or tumor specific antigen (TSA) known to one of ordinary skill in the art.

### E. Chimeric Antigen Receptors

The present disclosure provides compositions and methods for modified immune cells or precursors thereof, e.g., modified T cells, comprising a chimeric antigen receptor (CAR). CARs disclosed herein comprise an antigen binding domain, a transmembrane domain, and an intracellular domain. In certain embodiments, the CAR T cell comprises a glycine modification (e.g. an n-terminal glycine tag). In certain embodiments, the CAR T cell is tagged with 1, 2, 3, 4, 5, 6, 7, 8, 9, or 10 glycines. In certain embodiments, the CAR T cell comprises a penta-glycine (G5) modification (e.g. an n-terminal penta-glycine (G5) tag). In certain embodiments, the CAR T cell comprises a dye. In certain embodiments, the CAR T cell is a distal daughter cell isolated by any of the methods disclosed herein.

The antigen binding domain of the CAR may be operably linked to another domain of the CAR, such as the transmembrane domain or the intracellular domain for expression in the cell. In some embodiments, a first nucleic acid sequence encoding the antigen binding domain is operably linked to a second nucleic acid encoding a transmembrane domain, and further operably linked to a third a nucleic acid sequence encoding an intracellular domain.

The antigen binding domains described herein can be combined with any of the transmembrane domains described herein or known, any of the intracellular domains or cytoplasmic domains described herein or known, or any of the other domains described herein that may be included in a CAR. A CAR may also include a hinge domain. A CAR may also include a spacer domain. In some embodiments, each of the antigen binding domain, transmembrane domain, and intracellular domain is separated by a linker.

### Antigen Binding Domain

The antigen binding domain of a CAR is an extracellular region of the CAR for binding to a specific target antigen including proteins, carbohydrates, and glycolipids. In some embodiments, the CAR comprises affinity to a target antigen on a target cell. The target antigen may include any type of protein, or epitope thereof, associated with the target cell. For example, the CAR may comprise affinity to a target antigen on a target cell that indicates a particular disease state of the target cell.

In some embodiments, the target cell antigen is a tumor associated antigen (TAA). Examples of tumor associated antigens (TAAs), include but are not limited to, CD19, differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3CA 27.29BCAA, CA 195, CA 242, CA-50, CAM43, CD68P1, CO-029, FGF-5, G250, Ga733EpCAM, HTgp-175, M344, MA-50, MG7-Ag, MOV18, NB/70K, NY-CO-1, RCAS1, SDCCAG16, TA-90Mac-2 binding proteincyclophilin C-associated protein, TAAL6, TAG72, TLP, and TPS. In some embodiments, the antigen binding domain of the CAR targets an antigen that includes but is not limited to CD19, CD20, CD22, ROR1, Mesothelin, CD33/IL3Ra, c-Met, PSMA, PSCA, Glycolipid F77, EGFRvIII, GD-2, MY-ESO-1 TCR, MAGE A3 TCR, and the like.

Depending on the desired antigen to be targeted, the CAR can be engineered to include the appropriate antigen binding domain that is specific to the desired antigen target. For example, if CD19 is the desired antigen that is to be targeted, an antibody for CD19 can be used as the antigen bind moiety for incorporation into the CAR.

In some embodiments, the target cell antigen is CD19. As such, in some embodiments, a CAR has affinity for CD19 on a target cell. This should not be construed as limiting in any way, as a CAR having affinity for any target antigen is suitable for use in a composition or method.

As described herein, a CAR of the present disclosure having affinity for a specific target antigen on a target cell may comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin. In some embodiments, a CAR having affinity for CD19 on a target cell may comprise a CD19 binding domain.

In some embodiments, a CAR may have affinity for one or more target antigens on one or more target cells. In some embodiments, a CAR may have affinity for one or more target antigens on a target cell. In such embodiments, the CAR is a bispecific CAR, or a multispecific CAR. In some embodiments, the CAR comprises one or more target-specific binding domains that confer affinity for one or more target antigens. In some embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through an oligo- or polypeptide linker, an Fc hinge region, or a membrane hinge region.

In some embodiments, the antigen binding domain is selected from the group consisting of an antibody, an antigen binding fragment (Fab), and a single-chain variable fragment (scFv). In some embodiments, a CD19 binding domain is selected from the group consisting of a CD19-specific antibody, a CD19-specific Fab, and a CD19-specific scFv. In one embodiment, a CD19 binding domain is a CD19-specific antibody. In one embodiment, a CD19 binding domain is a CD19-specific Fab. In one embodiment, a CD19 binding domain is a CD19-specific scFv.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. In some embodiments, the antigen binding domain portion comprises a mammalian antibody or a fragment thereof. The choice of antigen binding domain may depend upon the type and number of antigens that are present on the surface of a target cell.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen binding domain (e.g., PSCA binding domain) comprises an scFv having the configuration from N-terminus to C-terminus, VH-linker-VL. In some embodiments, the antigen binding domain comprises an scFv having the configuration from N-terminus to C-terminus, VL-linker-VH. Those of skill in the art would be able to select the appropriate configuration. In certain embodiments, the scFv comprises SEQ ID NO: 20. In certain embodiments, the scFv comprises SEQ ID NO: 23.

The linker can be rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO: 24), (GGGS)ₙ (SEQ ID NO: 25), and (GGGGS)ₙ (SEQ ID NO: 26), where n represents an integer of at least 1. Exemplary linker sequences can comprise amino acid sequences including, without limitation, GGSG (SEQ ID NO:27), GGSGG (SEQ ID NO:28), GSGSG (SEQ ID NO:29), GSGGG (SEQ ID NO:30), GGGSG (SEQ ID NO:31), GSSSG (SEQ ID NO:32), GGGGS (SEQ ID NO:33), GGGGSGGGGSGGGGS (SEQ ID NO:34) and the like. Those of skill in the art would be able to select the appropriate linker sequence. In some embodiments, an antigen binding domain comprises a heavy chain variable region (VH) and a light chain variable region (VL), wherein the VH and VL are separated by a linker sequence having the amino acid sequence GGGGSGGGGSGGGGS (SEQ ID NO:34), which may be encoded by the nucleic acid sequence GGTGGCGGTGGCTCGGGCGGTGGTGGGTCGGGTGGCGGCGGATCT (SEQ ID NO: 14).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In some embodiments, the antigen binding domain may be derived from the same species in which the CAR will ultimately be used. For example, for use in humans, the antigen binding domain of the CAR may comprise a human antibody or a fragment thereof.

In some embodiments, the antigen binding domain may be derived from a different species in which the CAR will ultimately be used. For example, for use in humans, the antigen binding domain of the CAR may comprise a murine antibody or a fragment thereof.

In certain embodiments, the antigen binding domain comprises a heavy chain variable region that comprises three heavy chain complementarity determining regions (HCDRs) and a light chain variable region that comprises three light chain complementarity determining regions (LCDRs).

In certain embodiments, the CAR comprises an antigen binding domain capable of binding CD19, wherein the antigen binding domain is a scFv comprises the amino acid sequence set forth in SEQ ID NO: 20.

Tolerable variations of the antigen binding domain sequences will be known to those of skill in the art. For example, in some embodiments the antigen binding domain comprises an amino acid sequence that has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 20 or 23.

### Transmembrane Domain

CARs may comprise a transmembrane domain that connects the antigen binding domain of the CAR to the intracellular domain of the CAR. The transmembrane domain of a CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In some embodiments, the transmembrane domain is naturally associated with one or more of the domains in the CAR. In some embodiments, the transmembrane domain can be selected or modified by one or more amino acid substitutions to avoid binding of such domains to the transmembrane domains of the same or different surface membrane proteins, to minimize interactions with other members of the receptor complex.

The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane domain include, without limitation, transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen binding domains described herein, any of the intracellular domains described herein, or any of the other domains described herein that may be included in a subject CAR.

In some embodiments, the transmembrane domain further comprises a hinge region. A CAR of may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, a CAR includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell (Hudecek et al., supra). The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 an to about 10 aa, from about 10 an to about 15 aa, from about 15 an to about 20 aa, from about 20 an to about 25 aa, from about 25 an to about 30 aa, from about 30 an to about 40 aa, or from about 40 an to about 50 aa. In some embodiments, the hinge region can have a length of greater than 5 aa, greater than 10 aa, greater than 15 aa, greater than 20 aa, greater than 25 aa, greater than 30 aa, greater than 35 aa, greater than 40 aa, greater than 45 aa, greater than 50 aa, greater than 55 aa, or more.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge regions can have a length of greater than 20 amino acids (e.g., 30, 40, 50, 60 or more amino acids).

For example, hinge regions include glycine polymers (G)., glycine-serine polymers (including, for example, (GS)ₙ, (GSGGS)ₙ (SEQ ID NO: 24) and (GGGS)ₙ (SEQ ID NO:25), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). Exemplary hinge regions can comprise amino acid sequences including, but not limited to, GGSG (SEQ ID NO:27), GGSGG (SEQ ID NO:28), GSGSG (SEQ ID NO:29), GSGGG (SEQ ID NO:30), GGGSG (SEQ ID NO:31), GSSSG (SEQ ID NO:32), and the like.

In some embodiments, the hinge region is an immunoglobulin heavy chain hinge region. Immunoglobulin hinge region amino acid sequences are known in the art; see, e.g., Tan et al., Proc. Nal. Acad. Sci. USA (1990) 87(1):162-166; and Huck et al., Nucleic Acids Res. (1986) 14(4): 1779-1789. As non-limiting examples, an immunoglobulin hinge region can include one of the following amino acid sequences: DKTHT (SEQ ID NO:35); CPPC (SEQ ID NO:36); CPEPKSCDTPPPCPR (SEQ ID NO:37) (see, e.g., Glaser et al., J. Biol. Chem. (2005) 280:41494-41503); ELKTPLGDTTHT (SEQ ID NO:38); KSCDKTHTCP (SEQ ID NO:39); KCCVDCP (SEQ ID NO:40); KYGPPCP (SEQ ID NO:41); EPKSCDKTHTCPPCP (SEQ ID NO:42) (human IgG1 hinge); ERKCCVECPPCP (SEQ ID NO:43) (human IgG2 hinge); ELKTPLGDTTHTCPRCP (SEQ ID NO:44) (human IgG3 hinge); SPNMVPHAHHAQ (SEQ ID NO:45) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO:46); see, e.g., Yan et al., J. Biol. Chem. (2012) 287: 5891-5897. In one embodiment, the hinge region can comprise an amino acid sequence derived from human CD8, or a variant thereof.

### Intracellular Signaling Domain

A CAR also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

Examples of an intracellular domain include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcyRIII, FcsRI, cytoplasmic tails of Fc receptors, an immunoreceptor tyrosine-based activation motif (ITAM) bearing cytoplasmic receptors, and combinations thereof.

In some embodiments, the intracellular signaling domain of the CAR includes any portion of one or more co-stimulatory molecules, such as at least one signaling domain from CD2, CD3, CD8, CD27, CD28, ICOS, 4-1BB, PD-1, any derivative or variant thereof, any synthetic sequence thereof that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11 d, ITGAE, CD103, ITGAL, CD11a, LFA-1, ITGAM, CDlib, ITGAX, CD11c, ITGBl, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a CAR include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a CAR include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs.

In some embodiments, intracellular signaling domains includes the signaling domains of human immunoglobulin receptors that contain immunoreceptor tyrosine based activation motifs (ITAMs) such as, but not limited to, FcgammaRI, FcgammaRIIA, FcgammaRIIC, FcgammaRIIIA, FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceRl gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCIZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in a CAR of the present disclosure includes a DAP10/CD28 type signaling chain. In one embodiment, an intracellular signaling domain suitable for use in a CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the antigen binding domains described herein, any of the transmembrane domains described herein, or any of the other domains described herein that may be included in the CAR.

In some embodiments, the CAR comprises the amino acid sequence set forth in SEQ ID NO: 3 or 4. In some embodiments, the CAR is encoded by a nucleic acid sequence selected set forth in SEQ ID NO: 7 or 8.

Tolerable variations of the CAR sequences will be known to those of skill in the art. For example, in some embodiments the CAR comprises an amino acid sequence that has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NO: 3 or 4. In some embodiments the CAR is encoded by a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least or 99% sequence identity to the nucleic acid sequence set forth in SEQ ID NO: 7 or 8.

The embodiments can include any one of: a CAR, a nucleic acid encoding a CAR, a vector comprising a nucleic acid encoding a CAR, a cell comprising a CAR, a cell comprising a nucleic acid encoding a CAR, and a cell comprising a vector comprising a nucleic acid encoding a CAR.

### F. Methods of Producing Genetically Modified Immune Cells

The present disclosure provides methods for producing or generating a modified immune cell or precursor thereof (e.g., a CAR T cell), e.g., for adoptive immunotherapy. In certain embodiments, the cells generally are engineered by introducing into the cell one or more nucleic acids encoding the CAR.

In certain embodiments, the immune cell or precursor cell thereof is a T cell. In certain embodiments, the T cell is human T cell. In certain embodiments, T cell is an autologous T cell.

In some embodiments, a nucleic acid molecule encoding the CAR is introduced into a cell by an expression vector. Expression vectors comprising a nucleic acid sequence encoding a CAR of the are also provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggybak, and Integrases such as Phi31. Some other suitable expression vectors include Herpes simplex virus (HSV) and retrovirus expression vectors.

In certain embodiments, the nucleic acid encoding a CAR is introduced into the cell via viral transduction. In some embodiments, the viral transduction is performed in vivo. Examples of in vivo transduction to introduce a heterologous nucleic acid molecule can be found, for example, in U.S. Application No., which is hereby incorporated by reference in its entirety. The in vivo transduction can be used to introduce the nucleic acid molecule encoding the CAR.

In certain embodiments, the viral transduction comprises contacting the immune or precursor cell with a viral vector comprising the nucleic acid encoding an exogenous CAR. In certain embodiments, the viral vector is an adeno-associated viral (AAV) vector. In certain embodiments, the AAV vector comprises a 5′ ITR and a 3′ITR derived from AAV6. In certain embodiments, the AAV vector comprises a Woodchuck Hepatitis Virus post-transcriptional regulatory element (WPRE). In certain embodiments, the AAV vector comprises a polyadenylation (polyA) sequence. In certain embodiments, the polyA sequence is a bovine growth hormone (BGH) polyA sequence.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the CAR in the host cell. In some embodiments, the adenovirus genome is a 36 kb, linear, double stranded DNA, where a foreign DNA sequence (e.g., a nucleic acid encoding an exogenous CAR) may be inserted to substitute large pieces of adenoviral DNA in order to make the expression vector (see, e.g., Danthinne and Imperiale, Gene Therapy (2000) 7(20): 1707-1714).

Another expression vector is based on an adeno associated virus (AAV), which takes advantage of the adenovirus coupled systems. This AAV expression vector has a high frequency of integration into the host genome. It can infect nondividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retroviral vector is constructed by inserting a nucleic acid (e.g., a nucleic acid encoding a CAR) into the viral genome at certain locations to produce a virus that is replication defective. Though the retroviral vectors are able to infect a broad variety of cell types, integration and stable expression of the CAR requires the division of host cells.

Lentiviral vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the Human Immunodeficiency Viruses (HIV-1, HIV-2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are capable of infecting non-dividing cells and can be used for both in vivo and ex vivo gene transfer and expression, e.g., of a nucleic acid encoding a CAR (see, e.g., U.S. Pat. No. 5,994,136).

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell an immune cell or precursor thereof, e.g., a T cell, an NK cell, or an NKT cell.

Embodiments provided for herein also provide genetically engineered cells (e.g. cells with a mutated or disrupted CD5 gene), which include and stably express a CAR of the present disclosure. In some embodiments, the genetically engineered cells are genetically engineered T-lymphocytes (T cells), naive T cells (TN), memory T cells (for example, central memory T cells (TCM), effector memory cells (TEM)), natural killer cells (NK cells), and macrophages capable of giving rise to therapeutically relevant progeny. In certain embodiments, the genetically engineered cells are autologous cells.

Modified cells (e.g., comprising (expressing) a CAR may be produced by stably transfecting host cells with an expression vector including a nucleic acid of the present disclosure. These can also be produced in vivo by administering a viral particle that can infect such cells in vivo to produce the modified cells in vivo. Examples of in vivo transduction to introduce a heterologous nucleic acid molecule can be found, for example, in U.S.

Application No., which is hereby incorporated by reference in its entirety. Additional methods for generating a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a CAR of the present disclosure may be expanded ex vivo or expanded in vivo by administering other therapeutics that can stimulate the expansion of the modified cell.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York. Chemical methods for introducing an expression vector into a host cell include colloidal dispersion systems, such as macromolecule complexes, nanocapsules, microspheres, beads, and lipid-based systems including oil-in-water emulsions, micelles, mixed micelles, and liposomes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform may be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). Compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

In some embodiments, the nucleic acids introduced into the host cell are RNA. In some embodiments, the RNA is mRNA that comprises in vitro transcribed RNA or synthetic RNA. The RNA may be produced by in vitro transcription using a polymerase chain reaction (PCR)-generated template. DNA of interest from any source can be directly converted by PCR into a template for in vitro mRNA synthesis using appropriate primers and RNA polymerase. The source of the DNA may be, for example, genomic DNA, plasmid DNA, phage DNA, cDNA, synthetic DNA sequence or any other appropriate source of DNA.

PCR may be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers may also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

In some embodiments, the RNA is electroporated into the cells, such as in vitro transcribed RNA. Any solutes suitable for cell electroporation, which can contain factors facilitating cellular permeability and viability such as sugars, peptides, lipids, proteins, antioxidants, and surfactants can be included.

In some embodiments, a nucleic acid encoding a CAR of the present disclosure will be RNA, e.g., in vitro synthesized RNA. Methods for in vitro synthesis of RNA are known in the art; any known method can be used to synthesize RNA comprising a sequence encoding a CAR. Methods for introducing RNA into a host cell are known in the art. See, e.g., Zhao et al. Cancer Res. (2010) 15: 9053. Introducing RNA comprising a nucleotide sequence encoding a CAR into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a CAR.

The disclosed methods can be applied to the modulation of T cell activity in basic research and therapy, in the fields of cancer, stem cells, acute and chronic infections, and autoimmune diseases, including the assessment of the ability of the genetically modified T cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

RNA transfection methods can be used without a vector, such as a plasmid or a virus. An RNA transgene, such as those encoding for the CAR can be delivered to a lymphocyte and expressed therein following a cell activation, as a minimal expressing cassette without the need for any additional viral sequences. Cloning of cells may not be necessary because of the efficiency of transfection of the RNA and its ability to uniformly modify the entire lymphocyte population.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

In some embodiments, the immune cells (e.g. T cells) can be incubated or cultivated prior to, during and/or subsequent to introducing the nucleic acid molecule encoding the exogenous receptor (e.g., CAR). In some embodiments, the cells (e.g. T cells) can be incubated or cultivated prior to, during or subsequent to the introduction of the nucleic acid molecule encoding the exogenous receptor, such as prior to, during or subsequent to the transduction of the cells with a viral vector (e.g. lentiviral vector) encoding the exogenous receptor.

### G. Sources of Immune Cells

In some embodiments, a source of immune cells is obtained from a subject (e.g. for ex vivo manipulation). Sources of cells manipulation may also include, e.g., autologous or allogeneic donor blood, cord blood, or bone marrow. For example the source of immune cells may be from the subject to be treated with the modified immune cells, e.g., the subject's blood, the subject's cord blood, or the subject's bone marrow. Non-limiting examples of subjects include humans, dogs, cats, mice, rats, and transgenic species thereof. Preferably, the subject is a human.

The cells may also be created by transducing the cells in vivo, such as, but not limited to, by the methods described herein. In some embodiments, the viral transduction can be directed to certain immune cells by incorporating a targeting moiety into the viral particle.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, if performed ex vivo, the methods include isolating immune cells from the subject, preparing, processing, culturing, and/or engineering/modifying them. In some embodiments, preparation of the engineered cells includes one or more culture and/or preparation steps. The cells for engineering/modifying as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for cells that are positive or expressing high surface levels of CD45RA, CD45RO, CCR7, CD28, CD27, CD44, CD 127, and/or CD62L and/or depleted of cells that are positive for or express high surface levels of CD45RA. In some embodiments, cells are enriched for or depleted of cells positive or expressing high surface levels of CD 122, CD95, CD25, CD27, and/or IL7-Ra (CD 127). In some embodiments, CD8+ T cells are enriched for cells positive for CD45RO (or negative for CD45RA) or enriched for CD45RA (negative for CD45RO) and for CD62L. In some embodiments, T cells may be enriched for expression of both CD45RA and CD45RO. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD 14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CDllb, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering/modification. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL. In certain embodiments, the modified cells are expanded without any stimulating agents. In certain embodiments, the modified cells are expanded in vivo.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁻ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### H. Expansion of T Cells

In certain embodiments, after initial activation the CAR T cells provided for herein can be multiplied by about 2 fold, 4 fold, 8 fold, 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In some embodiments embodiment, the modified T cells expand in the range of about 20 fold to about 50 fold. In some embodiments, the modified T cells divide exactly once, twice, three times and/or four time prior to isolation or are stimulated for equivalent time periods to reach the defined number of cell divisions.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In some embodiments, the cells are cryopreserved or the expanded cells are cryopreserved. The cryopreserved T cells are thawed prior to introducing nucleic acids into the T cell.

In another embodiment, the method comprises isolating T cells and expanding the T cells. In another embodiment, the methods further comprises cryopreserving the T cells prior to expansion. In yet another embodiment, the cryopreserved T cells are thawed for electroporation with the RNA encoding the chimeric membrane protein. These introductions can be done before or after the cell is modified to mutate or otherwise disrupt the CD5 gene.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the T cells may include an agent that can co-stimulate the T cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more by culturing the electroporated population.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

### I. Pharmaceutical Compositions and Formulations

Also provided herein are populations of immune cells (e.g. distal daughter CAR T cells) and compositions containing such cells and/or enriched for such cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for intravenous, intraperitoneal, subcutaneous, pulmonary, transdermal, intramuscular, intranasal, buccal, sublingual, or suppository administration. In some embodiments, the pharmaceutical compositions are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells or viral particles in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present application describes various embodiments, it should be understood by those skilled in the art that various changes may be made and equivalents may be substituted without departing from the true spirit and scope of the embodiments. It will be readily apparent to those skilled in the art that other suitable modifications and adaptations of the compositions and methods described herein may be made using suitable equivalents without departing from the scope of the embodiments disclosed herein. In addition, many modifications may be made to adapt a particular situation, material, composition of matter, process, process step or steps, to the objective, spirit and scope of the embodiments provided for herein. All such modifications are intended to be within the scope of the embodiments provided for herein and claims appended hereto. Having now described certain embodiments in detail, the same will be more clearly understood by reference to the following examples, which are included for purposes of illustration only and are not intended to be limiting.

### EXPERIMENTAL EXAMPLES

The embodiments are now described with reference to the following Examples. These Examples are provided for the purpose of illustration only, and the embodiments are not limited to these Examples, but rather encompasses all variations that are evident as a result of the teachings provided herein.

Sequences used in the experiments include, but are not limited to:

CD19 sortase amino acid sequence:
(SEQ ID NO: 1)
Signal peptide
(SEQ ID NO: 9)
Flag tag
(SEQ ID NO: 10)

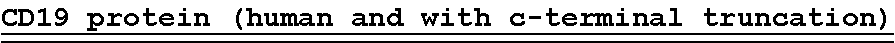

(SEQ ID NO: 11)

[symbol]

(SEQ ID NO: 12)

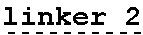

(SEQ ID NO: 13)
cloning site
sortase mutant
(SEQ ID NO: 15)

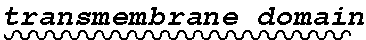

(SEQ ID NO: 16)
MEFGLSWLFLVAILKGVQCGS [symbol] EQAKPQIPKDKSKVAGYIEIPD
ADIKEPVYPGPATSEQLNRGVSFAEENESLDDQNISIAGHTFIDRPNYQFTNLKAAKK
GSMVYFKVGNETRKYKMTSIRNVKPTDVGVLDEQKGKDKQLTLITCDDYNEKTGVW

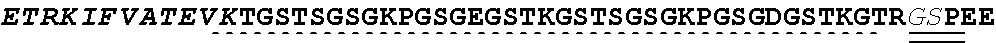

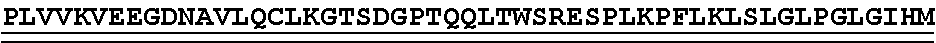

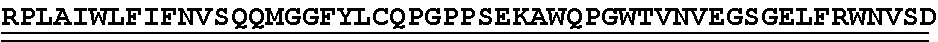

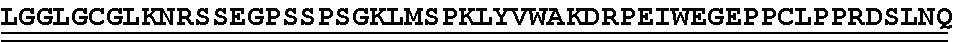

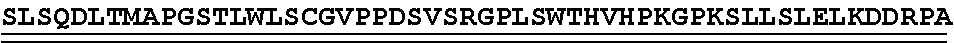

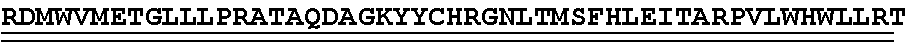

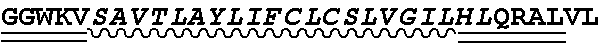

γδTCR sortase (clone: PEER) amino acid sequence:
(SEQ ID NO: 2)
Signal peptide
(SEQ ID NO: 9)
Flag tag
(SEQ ID NO: 10)

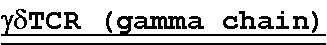

(SEQ ID NO: 17)

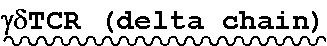

(SEQ ID NO: 18)

[symbol]

(SEQ ID NO: 12)

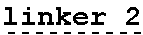

(SEQ ID NO: 13)

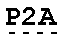

(SEQ ID NO: 19)
cloning site
sortase mutant
(SEQ ID NO: 15)
MEFGLSWLFLVAILKGVQCGS [symbol] EQAKPQIPKDKSKVAGYIEIPD
ADIKEPVYPGPATSEQLNRGVSFAEENESLDDQNISIAGHTFIDRPNYQFTNLKAAKK
GSMVYFKVGNETRKYKMTSIRNVKPTDVGVLDEQKGKDKQLTLITCDDYNEKTGVW

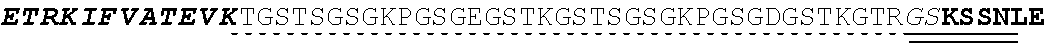

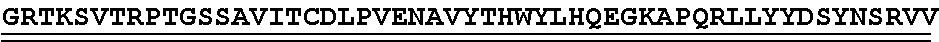

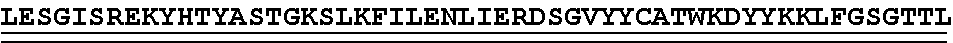

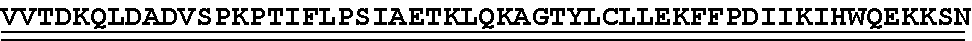

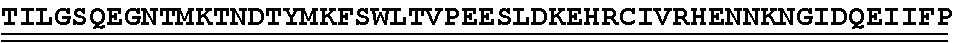

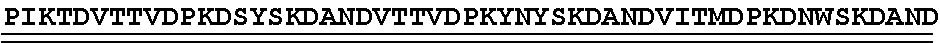

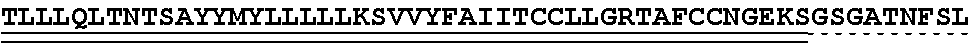

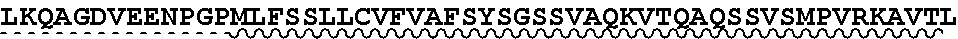

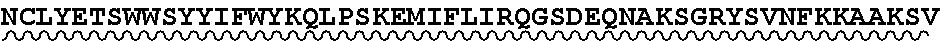

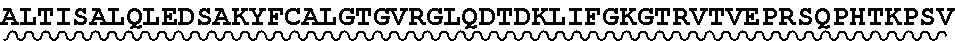

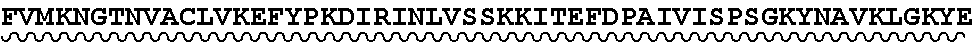

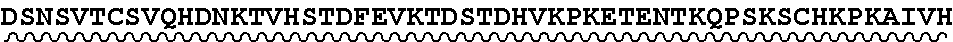

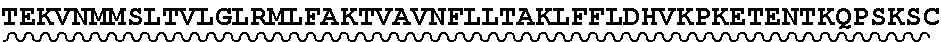

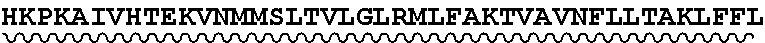

HPG5-bbz CAR (anti-γδ) amino acid sequence:
(SEQ ID NO: 3)
scFV
(SEQ ID NO: 20)
CAR hinge, transmembrane, cytoplasmic domain
(SEQ ID NO: 21)
cloning site
Signal peptide
(SEQ ID NO: 9)

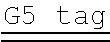

(SEQ ID NO: 22)

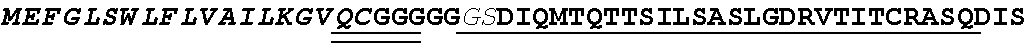

NYLNWYQQNPDGTVKLLIYYTSRLHSGVPSRESGSGSGTEYSLTIKNLEQEDIAT
YFCQQGNMVPFTFGSGTKLEIKGGGGSGGGGSGGGGSQVOLKESGPGLVAPS
QSLSITCTVSGFSLTSYGVHWVRQPPGKGLEWLGVIWASGTTDYNSALMSRLTI
SKDNSKSQVFLRMNSLOTDDTAMYYCARETTASFGYWGLGTLVTVSAASTTTP
APRPPTPAPTIASQPLSLRPEACRPAAGGAVHTRGLDFACDSGIYIWAPLAGTCGVLL
LSLVITLYCKRGRKKLLYIFKQPFMRPVQTTQEEDGCSCRFPEEEEGGCELRVKFSRS
ADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
CD19G5-bbz CAR amino acid sequence:
(SEQ ID NO: 4)
scFV
(SEQ ID NO: 23)
CAR hinge, transmembrane, cytoplasmic domain
(SEQ ID NO: 21)
cloning site
Signal peptide
(SEQ ID NO: 9)

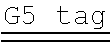

(SEQ ID NO: 22)

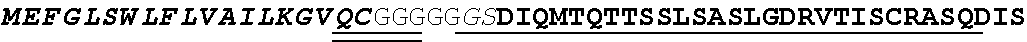

KYLNWYQQKPDGTVKLLIYHTSRLHSGVPSRFSGSGSGTDYSLTISNLEQEDIAT
YFCQQGNTLPYTFGGGTKLEITGGGGSGGGGSGGGGSEVKLQESGPGLVAPSQ
SLSVTCTVSGVSLPDYGVSWIROPPRKGLEWLGVIWGSETTYYNSALKSRLTIIK
DNSKSQVFLKMNSLOTDDTAIYYCAKHYYYGGSYAMDYWGQGTSVTVSSASTT
TPAPRPPTPAPTIASQPLSLRPEACRPAAGGAVHTRGLDFACDSGIYIWAPLAGTCGV
LLLSLVITLYCKRGRKKLLYIFKQPFMRPVQTTQEEDGCSCRFPEEEEGGCELRVKFS
RSADAPAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPR
gdTCR sortase DNA sequence (clone: PEER):
(SEQ ID NO: 5)
ATGGAGTTTGGGCTGAGCTGGCTTTTTCTTGTGGCTATTTTAAAAGGTGTCCAGT
GCGGATCTGACTATAAAGATGATGATGATAAAGGGAGCGGCCTGGAGCAAGCCA
AACCCCAAATTCCCAAGGATAAGAGTAAGGTTGCAGGTTATATAGAGATACCCG
ATGCAGACATAAAGGAGCCGGTATATCCTGGGCCCGCCACGAGCGAACAGCTCA
ACAGGGGAGTATCATTTGCAGAAGAGAACGAGTCCCTTGATGACCAAAACATAA
GTATCGCAGGACATACCTTCATCGACCGCCCGAATTATCAGTTCACTAACCTCAA
AGCCGCGAAAAAGGGGTCCATGGTATATTTCAAAGTAGGAAATGAGACTAGAAA
ATATAAAATGACTTCTATTCGAAATGTCAAGCCCACGGACGTTGGTGTACTCGAT
GAGCAAAAAGGCAAAGACAAACAACTCACGCTCATCACTTGCGACGATTACAAT
GAAAAGACAGGCGTCTGGGAAACACGCAAAATCTTCGTGGCAACAGAGGTCAA
GACGGGTAGCACCAGTGGTAGTGGCAAACCTGGTTCTGGTGAAGGAAGCACCAA
AGGGAGCACTAGCGGCAGCGGAAAGCCAGGCAGTGGGGATGGAAGTACGAAAG
GAACTCGCGGATCCAAGTCAAGTAATTTGGAGGGTAGGACAAAGAGTGTAACCA
GGCCAACCGGAAGTTCTGCCGTGATTACATGTGACCTTCCGGTAGAAAATGCTGT
TTATACACATTGGTATCTGCACCAAGAGGGTAAAGCACCGCAAAGGCTCTTGTA
CTATGACAGTTACAACTCTAGGGTAGTTTTGGAGAGTGGGATTAGTAGGGAGAA
ATATCACACATACGCATCTACGGGCAAATCTCTGAAATTTATTCTCGAGAATCTT
ATAGAGCGAGACTCAGGGGTTTATTATTGCGCCACCTGGAAAGACTATTATAAG
AAATTGTTTGGATCTGGCACGACGCTTGTAGTGACGGATAAGCAACTTGATGCCG
ATGTTTCACCAAAACCCACCATTTTCTTGCCATCTATCGCGGAGACCAAACTGCA
GAAGGCAGGTACATATCTTTGTCTCTTGGAAAAATTCTTTCCGGACATAATAAAG
ATCCACTGGCAGGAGAAGAAGAGCAACACTATCCTTGGGTCACAAGAAGGCAAT
ACGATGAAAACGAATGATACATATATGAAGTTTTCTTGGCTGACCGTTCCAGAG
GAAAGCTTGGATAAGGAACACAGGTGCATAGTAAGACATGAGAATAACAAGAA
CGGGATCGACCAAGAAATCATATTCCCACCGATTAAGACTGATGTGACTACGGT
GGACCCCAAGGACTCCTACTCCAAAGATGCGAACGACGTAACTACGGTAGACCC
CAAATATAACTACAGTAAAGACGCAAATGACGTGATAACCATGGACCCAAAGGA
CAACTGGAGTAAGGATGCTAACGATACGCTGCTGCTTCAGCTTACGAATACTAGT
GCTTATTACATGTACCTGCTGCTTCTGCTCAAGAGTGTAGTCTATTTTGCCATCAT
TACGTGTTGCCTTCTTGGACGCACGGCCTTTTGCTGCAACGGTGAGAAATCCGGG
TCAGGTGCTACTAACTTCAGTCTTCTGAAACAAGCAGGAGATGTGGAAGAAAAC
CCTGGTCCTATGTTGTTTTCAAGCCTGCTCTGCGTATTCGTGGCTTTTTCCTACTCT
GGTTCTTCCGTCGCTCAGAAGGTCACCCAAGCGCAGAGTAGCGTATCCATGCCTG
TTCGGAAAGCGGTGACACTCAACTGTCTCTACGAAACGAGCTGGTGGTCCTATTA
TATCTTCTGGTACAAGCAACTCCCTAGCAAGGAAATGATATTCTTGATACGCCAA
GGCTCCGATGAGCAGAATGCAAAATCCGGTCGCTATAGCGTAAATTTTAAGAAG
GCCGCGAAAAGCGTTGCCCTCACCATCAGTGCATTGCAGCTCGAAGACAGCGCA
AAATACTTCTGTGCACTCGGAACTGGGGTGAGAGGTTTGCAGGACACTGATAAA
CTTATATTTGGGAAAGGCACTCGAGTCACTGTCGAGCCCCGCTCACAGCCACAC
ACCAAGCCCTCCGTATTTGTTATGAAAAACGGCACCAATGTAGCATGCCTCGTGA
AAGAGTTCTATCCCAAAGACATAAGGATCAATCTCGTGTCTTCAAAAAAAATCA
CAGAATTCGATCCCGCAATCGTCATAAGCCCGTCAGGTAAGTACAATGCTGTGA
AACTGGGAAAGTACGAGGATTCCAACTCTGTGACTTGTTCAGTGCAGCATGATA
ACAAGACCGTTCATTCAACCGACTTTGAAGTCAAAACAGATAGTACAGACCACG
TAAAGCCAAAAGAGACTGAAAATACAAAGCAACCTTCTAAGTCATGCCACAAGC
CCAAGGCTATAGTGCATACAGAGAAGGTCAACATGATGAGCTTGACGGTTCTCG
GCCTCCGAATGCTTTTTGCCAAGACTGTCGCCGTAAACTTTCTCCTCACAGCAAA
ACTGTTTTTTCTCTGA
CD19 sortase DNA sequence:
(SEQ ID NO: 6)
ATGGAGTTTGGGCTGAGCTGGCTTTTTCTTGTGGCTATTTTAAAAGGTGTCCAGT
GCGGATCTGACTATAAAGATGATGATGATAAAGGGAGCGGCCTGGAGCAAGCCA
AACCCCAAATTCCCAAGGATAAGAGTAAGGTTGCAGGTTATATAGAGATACCCG
ATGCAGACATAAAGGAGCCGGTATATCCTGGGCCCGCCACGAGCGAACAGCTCA
ACAGGGGAGTATCATTTGCAGAAGAGAACGAGTCCCTTGATGACCAAAACATAA
GTATCGCAGGACATACCTTCATCGACCGCCCGAATTATCAGTTCACTAACCTCAA
AGCCGCGAAAAAGGGGTCCATGGTATATTTCAAAGTAGGAAATGAGACTAGAAA
ATATAAAATGACTTCTATTCGAAATGTCAAGCCCACGGACGTTGGTGTACTCGAT
GAGCAAAAAGGCAAAGACAAACAACTCACGCTCATCACTTGCGACGATTACAAT
GAAAAGACAGGCGTCTGGGAAACACGCAAAATCTTCGTGGCAACAGAGGTCAA
GACGGGTAGCACCAGTGGTAGTGGCAAACCTGGTTCTGGTGAAGGAAGCACCAA
AGGGAGCACTAGCGGCAGCGGAAAGCCAGGCAGTGGGGATGGAAGTACGAAAG
GAACTCGCGGATCCCCCGAGGAACCCCTCGTAGTTAAGGTAGAGGAGGGTGACA
ACGCTGTCCTTCAATGCCTGAAAGGCACATCTGACGGCCCTACACAGCAATTGA
CCTGGTCTCGCGAATCACCGCTTAAGCCGTTCCTGAAACTCTCTCTCGGGCTCCC
AGGACTTGGCATCCACATGCGCCCCCTTGCCATTTGGCTTTTCATATTCAATGTAT
CTCAGCAAATGGGCGGCTTCTATCTGTGTCAGCCGGGGCCGCCCTCCGAAAAAG
CCTGGCAGCCGGGCTGGACCGTTAATGTTGAGGGCTCAGGCGAGTTGTTCCGCTG
GAACGTAAGTGATTTGGGTGGTCTGGGTTGTGGGCTGAAAAACAGAAGCTCCGA
AGGGCCCTCCAGCCCTTCTGGGAAACTGATGAGCCCTAAGTTGTACGTCTGGGC
GAAGGATCGGCCCGAGATTTGGGAGGGCGAGCCACCATGTCTGCCCCCGCGGGA
TTCACTGAACCAGTCCCTTTCACAAGACCTCACAATGGCGCCCGGCTCCACGCTC
TGGTTGAGTTGTGGTGTGCCTCCTGACTCCGTCAGCAGGGGACCCCTTTCATGGA
CACATGTTCACCCTAAAGGACCGAAAAGCCTCCTCTCACTCGAGCTTAAGGATG
ATCGACCTGCCAGAGACATGTGGGTCATGGAGACCGGGCTGCTCCTTCCGAGAG
CGACGGCTCAGGACGCGGGGAAGTATTATTGTCACCGCGGAAATCTCACCATGA
GTTTCCATTTGGAAATAACCGCTCGCCCAGTCCTCTGGCATTGGCTGCTTCGGAC
TGGAGGGTGGAAAGTAAGCGCCGTTACCCTGGCATACCTCATCTTTTGCTTGTGC
TCCCTGGTCGGCATACTGCACCTTCAACGCGCGCTCGTTCTCTGATAA
HPG5bbz DNA sequence:
(SEQ ID NO: 7)
ATGGAGTTTGGGCTGAGCTGGCTTTTTCTTGTGGCTATTTTAAAAGGTGTCCAGT
GCGGAGGCGGAGGAGGCGGATCCGATATCCAAATGACGCAAACTACGTCTATCC
TGTCTGCCTCCTTGGGCGACCGGGTGACGATTACGTGCCGGGCATCTCAAGATAT
TAGTAACTATCTTAACTGGTATCAACAGAACCCTGACGGAACGGTGAAATTGCTC
ATTTACTACACTTCTAGACTTCATAGTGGCGTGCCGTCCAGATTTTCCGGAAGTG
GGTCAGGTACAGAATACTCACTGACTATTAAGAACCTGGAACAAGAGGACATAG
CCACATATTTTTGTCAGCAAGGTAATATGGTCCCTTTTACCTTCGGCAGTGGTACT
AAGCTCGAAATAAAGGGAGGAGGGGGTAGCGGAGGTGGCGGCTCAGGCGGCGG
CGGCAGTCAGGTTCAACTTAAGGAATCCGGTCCCGGTCTTGTAGCGCCAAGTCA
GTCTCTCTCTATCACTTGTACGGTATCCGGGTTCTCCCTTACGTCCTACGGGGTAC
ACTGGGTTCGACAACCACCCGGAAAGGGCCTGGAGTGGTTGGGCGTCATATGGG
CAAGCGGAACTACGGATTATAACTCTGCCCTTATGTCTCGCCTCACCATTTCTAA
AGATAATAGTAAAAGCCAGGTTTTTCTTCGCATGAACTCTCTCCAAACTGATGAC
ACAGCAATGTACTACTGCGCCAGGGAGACTACAGCGAGTTTCGGTTATTGGGGC
TTGGGCACACTGGTCACAGTTTCAGCAGCTAGCACCACGACGCCAGCGCCGCGA
CCACCAACACCGGCGCCCACCATCGCGTCGCAGCCCCTGTCCCTGCGCCCAGAG
GCGTGCCGGCCAGCGGCGGGGGGCGCAGTGCACACGAGGGGGCTGGACTTCGC
CTGTGATTCCGGAATCTACATCTGGGCCCCTCTGGCCGGCACCTGTGGCGTGCTG
CTGCTGTCCCTGGTCATCACCCTGTACTGCAAGCGGGGCAGAAAGAAGCTGCTG
TACATCTTCAAGCAGCCCTTCATGCGGCCTGTGCAGACCACACAGGAAGAGGAC
GGCTGTAGCTGTAGATTCCCCGAGGAAGAGGAAGGCGGCTGCGAGCTGAGAGTG
AAGTTCAGCAGAAGCGCCGACGCCCCTGCCTATCAGCAGGGCCAGAACCAGCTG
TACAACGAGCTGAACCTGGGCAGACGGGAGGAATACGACGTGCTGGACAAGAG
AAGAGGCCGGGACCCTGAGATGGGCGGCAAGCCCAGACGGAAGAACCCCCAGG
AAGGCCTGTATAACGAACTGCAGAAAGACAAGATGGCCGAGGCCTACAGCGAG
ATCGGCATGAAGGGCGAGCGGAGAAGAGGCAAGGGCCATGACGGCCTGTACCA
GGGCCTGAGCACCGCCACCAAGGACACCTACGACGCCCTGCACATGCAGGCCCT
GCCTCCAAGATGA
CD19G5bbz DNA sequence:
(SEQ ID NO: 8)
ATGGAGTTTGGGCTGAGCTGGCTTTTTCTTGTGGCTATTTTAAAAGGTGTCCAGT
GCGGAGGCGGAGGAGGCGGATCCGACATTCAAATGACTCAAACAACATCCTCAC
TTTCTGCCTCACTCGGAGATCGCGTCACTATATCATGCAGGGCTTCCCAAGATAT
TAGCAAGTATCTGAACTGGTATCAGCAAAAACCGGATGGAACGGTTAAACTCTT
GATTTACCACACGTCCCGCCTCCATTCAGGCGTTCCCAGCAGATTTAGCGGCTCA
GGCTCTGGTACCGATTATTCACTTACTATATCCAATTTGGAACAGGAAGACATAG
CTACCTATTTCTGTCAACAAGGGAATACGCTTCCGTACACATTTGGTGGGGGTAC
TAAGCTGGAGATTACAGGCGGAGGAGGTTCTGGCGGCGGAGGGTCAGGGGGCG
GTGGTTCAGAGGTGAAGCTGCAAGAGTCAGGTCCGGGATTGGTAGCTCCGAGTC
AAAGCTTGTCCGTGACATGCACCGTGTCAGGTGTCAGCCTCCCAGACTACGGTGT
GTCCTGGATCAGACAACCTCCTCGGAAGGGGTTGGAGTGGCTCGGTGTGATTTG
GGGGTCAGAGACAACCTACTACAACTCAGCACTCAAGAGTCGCCTGACAATTAT
TAAAGACAACAGCAAAAGTCAAGTCTTCCTCAAGATGAACAGCCTGCAGACTGA
CGATACCGCCATATATTATTGTGCGAAGCACTATTACTACGGAGGTTCCTATGCA
ATGGATTATTGGGGGCAAGGGACGAGTGTAACAGTCAGCAGTGCTAGCACCACG
ACGCCAGCGCCGCGACCACCAACACCGGCGCCCACCATCGCGTCGCAGCCCCTG
TCCCTGCGCCCAGAGGCGTGCCGGCCAGCGGGGGGGGCGCAGTGCACACGAG
GGGGCTGGACTTCGCCTGTGATTCCGGAATCTACATCTGGGCCCCTCTGGCCGGC
ACCTGTGGCGTGCTGCTGCTGTCCCTGGTCATCACCCTGTACTGCAAGCGGGGCA
GAAAGAAGCTGCTGTACATCTTCAAGCAGCCCTTCATGCGGCCTGTGCAGACCA
CACAGGAAGAGGACGGCTGTAGCTGTAGATTCCCCGAGGAAGAGGAAGGCGGC
TGCGAGCTGAGAGTGAAGTTCAGCAGAAGCGCCGACGCCCCTGCCTATCAGCAG
GGCCAGAACCAGCTGTACAACGAGCTGAACCTGGGCAGACGGGAGGAATACGA
CGTGCTGGACAAGAGAAGAGGCCGGGACCCTGAGATGGGCGGCAAGCCCAGAC
GGAAGAACCCCCAGGAAGGCCTGTATAACGAACTGCAGAAAGACAAGATGGCC
GAGGCCTACAGCGAGATCGGCATGAAGGGCGAGCGGAGAAGAGGCAAGGGCCA
TGACGGCCTGTACCAGGGCCTGAGCACCGCCACCAAGGACACCTACGACGCCCT
GCACATGCAGGCCCTGCCTCCAAGATGA

The results of the experiments are now described.

### Example 1

Long-term persistence of CAR T cells is associated with superior outcomes and credited to the formation of long-lived memory CAR T cells that afford continuous immunosurveillance. Despite the remarkable efficacy of CAR therapy, only a minority of patients achieve long-term remission, highlighting the unmet need to understand cellular mechanisms of memory CAR T cell formation. In mouse models of infection and ovalbumin immunization, asymmetric T cell division (ATCD) has been observed after T cell activation. The daughter cell closer to the antigen presenting cell, i.e. proximal daughter cell, inherits the immunologic synapse, exhibits a moderate increase in CD8 surface positivity, and is more likely to differentiate into a short-lived effector T cell. The distal daughter cell, conversely, remains less differentiated and becomes a long-lived memory cell with distinct transcriptional and metabolic profile (Chang, J. T. et al. (2007) Science 315, 1687-1691, doi:doi:10.1126/science.1139393; Verbist, K. C. et al. (2016) Nature 532, 389-393, doi:10.1038/nature17442.)

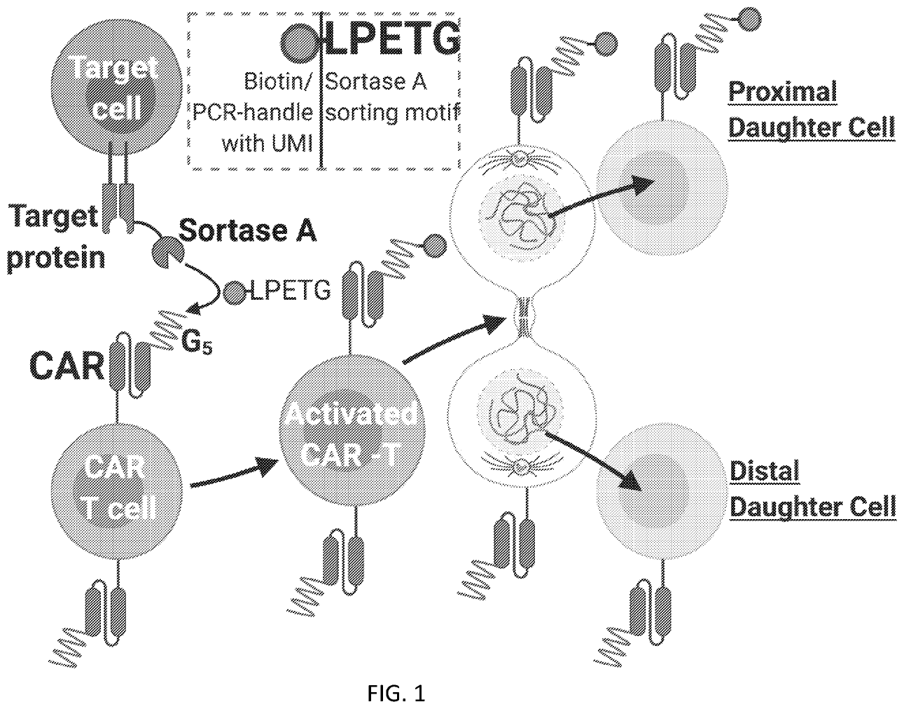

Since target recognition by CAR T cells is MHC- and therefore CD8 co-receptor-independent, it was reasoned that target-induced labeling of CAR molecules within the immunologic synapse could instead be used to distinguish and characterize first division proximal and distal CAR daughter cells. Therefore, an elegant approach that uses sortase A-mediated irreversible labeling of the ‘kiss and run’ between immune cells across immune synapses, a technique known as Labeling Immune Partnerships by SorTagging Intercellular Contacts (LIPSTIC) (Nature volume 553, 496-500(2018)), was optimized (FIG. 1). Other methods of labeling can similarly be employed (e.g. labeling by trogocytosis, or an α1,3-fucosyltransferase-based system that does not require genetic manipulation of T cells and affords similarly specific labeling using GDP-fucose-biotin, Liu Z, et al. (2020) Cell. 183(4):1117-1133.e19. doi:10.1016/j.cell.2020.09.048). Alternatively, entire first, second, third and fourth division daughter cell populations (separately or combined) can be isolated by dye dilution or other methods known in the arts (e.g. histone-mCherry dilution). Defined cell division populations can also be identified and isolated by a defined time period of stimulation.

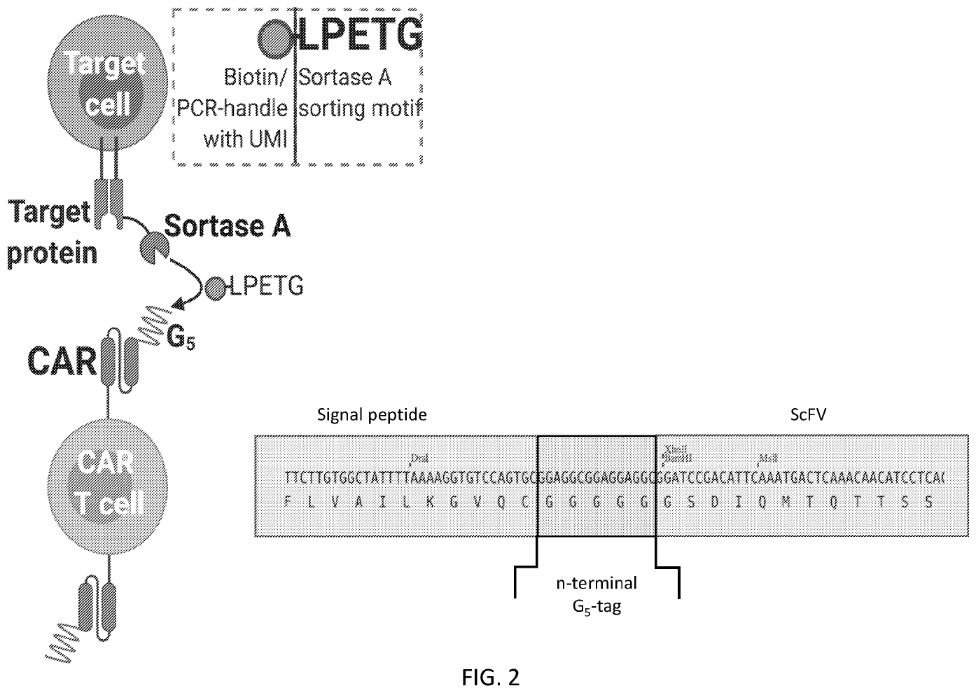

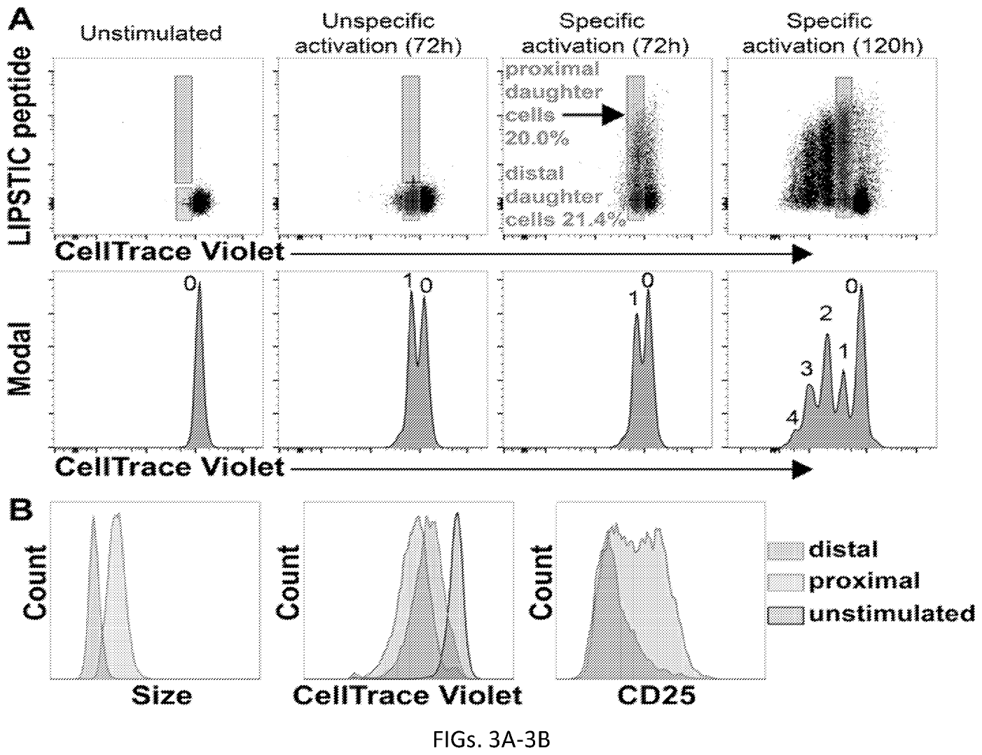

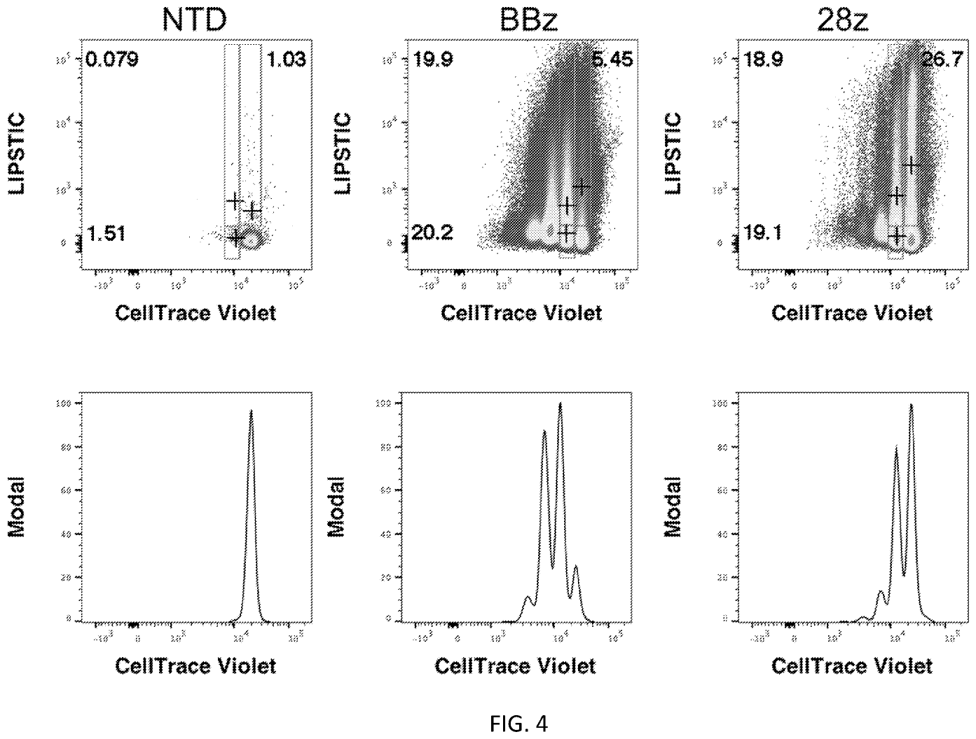

A minor modification was introduced to CAR molecules by appending an n-terminal penta-glycine (G5) tag, and the B-ALL leukemia line NALM6 was modified by expressing CD19 (after disruption of the endogenous CD19 locus) or other targets with an appended n-terminal sortase (FIG. 2). Consistent with previously reported features of proximal daughter cells, LIPSTIC+ first division daughter cells exhibited an increase in cell size, CD25, CD98, and subsequent proliferative pace compared to LIPSTIC− first division daughter cells, establishing that the first cell division of human CAR T cells after activation is asymmetric, and that LIPSTIC positive and negative daughter cells represent proximal and distal first division daughter cells, respectively (FIGS. 3A-3B).

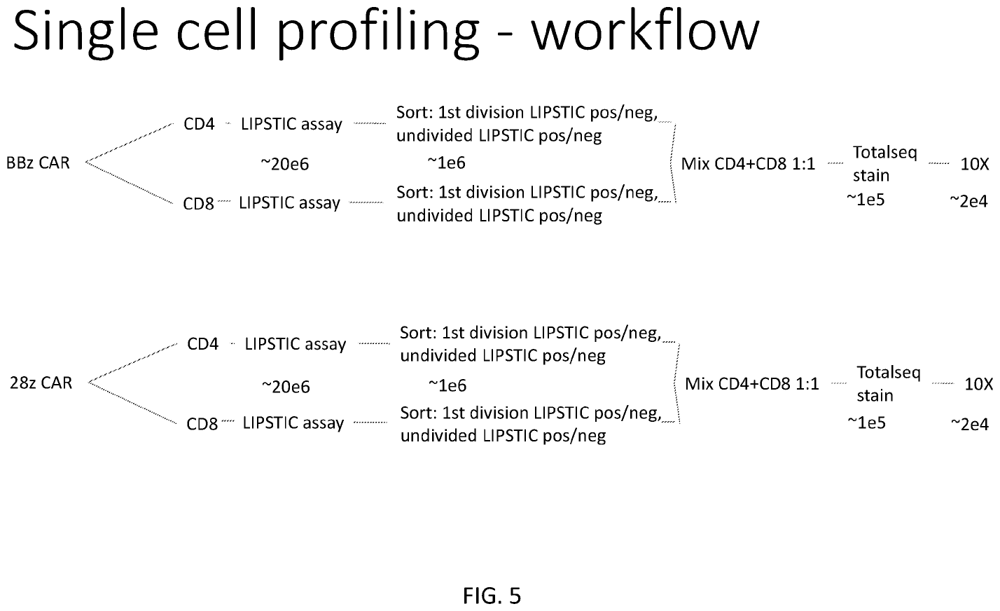

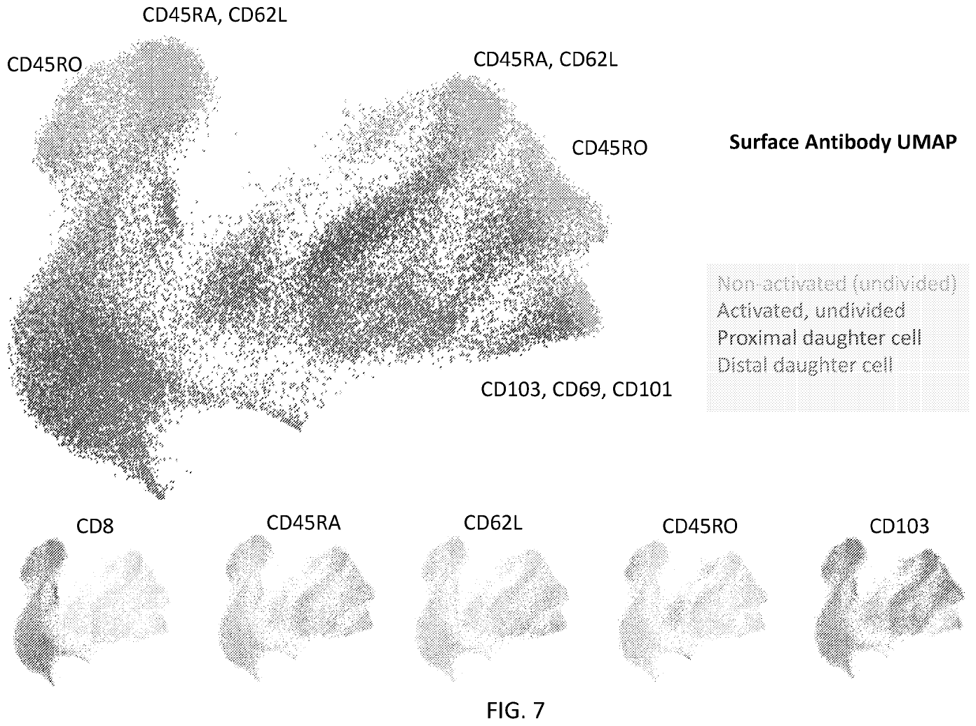

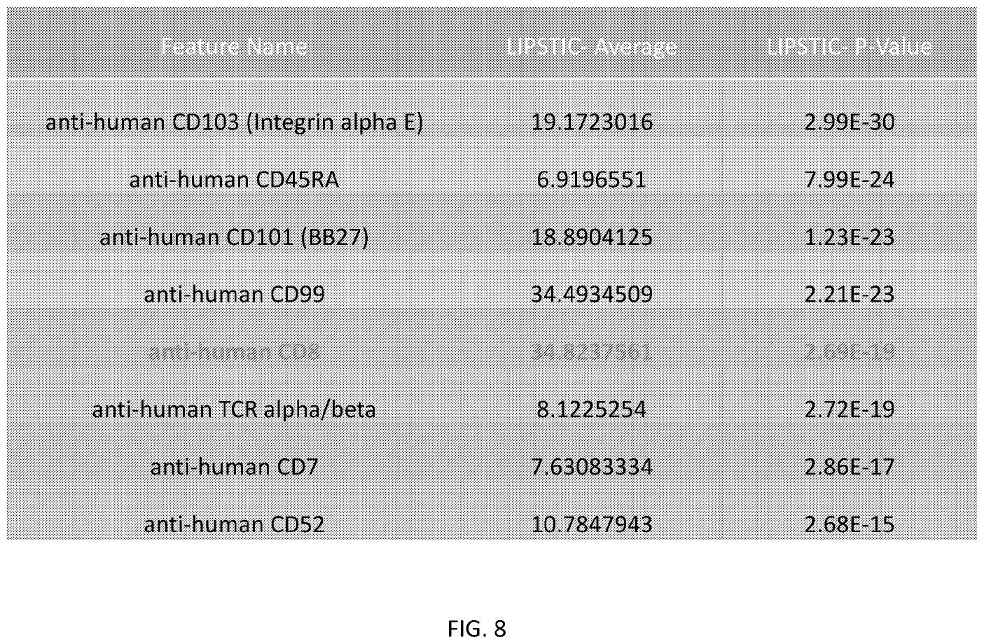

First division daughter cells, resting T cells, and activated T cells were stained before cell division with a custom DNA-barcoded antibody cocktail detecting 198 surface proteins followed by single cell droplet encapsulation and sequencing (FIG. 5). Dimensionality reduction with universal manifold and protection (UMAP) demonstrated that resting CAR T cells cluster into three populations driven by differential positivity for CD62L, CD45RA, CD45RO and CD103, consistent with different stages of T cell differentiation (FIG. 7). Activated CAR T cells prior to the first cell division, conversely, demonstrated a distinct phenotype while maintaining three subsets, reflecting the activation induced changes of the surface protein landscape. Of note, first division daughter cells occupied the space between resting and activated, undivided T cells with a clear distinction between proximal and distal first division daughter cells across different subsets, underscoring that activated CAR T cells establish global asymmetry of the cell surface proteome during the first cell division.

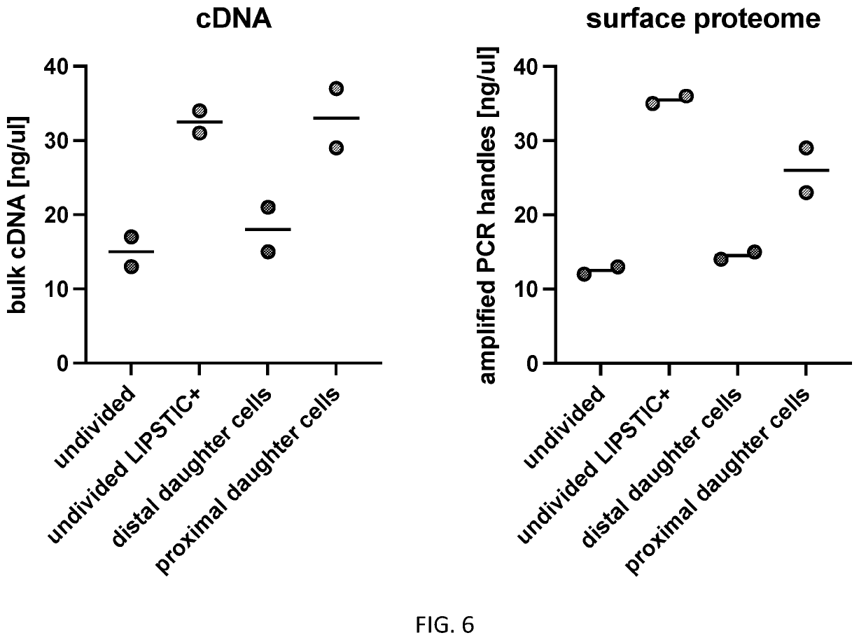

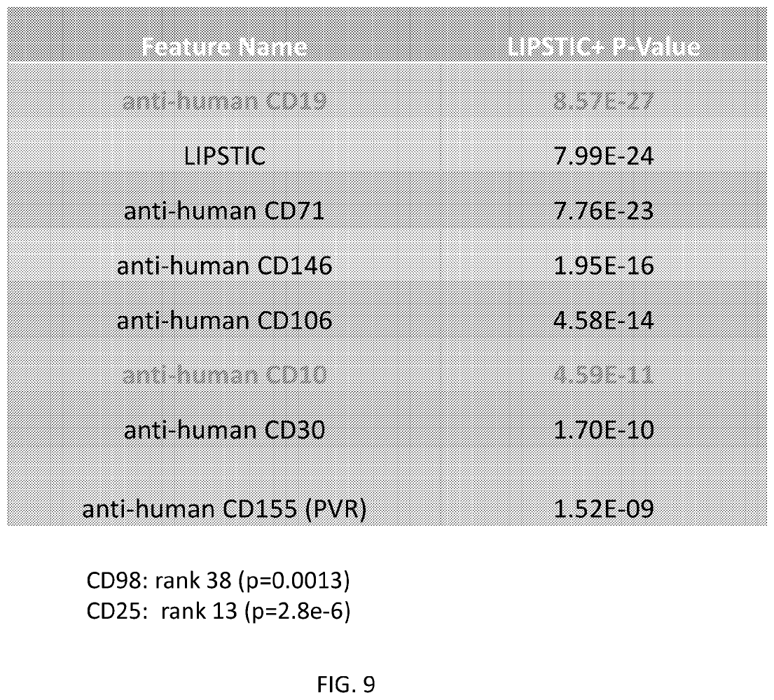

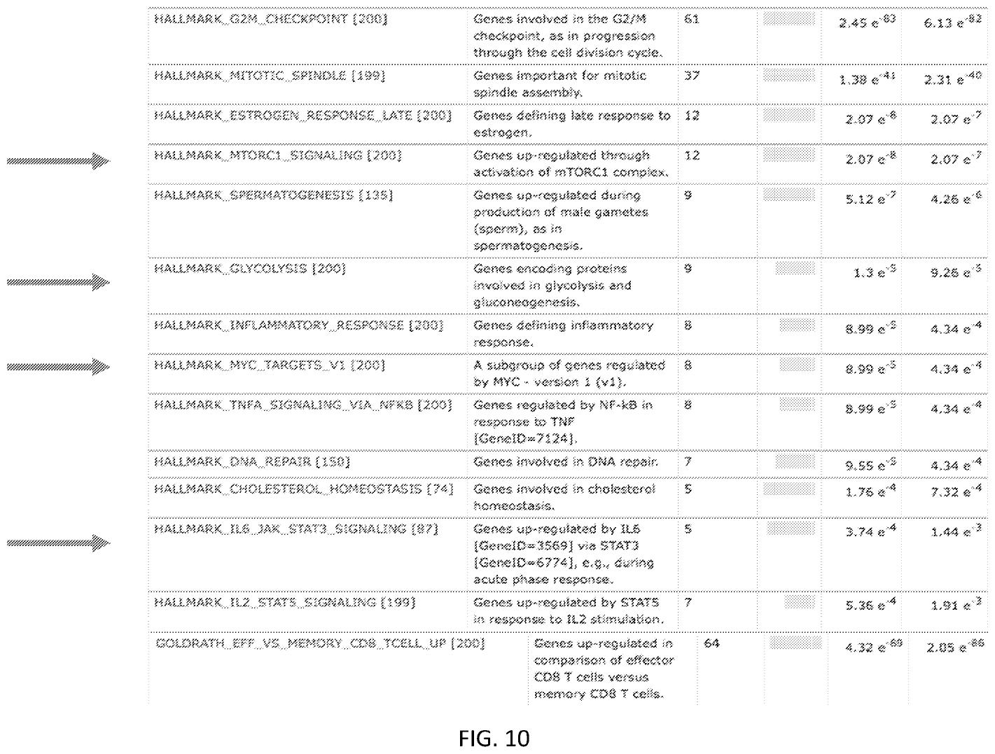

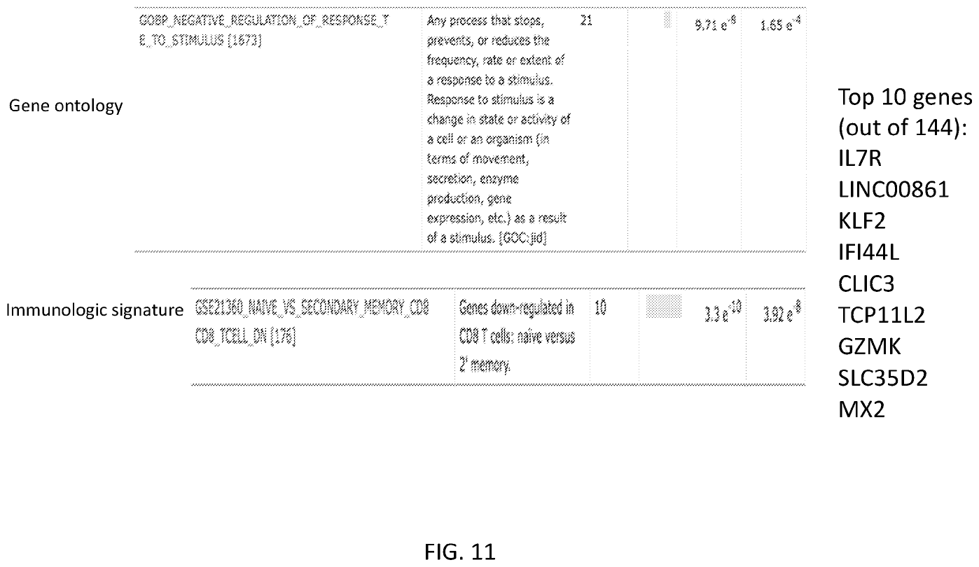

Pairwise comparison of proximal and distal daughter cells derived from naive T cells demonstrated increased surface positivity for CD45RA on distal daughter cells and for CD25 on proximal daughter cells, confirming the flow cytometry data (FIG. 6). Additionally, distal daughter cells demonstrated a notable increase in the endogenous TCR in addition to CD5, consistent with the previously reported anti-proliferative effect of CD5 in human T cells. Comparison of the single cell transcriptome demonstrated differential transcriptional programs in proximal and distal daughter cells. Notably, LEF1, TCF7, CCR7, IL7R and KLF2 demonstrated increased expression in distal daughter cells, suggesting that distal daughter cells continue to express genes associated with naïve T cells, whereas genes associated with activated, effector cells and glycolysis are enriched in the proximal daughter cells (FIGS. 9-12).

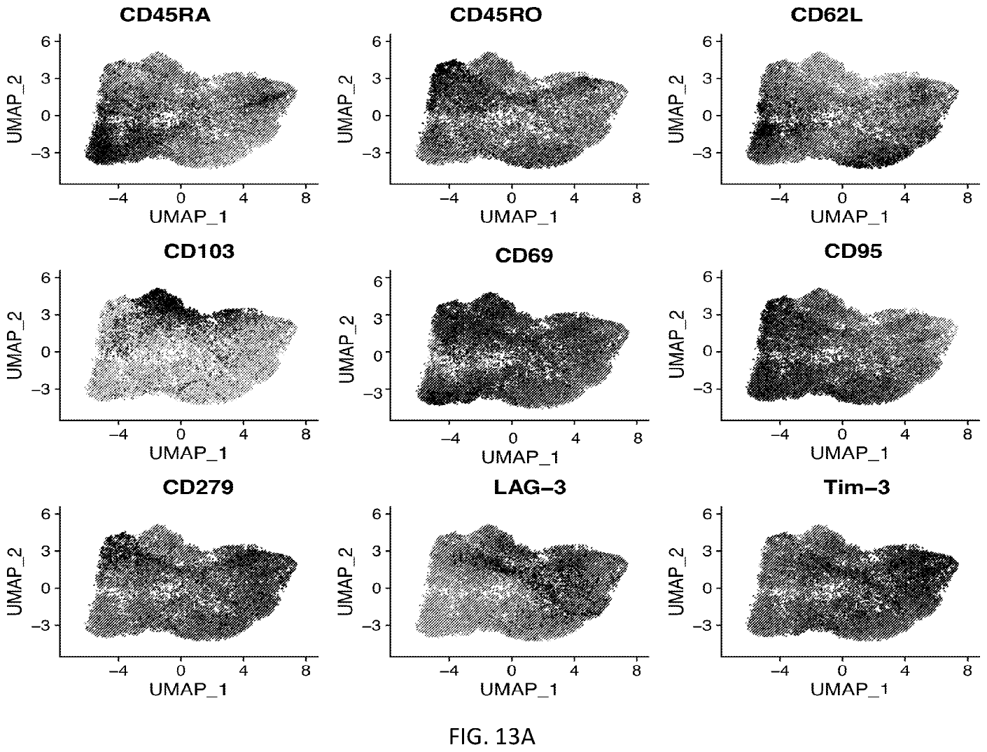

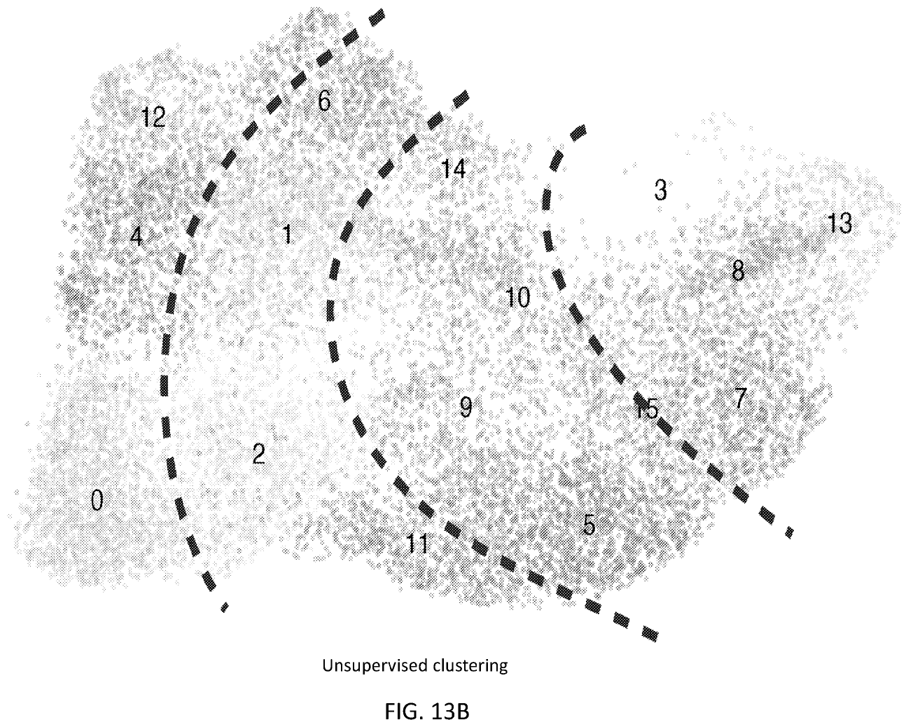

To test the functional relevance of surface proteome and transcriptome asymmetry in first division CAR T cells, the longevity and regenerative capacity of proximal and distal first division daughter cells in vivo was characterized (FIGS. 13A-13B). To this end, sorted proximal and distal daughter CD19 targeting CAR T cells were injected separately into NSG mice followed by injection of NALM6 leukemia cells 35 days later. Of note, a reduced CAR T cell dose that without in vitro restimulation is not sufficient for prolonged persistence of T cells, was deliberately injected. Prior to NALM6 injection, increased numbers of T cells were observed in mice that had received distal daughter cells compared to mice that had received proximal or unstimulated T cells, indicating that distal daughter cells exhibited increased regenerative capacity compared to proximal daughter cells and persisted beyond homeostatic proliferation in immunodeficient NSG mice. After NALM6 challenge, only distal daughter cells were able to control the leukemia, while mice that had received proximal daughter cells demonstrated an outgrowth of leukemia cells. Analysis of bone marrow, spleen and blood confirmed the presence of T cells for at least 2 months after NALM6 injections (i.e. at least 3 months after T cell injection) in mice that had received distal daughter cells. Conversely, mice that had received proximal daughter cells demonstrated reduced numbers or absence of T cells in these compartments, establishing distal daughter cells functionally as precursors of long-lived memory T cells.

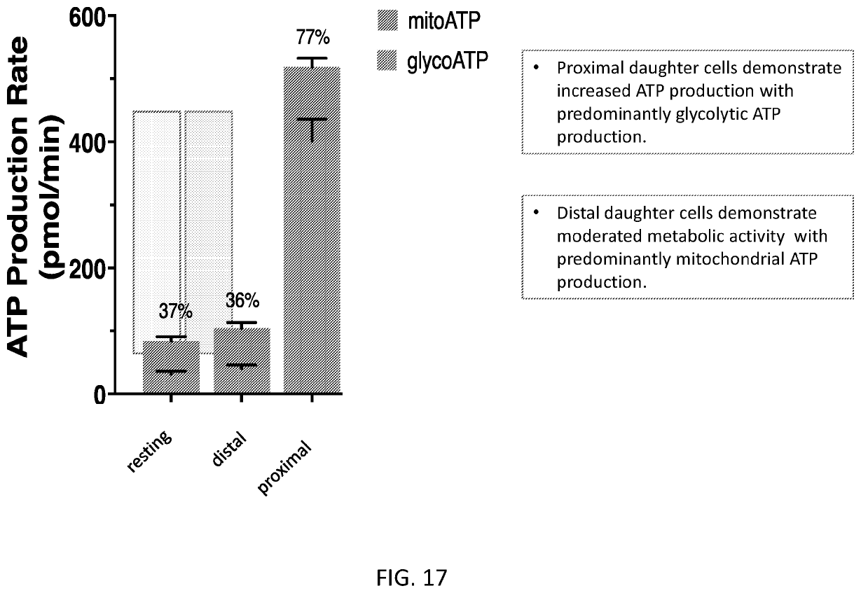

Since longevity in T cells has been linked to specific metabolic programs, the metabolic profile of sorted proximal and distal daughter cells was compared (FIG. 17). The daughter cell metabolism was characterized by quantifying the extracellular acidification rate (ECAR) and oxygen consumption rate (OCR) under conditions of mitochondrial stress. Proximal demonstrated an overall increased metabolic activity compared to distal daughter cells with the great majority of ATP being produced in glycolysis. In contrast, distal daughter cells exhibited only a minor increase in metabolic activity compared to resting cells; their ATP, however, was predominantly produced during oxidative phosphorylation. Consistent with this observation, distal daughter cells at baseline and under stress showed an increased OCAR/ECAR ratio compared to proximal daughter and resting T cells, confirming that metabolic asymmetry was established in activated human CAR T cells during the first cell division and correlated with in vivo longevity of distal daughter cells.

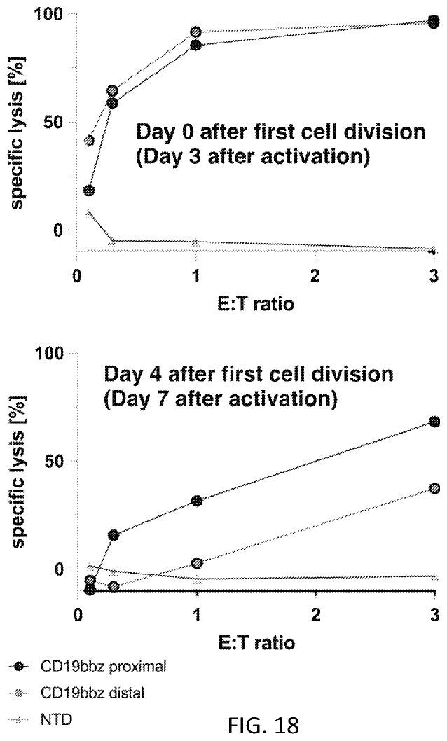

Given these metabolic differences and their implications for differentiation and effector function, the cytotoxic capacity of first division daughter cells in vitro was explored. As expected, proximal daughter cells demonstrated exquisite cytotoxic potency with substantial killing at very low effector:target ratios that are not sufficient for detectable cytotoxicity in resting CAR T cells (FIG. 18). This increased cytotoxic potential in proximal daughter cells was observed on day 1 and on day 5 after cytogenesis (day 3 and 7 after activation), consistent with the effector differentiation of proximal daughter cells. Surprisingly, however, the in vitro cytotoxic function of distal daughter cells was indistinguishable from proximal daughter cells on day 1 and 2 after cytogenesis, indicating that distal daughter cells despite their predominant OXPHOS metabolism and transcriptional memory precursor phenotype enter a state of heightened target sensitivity after cytogenesis. To test the durability of this state of ‘target readiness’, distal daughter cell cytotoxicity was tested on day 5 after cytogenesis (day 7 after activation) and a marked decrease in cytotoxicity compared to proximal daughter cells was observed. Thus, it was concluded that distal daughter cells, despite being transcriptionally predestined to become memory cells, cycle through a transitional state of effector function.

Next, it was tested whether distal CAR daughter cells could exhibit effector-like functions in vivo (FIG. 21). Given the increased cytotoxic potential of first division daughter cells in vitro, it was reasoned that both proximal and distal daughter cells would rapidly eliminate leukemia cells, precluding us from observing differences in their longevity. A previously established stress-test model was used in which insufficient numbers of CAR T cells are injected into leukemia-bearing mice. Consistent with in vitro assays, a rapid but transient decline of leukemia burden was observed after injection of proximal daughter cells confirming their robust cytoxicity and short-lived functional phenotype. Importantly, mice that were treated with distal daughter cells exhibited a similar initial decline in leukemia burden as mice treated with proximal daughter cells, confirming that they at least initially exhibit similar cytotoxic potential. In contrast to proximal daughter cells, however, distal daughter cells showed long-term control of leukemia, providing evidence that distal daughter cells transiently exhibit effector functions in vivo and that target-engagement during this stage does not decrease their longevity and replicative potential.

The methods herein could be applied to optimize the therapeutic function of donor lymphocytes, tumor infiltrating lymphocytes, or genetically-engineered T cells including T cell receptor (TCR) T cells, chimeric antigen receptor (CAR) T cells, and chimeric autoantibody receptor (CAAR) T cells that are in clinical use or evaluation for a broad range of inflammatory, fibrotic, infectious, oncologic, or immune-mediated conditions.

In conclusion, LIPSTIC labeling allows distinction of proximal and distal CAR daughter cells after the first cell division. Human CAR T cells undergo asymmetric cell division upon activation with distinct surface proteome, transcriptional program, (metabolism), and functional properties of proximal and distal daughter cells. Proximal 1st division daughter cells assume an effector-like phenotype with rapid proliferation, and an expression profile consistent with effector differentiation. Distal 1st division daughter cells activate a transcriptional program that restrains activation, glycolytic energy production, proliferation and effector T cell differentiation. Collectively, these studies establish asymmetric cell division as a novel framework for understanding mechanisms of CAR-T cell differentiation and influencing therapeutic outcomes.

### Example 2

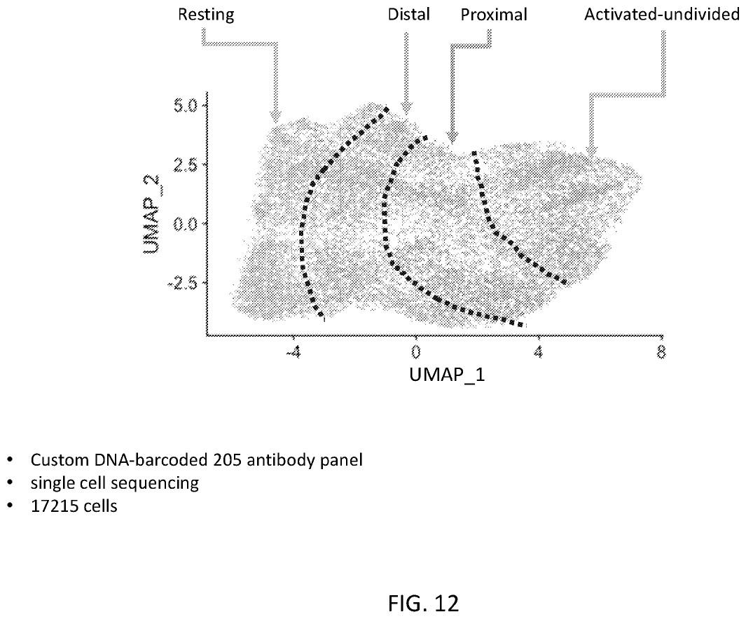

Methods: The following methods describe the experimental procedures performed to generate the data shown in FIGS. 12-15. Biotinylated LPETG peptide (biotin-aminohexanoic acid-LPETGS, C-terminal amide, 95% purity) were purchased from LifeTein (custom synthesis), reconstituted in PBS at 10 mM and stored at −80° C.

To label target cells, Nalm6 cells (expressing sortase-tethered target molecules) were incubated with biotinylated LPETG peptide (100 μM, LifeTein) for 30 minutes at 37° C. in RPMI/10% FBS, followed by washing three times to remove excess soluble peptide. Sortase-bound LPETG was then labeled with fluorescent streptavidin (PE, AF647 or APC; 10 μg/mL; BioLegend) for 30 minutes at 37° C. Cells were then washed three times and resuspended at 1×10⁶ cells per mL.

LIPSTIC assays were performed using fully rested T cells that had not demonstrated cell number increases in ˜2 days. For the CARs in the presented studies, the transduction efficiencies were between 20-85%, and the cell size of rested T cells was between 200 and 260 fL, which was achieved 12-15 days after activation. CAR and control T cells (non-transduced or transduced with irrelevant CAR) were washed once with PBS and resuspended in PBS at a concentration of 1×10⁷ cells per mL, incubated at 37° C. for 10 minutes with CellTrace Violet (CTV, final concentration 0.5 μM, which is 10-fold lower than the manufacturer's recommendation), washed three times with RPMI/10% FBS and resuspended in RPMI/10% FBS at a concentration of 1×10⁶ cells per mL. Target cells and CAR T cells were mixed in a 6-well plate well in a total volume of 6 mL (5×10⁶ effector cells to 1×10⁶ labelled target cells). Cells were incubated for 72 hours prior to cell sorting (BD Biosciences AriaII) and subsequent analysis of first-division daughter cells.

Sorting gates were established for live single cells that were negative for GFP (excluding target cells), positive for CTV (zero or first cell division) and positive or negative for LPETG. LPETG positivity was determined relative to untransduced T cells, CAR T cells incubated without target cells or irrelevant CAR T cells incubated with target cells (threshold for LPETG positivity was generally the same for all controls). Cells were sorted into RPMI/10% FBS at 4° C. on a BD Biosciences FACS Aria II sorter (100 μm nozzle, 20 psi) prior to downstream analysis.

In two independent experiments, first-division proximal, first division distal, activated-undivided, and resting CD8 CAR T-cells (1.5×10⁵ cells each) each from two separate healthy donors, sorted as described above from the LIPSTIC assay, were separately incubated in flow cytometry staining buffer (BioLegend) with a custom TotalSeq-C antibody cocktail in 100 μl for 30 minutes at 4° C. prior to washing three times. Live cells were counted by trypan blue dye exclusion, and cell concentration was adjusted to 1.5×10⁶ cells per mL. For clone 5A6.E9, ˜10000 live CD8 T-cells from each LIPSTIC population (i.e. proximal, distal, resting, activated-undivided) were each loaded onto NextGem K chips (10× Genomics) and processed in a 10× Chromium device according to the manufacturer's recommendations. For clone FMC63, ˜1250 live CD8 T-cells of each LIPSTIC population were separately stained with the custom TotalSeq-C antibody cocktail with anti-human hashtag antibodies, loaded onto one NextGem K chip, and processed. Library preparation was performed according to the 10×5′ V2 protocol for antibody-derived tags (ADT), gene expression (GEX) and paired alpha and beta TCR chains (VDJ). cDNA and subsequent library intermediates were checked for correct size, appropriate quantity, and quality with a DNA high-sensitivity kit on a Bioanalyzer 2100 (Agilent). Libraries were sequenced in paired-end dual-index mode for 150×2 cycles on a NovaSeq 6000 sequencer (Illumina, 1 lane of a S4 cartridge). All cells in each experiment were sorted and stained on the same day and libraries were processed in parallel and sequenced in the same lane to minimize batch effects. Counts for demultiplexed GEX, ADT and TCR libraries were obtained with the STAR method of the Cell Ranger multi pipeline (10× Genomics, Cell Ranger v6.1.2) using the human GENCODE v32/Ensembl 98 GRCh38 reference, which then were aggregated with the Cell Ranger aggr pipeline with read depth normalization to further reduce batch effects across libraries. Downstream analysis was performed with the Seurat V4 R package. Cells with more than 25% mitochondrial (to account for activation-induced increase of mitochondrial gene transcription) and less than 7.5% ribosomal gene transcripts were excluded, and doublets and low-quality cells were further eliminated by limiting analysis to cells with a transcript count between 500 and 40000 and a minimum number of detected genes of 500. Counts were single cell transformed using the sctransform V2 and glmGamPoi packages. Dimensionality reduction was performed based on ADT counts with subsequent analysis of genes and surface proteins of interest and differentially expressed genes/surface proteins for T_{N}, T_{CM}, T_{EM} and T_{RM} subsets.

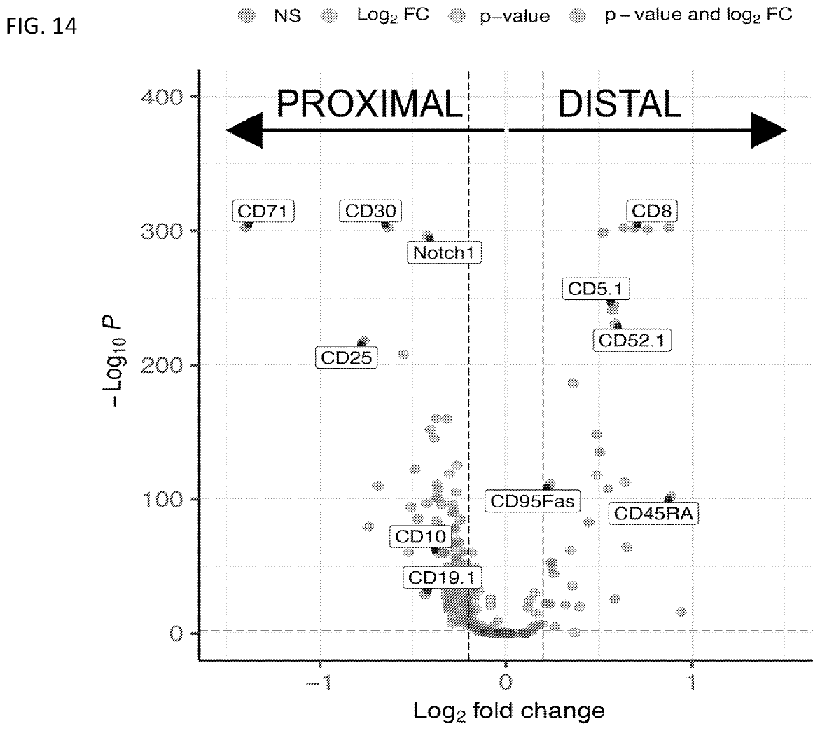

Results: Asymmetric cell division in human CAR T cells exhibited unique features (FIG. 14). Differentially expressed gene analysis was performed comparing the surface antibody positivity of clusters 2 and 9 from FIG. 13B. Differential antibody positivity between proximal and distal first division daughter cells is depicted in FIG. 14. Selected antibody targets are displayed. Of note, surface CD8 is increased on distal daughter cells compared to proximal daughter cells (FIG. 14), which differs from previous reports of T cells that had been stimulated through their endogenous T cell receptor. This result was reproducible for CAR T cell targeting different antigens (e.g. CD19, gamma-delta TCR).

Genes differentially expressed in distal first division daughter cells included: CD103 (Integrin alpha E), CD45RA, CD99.1, TCRalpha/beta, CD101 (BB27), CD8, CD49a, CD7.1, CD48.1, CD52.1, CD73, CD5.1, CD3, CD224, CD45, CD47.1, CD11a, CD18, CD31, CD27.1, mouse CD49f, CD2.1, CD26, CD195 (CCR5), CD38.1, CD244 (2B4), CD29, CD314 (NKG2D), CD305 (LAIR1), CD352 (NTB-A), CD49d, CD95Fas, CD69.1, integrin beta 7, CD44.1, CD273B7 (DCPD-L2), CD45RO, CD96 (TACTILE), CD57 Recombinant, CD94, CD56, CD49b, CD226 (DNAM-1), CD278 (ICOS), CD127 (IL-7R), CLEC12A.1, CD39, CD161, HLA-DR, CD81 (TAPA-1), HLA-E.1, HLA-ABC, CD66ace, and CD28.1.

Genes differentially expressed in proximal first division daughter cells included: CD30, CD71, CD25, CD106, CD117 (c-kit), CD88 (C5aR), CDllc, CD155 (PVR), CD223 (LAG-3), CD146, CD163.1, CD19.1, CD194 (CCR4), CD23, Notch1, CD105, CD169 (Sialoadhesin Siglec-1), CD83.1, aCD207, CD137 (4-1BB), TCRdeltagamma, CD134 (OX40), B7-H4, CD324E (Cadherin), CD122 (IL-2R beta), CD10, CD206 (MMR), CD178 (Fas-L), CD82.1, CD141 (Thrombomodulin), CD80.1, LOX-1, CD1a, IgGFc, CD123, TCRValpha24, CD209 (DC-SIGN), CD272 (BTLA), CD304 (Neuropilin-1), CD85 (jILT2), CD252 (OX40L), CD303 (BDCA2), IgM, TSLPRTSLP-R, CD98, CD34.1, CD20, CD235ab, CD62L, CD144VE (Cadherin), CD307d (FcRL4), CD197 (CCR7), CD201 (EPCR), CD54, CX3CR1.1, CD360 (IL-21R), CD140b (PDGFRbeta), CD112 (Nectin-2), CD124 (IL-4Ralpha), CD257 (BAFFBLYS), CD335 (NKp46), CD152 (CTLA-4), EGFR.1, GARPLRRC32, CD62E, CD269 (BCMA), CD158 (KIR2DL1S1S3S5), Podoplanin, XCR1.1, CD70.1, CD254 (TRANCERANKL), Podocalyxin, CD158f (KIR2DL5), CD274 (B7-H1 PD-L1), CD66b, CD21, CD307 (eFcRL5), CD268 (BAFF-R), CD154, CD137L (4-1BB Ligand), CD119 (IFN gamma R alpha chain), CD1d, CD370 (CLEC9ADNGR1), CD267 (TACI), CD107a (LAMP-1), CD24.1, CD13, TCRVgamma9, CD357 (GITR), TCRVdelta2, Notch3, CD40.1, CD326 (Ep-CAM), CD204, Fc epsilon RI alpha, CD294 (CRTH2), CD158el (KIR3DL1, NKB1), CD150 (SLAM), CD14.1, Ig light chain kappa, LIPSTIC1, CD184 (CXCR4), CD196 (CCR6), CD79b (IgB), CD16, DR3TRAMP, CD319 (CRACC), CD258 (LIGHT), CD32, TCRVbeta131, CD275 (B7-H2, ICOSL), CD45R/B220, CD279, CD35, CD42b, CD366 (Tim-3), CD336 (NKp44), CD140a (PDGFRalpha), IgD, CD62P (P-Selectin), CD1c, CD41, CD11b, CD185 (CXCR5), CD22.1, CD328 (Siglec-7), CD325 (N-Cadherin), CD86.1, CD36.1, CD158b (KIR2DL2/L3, NKAT2, KLRG1, MAFA), Ig light chain lambda, GPR56, TIGIT/VSTM3, CD337 (NKp30), CD64, CD33.1, TCRValpha72, CD270 (HVEMTR2), CD4.1, HLAA2, CD183 (CXCR3), and CD58 (LFA-3).

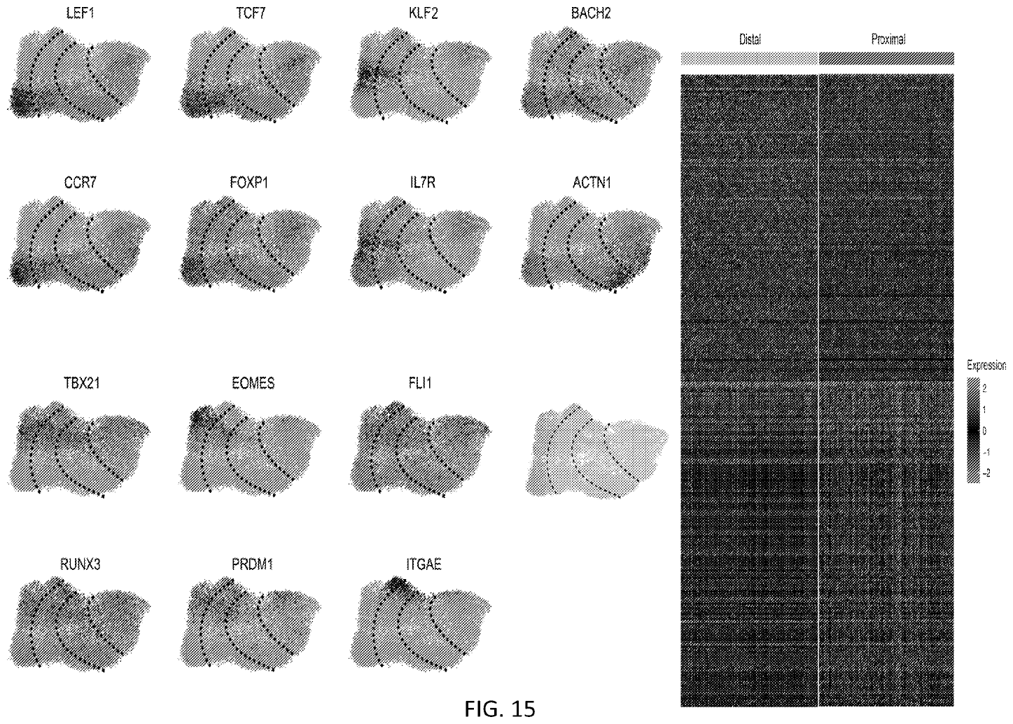

CAR T cells asymmetrically sort fate-associated transcripts (FIG. 15). Uniform manifold approximation and projection (UMAP) of 17215 single cells displaying selected gene expression levels. UMAP projection was performed as detailed herein and the expression level for selected genes for each cell is displayed in a color-coded format with light grey indicating low positivity and dark grey/black indicating high positivity (FIG. 15). Dotted lines represent the border between proximal, distal, resting and activated-undivided cells. Name of respective gene displayed at the top of each panel. Selected genes represent canonical transcription factors or markers associated with naïve/memory, effector or tissue-resident T cells. FIG. 15, right panel: heat map comparing proximal and distal first division daughter cells. Each line represents one gene, each column represents one cell, colors indicated gene expression level as shown in the legend (log-fold change). The top 150 differentially expressed genes of proximal and distal daughter cells. This analysis demonstrates that the gene expression profile of proximal and distal daughter cells differs globally and that fate-associated transcripts are asymmetrically sorted during the first cell division after activation.

Conclusions: Results from these experiments provide valuable insights into the asymmetric sorting of fate-associated transcripts in CAR T cells during the first cell division after activation. The global differences in gene expression profiles between proximal and distal daughter cells demonstrates that these cells adopt distinct functional fates. This understanding of the underlying mechanisms governing CAR T cell behavior contributes to the development of more effective immunotherapies, enabling better control of cell fate decisions and improved therapeutic outcomes in the treatment of various diseases, including cancer.

### Example 3: Distal First Division Daughter Cells Demonstrate In Vivo Longevity and Leukemia Control

Methods: Immunodeficient NOD-scid-gamma (NSG; NOD.Cg-Prkdcscid Il2rgtm1Wjl/SzJ) mice were bred in house under an approved Institutional Animal Care and Use Committee (IACUC) protocol and maintained under pathogen-free conditions. To facilitate engraftment of T cells, bulk (CD4+ and CD8+) T cells were used in in vivo studies.

For functional longevity studies, 2.5×10⁵ proximal or distal CAR daughter cells, non-activated resting CAR T-cells, or non-transduced T-cells were intravenously injected into NSG mice on day 0.35 days later, mice were challenged with 1×10⁶ Nalm6 cells. Leukemia burden was determined by bioluminescence imaging. Bioluminescence was quantified with an IVIS Lumina III (PerkinElmer) 2-3 times per week after Nalm6 injection. To do so, 150 mg/kg D-luciferin potassium salt (Gold Bio) was injected intraperitoneally. Mice were anaesthetized with 2% isoflurane, and luminescence was assessed 10 minutes after injection in automatic exposure mode. Total flux was quantified using Living Image 4.4 (PerkinElmer) by drawing rectangles of identical area around mice reaching from head to the 50% of the tail length; background bioluminescence was subtracted for each image individually.

In the stress test model, NSG mice were injected with 1×10⁶ Nalm6 cells on day 0. Engraftment of Nalm6 was confirmed on day 3 by bioluminescence imaging. On day 4, mice were treated with 2.5×10⁵ proximal or distal daughter CAR T-cells or 2.5×10⁶ nonactivated resting CART or nontransduced T-cells by intravenous injection. Leukemia burden was determined with bioluminescence imaging as above. Mice were sacrificed when they had reached a total bioluminescence flux of at least 5×10⁹ photons per second for 3-5 days, demonstrating loss of leukemia control.

Peripheral blood was obtained by retro-orbital bleeding. Mice were euthanized for organ harvest according to local IACUC guidelines, and spleen and blood samples were assessed by flow cytometry as described herein.

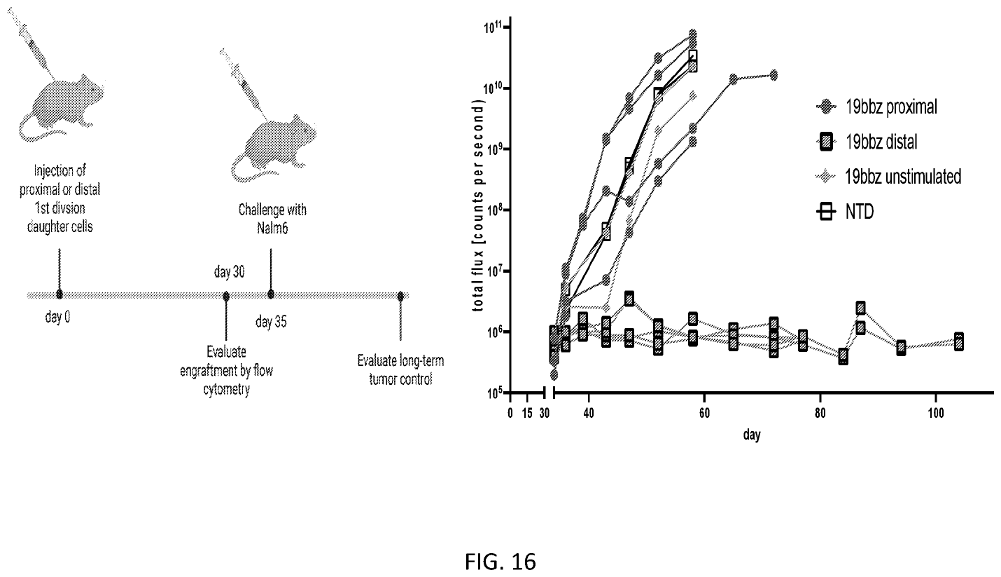

Results: NSG mice were injected on day 0 with proximal or distal daughter cells (2.5e5 cells per mouse) or control cells (same number of cells, nontransduced T cells from the same donor, unstimulated CAR T cells from the same donor) (FIG. 16, left panel). T cell numbers were evaluated on day 30 by flow cytometry and mice were injected on day 35 with 1e6 NALM6 leukemia cells to functionally challenge injected T cells and assess their longevity. All injections were performed intravenously. Quantification of leukemia burden/rejection by in vivo bioluminescent quantification of NALM6 cells is shown in FIG. 16, right panel. NALM6 cells express click-beetle-green luciferase whose activity is used to quantify leukemia burden on indicated days. Y-axis displays bioluminescent signal in photons per second, x-axis displays time in days. Each line represents one mouse. Data representative of 2 separate experiments using T cells from 2 different healthy human donors. Longevity and superior leukemia control was demonstrated by distal first division daughter cells.

Conclusions: Distal daughter cells from the first division display enhanced longevity and functionality in vivo, as evidenced by their remarkable capacity to eliminate leukemia cells injected 30 days after the T cell infusion. This finding highlights the superior persistence and therapeutic potential of distal daughter cells in vivo.

### Example 4: Distal Daughter Cells Demonstrate Muted Metabolic Activity with Preferential Mitochondrial ATP Production

Methods: To determine daughter cell metabolism, sorted proximal and distal daughter cells were subjected to a Seahorse mitochondrial stress test (Agilent Technologies). Individual wells of an XF96 cell-culture microplate were coated with CellTak as per the manufacturer's instructions. The matrix was adsorbed overnight at 37° C., aspirated, air-dried, and stored at 4° C. until use. Mitochondrial function was assessed on day 0 or day 1 after sorting proximal/distal or undivided cells. T cells were resuspended in non-buffered RPMI 1640 medium containing 5.5 mM glucose, 2 mM L-glutamine, and 1 mM sodium pyruvate and seeded at 1.5×10⁵ cells per well. The microplate was centrifuged at 1,000×g for 5 minutes and incubated in standard culture conditions for 60 minutes. During instrument calibration (30 minutes), the cells were switched to a C02-free 37° C. incubator. XF96 assay cartridges were calibrated according to the manufacturer's instructions. Cellular OCRs and ECARs were measured under basal conditions and following treatment with 1.5 μM oligomycin, 1.5 μM FCCP, and 40 nM rotenone, with 1 μM antimycin A (XF Cell Mito Stress kit, Agilent). OCR/ECAR ratios are calculated using the mean OCR and ECAR of 3-5 replicates for each population.

Results: Resting, distal, and proximal daughter cells were metabolically characterized (FIG. 17). The ATP production rate from oxidative phosphorylation (mitoATP) and glycolysis (glycoATP) was calculated from a Seahorse mitochondrial stress test. Mean and SD of 5 technical replicates per population is displayed in FIG. 17. The percentage above each bar quantifies the proportion of ATP produced during glycolysis. This analysis demonstrated that distal daughter cells exhibited a muted metabolic activity (as shown by the overall reduced ATP production) and increased relative ATP production from oxidative phosphorylation (as shown by the lower glyco-ATP percentage) compared to proximal daughter cells, indicating metabolic asymmetry that is established during the first cell division after CAR T cell activation.

Conclusion: The metabolic characterization of resting, distal, and proximal daughter cells revealed distinct metabolic profiles among these populations. By analyzing ATP production rates from oxidative phosphorylation (mitoATP) and glycolysis (glycoATP) using a Seahorse mitochondrial stress test, it was observed that distal daughter cells display reduced metabolic activity, as indicated by their lower overall ATP production. Moreover, distal daughter cells exhibited a higher reliance on oxidative phosphorylation for ATP generation, as evidenced by their decreased glycoATP percentage compared to proximal daughter cells. These findings demonstrate that metabolic asymmetry is established during the first cell division after CAR T cell activation, which contributes to the differential functional properties of distal and proximal daughter cells.

### Example 5: Distal Daughter Cells Transiently Exhibit Potent Cytotoxicity that Declines within Days of the First Cell Division when Compared to Proximal First Division Daughter Cells

Methods: Cytotoxicity assays were performed either on day 1-2 or day 5 after first cell division. CBG-expressing target Nalm6 cells were co-cultured with proximal, distal, resting, or donor-matched non-transduced (NTD) T-cells at indicated E:T ratios. At 4 and 20 hours after co-culture, luciferase substrate (D-luciferin potassium salt, GoldBio, final concentration 250 μg/ml) was added to each well and emitted light was measured on a luminescence plate reader (BioTek, Synergy HTX microplate reader). Percent specific lysis was calculated using the luciferase activity of 5% SDS-treated cells as maximum cell death and media alone as spontaneous cell death using the formula: Specific lysis (%)=100×[(spontaneous death data−experimental data)/(spontaneous death data−maximum death data)].

Results: Killing assays were performed on the day of the sort (day 0 after first cell division, i.e. day 3 after activation) (FIG. 18, top panel) and on day 4 (after the first cell division, i.e. day 7 after activation) (FIG. 18, bottom panel). Each dot represents the mean of a technical triplicate (FIG. 18). This experiment demonstrated that proximal and distal daughter cells exhibit similar killing directly after cytogenesis. Four days later, however, distal daughter cells demonstrated an increased decline in cytotoxicity compared to proximal daughter cells.

Conclusion: Distal daughter cells transiently exhibit potent cytotoxicity that declines within days of the first cell division when compared to proximal first division daughter cells.

### Example 6: Distal Daughter Cells Demonstrate Initial and Long-Term Leukemia Control at Suboptimal CAR T Cell Dose

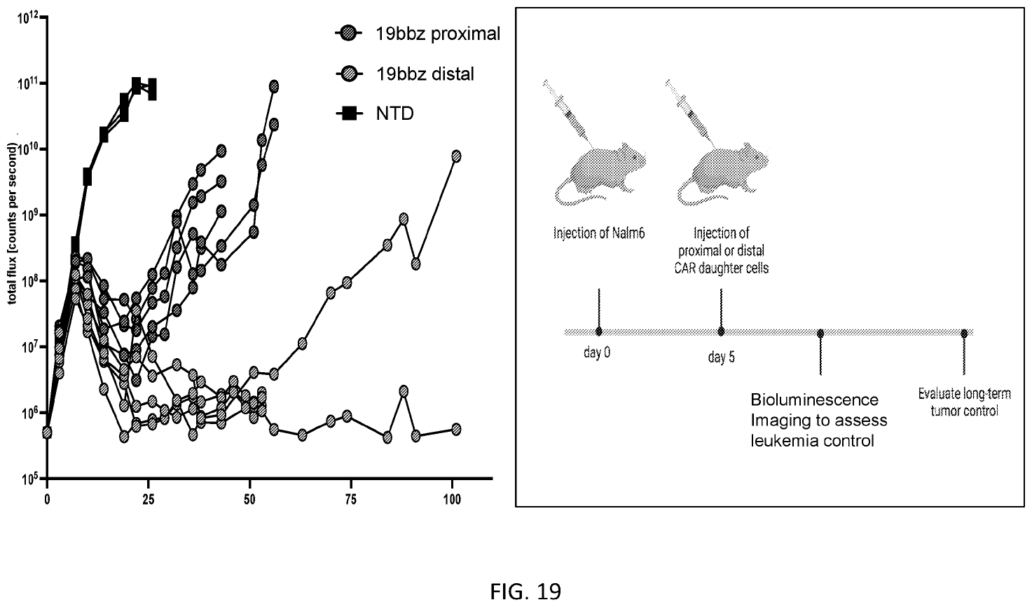

Methods: In vivo stress tests of proximal and distal daughter first division CAR daughter cells were performed. Experimental design: NSG mice were injected on day 0 with 1e6 NALM6 leukemia cells expressing click-beetle-green luciferase (FIG. 19, right panel). On day 4, proximal or distal daughter cells (2.5e5 cells per mouse) or control cells (nontransduced T cells from the same donor) were injected into NSG mice. Leukemia burden was repeatedly assessed by bioluminescence imaging. All injections were performed intravenously.

Results: Quantification of leukemia burden/rejection by in vivo bioluminescent quantification of NALM6 cells is shown in FIG. 19, left panel. NALM6 cells express click-beetle-green luciferase whose activity is used to quantify leukemia burden on indicated days. FIG. 19, right panel: Y-axis displays bioluminescent signal in photons per second, x-axis displays time in days. Each line represents one mouse. Data representative of 2 separate experiments using T cells from 2 different healthy human donors. FIG. 19, left panel depicts longevity and superior leukemia control by distal first division daughter cells.

Conclusions: Human CAR T cells undergo asymmetric cell division upon activation with distinct surface proteome, transcriptional program and metabolism of proximal and distal daughter cells. Despite their transcriptional memory precursor phenotype and in vivo longevity, first division distal daughter cells maintained increased and similar cytolytic activity as proximal daughter cells for up to 48 hours after cytogenesis. Transient effector-like state is a hallmark of distal first division daughter cells that are destined to become memory CAR T cells. These studies establish asymmetric cell division as a framework for augmenting mechanisms of CAR T cell differentiation and improving therapeutic outcomes.

### Example 7

Distal daughter CAR T cells, or a population thereof, can also be enriched by stimulating a CAR T cell with a target cell, and isolating the CAR T cell progeny for up to 7 days after stimulation. The key points of this method are: 1) the CAR does not need to have a glycine tag, 2) the target cell does not need to have a sortase-tagged antigen, 3) dye dilution is not required, and 4) target cells (live or irradiated) are incubated with CAR T cells. The CAR T cell progeny are collected 1 day, 2 days, 3 days, 4 days, 5 days, 6 days, or 7 days after stimulation yielding a population of both proximal and distal daughter cells that is relatively enriched for distal daughter cells compared to an unstimulated population or a population that is allowed to expand for longer periods of time.

### Example 4: Transient Expression of Transcription Factors in Primary Human CAR T Cells or Unmodified T Cells for CART Longevity Adjustment and Daughter Fate Induction

Another method for forcing CAR T cells to adopt a defined daughter cell fate (either proximal or distal phenotype) includes transient overexpression of a transcription factor or panel of transcription factors in primary human chimeric antigen receptor (CAR) T cells or unmodified T cells. This transient overexpression influences the first cell division after target cell encounter, causing both daughter cells to adopt the same cell fate (either proximal or distal phenotype). The methods involve the delivery of transcription factors using electroporation of protein, mRNA or circular RNA, or as mRNA or circular RNA encapsulated in lipid nanoparticles (LNPs). Transcription factors are delivered individually or as mixtures with defined ratios. This example illustrates improvement in efficacy and longevity of CAR T cell therapies and immunotherapies for treating cancer and other diseases. This example specifically includes methods for transiently overexpressing transcription factors in primary human CAR T cells or unmodified T cells to enhance the longevity of these cells.

CAR T cell therapy is a groundbreaking treatment for cancer and autoimmune patients. However, one of the limitations of this therapy is the variable persistence and functionality of CAR T cells after infusion into patients. The first cell division after target cell encounter is known to be asymmetric, with the proximal daughter cell developing into a short-lived effector cell and the distal daughter cell into a long-lived memory cell. Enhancing the longevity of both daughter cells improves the efficacy and durability of CAR T cell therapy.

In this example, methods are disclosed for transiently overexpressing transcription factors, including STAT1, STAT2, KDM5B, MXD4, MAX, KLF2, FLI1, IRF2, IRF7, IRF3, IRF9, ELK1, ELK4, ZNF358, and IKZF1, in primary human CAR T cells or unmodified T cells. The overabundance of these transcription factors prior to and during the first cell division influences the first cell division after target cell encounter, leading to both daughter cells adopting the properties of the distal daughter cell (i.e., becoming long-lived). Conversely, temporary over-expression of transcription factors associated with proximal daughter cell fate (MYC, TP73, MYBL1, SP2, YBX1, E2F2, E2F7, E2F8, NFYB) can force both daughter cells to adopt a proximal daughter cell phenotype and behavior.

Transcription factors can be delivered via electroporation of protein, mRNA or circular RNA. In another embodiment, the transcription factors are delivered as mRNA or circular RNA encapsulated in lipid nanoparticles (LNPs). In yet another embodiment, the transcription factors are delivered individually or as mixtures This allows for the optimization of the desired effect on cell longevity and functionality.

In addition to the transient overexpression of transcription factors, surface molecules (as outlined herein) are transiently overexpressed using similar methods prior to T cell infusion. This approach further enhances the properties of the CAR T cells or unmodified T cells.

Alternatively, the transcription factors are pharmacologically modified (by treating T cells with agonists or antagonists) prior to the infusion of CAR T cells. This strategy modulates the activity of the transcription factors during the first cell division and subsequently impacts the CAR T cell properties and behavior.

In one embodiment, the temporary overexpression or disruption of transcription factor RNA or DNA is achieved by transient expression of dCas9 fused to a co-activator or repressor domain or transient expression of Cas13 protein (achieved by delivery of mRNA or protein in ribonucleoprotein complexes) in conjunction with guide RNAs targeting the transcription factors above (resulting in mRNA destruction). These gene-editing techniques provide alternative methods for transiently modulating the expression of the desired transcription factors in CAR T cells or unmodified T cells during a sensitive period of fate assumption.

In conclusion, this example provides a variety of methods for transiently overexpressing or suppressing transcription factors or surface molecules, pharmacologically modifying transcription factors, or utilizing dCas9 or Cas13 for transient overexpression or disruption of transcription factors or surface proteins in primary human CAR T cells or unmodified T cells. These strategies will enhance the longevity and functionality of CAR T cells or unmodified T cells, improving the efficacy and durability of CAR T cell therapies and immunotherapies for treating cancer and other diseases.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While the embodiments provided for herein have been disclosed with reference to specific embodiments, it is apparent that other embodiments and variations of these may be devised by others skilled in the art without departing from the true spirit and scope of the present disclosure. The appended claims are intended to be construed to include all such embodiments and equivalent variations.

### ENUMERATED EMBODIMENTS

The following enumerated embodiments are provided, the numbering of which is not to be construed as designating levels of importance.

Embodiment 1 provides a method of enriching, from a population of CAR T cells, a distal first division daughter chimeric antigen receptor (CAR) T cell or population thereof, the method comprising: measuring a set of genes expressed in each of the CAR T cells in the population, wherein the genes are selected from the group consisting of CD103 (Integrin alpha E), CD45RA, CD99.1, TCRalpha/beta, CD101 (BB27), CD8, CD49a, CD7.1, CD48.1, CD52.1, CD73, CD5.1, CD3, CD224, CD45, CD47.1, CD11a, CD18, CD31, CD27.1, mouse CD49f, CD2.1, CD26, CD195 (CCR5), CD38.1, CD244 (2B4), CD29, CD314 (NKG2D), CD305 (LAIR1), CD352 (NTB-A), CD49d, CD95Fas, CD69.1, integrin beta 7, CD44.1, CD273B7 (DCPD-L2), CD45RO, CD96 (TACTILE), CD57 Recombinant, CD94, CD56, CD49b, CD226 (DNAM-1), CD278 (ICOS), CD127 (IL-7R), CLEC12A.1, CD39, CD161, HLA-DR, CD81 (TAPA-1), HLA-E.1, HLA-ABC, CD66ace, and CD28.1, wherein when expression of at least one of these genes is increased in the CAR T cell relative to a control, the cell is identified as a distal daughter CAR T cell and is collected.

Embodiment 2 provides the method of embodiment 1, wherein the control is selected from the group consisting of a proximal daughter CAR T cell, a resting CAR T cell, and a non-enriched CAR T cell population.

Embodiment 3 provides a composition comprising a population of distal daughter CAR T cells isolated by the method of embodiment 1 or 2.

Embodiment 4 provides the composition of embodiment 3, wherein the population comprises over 50%, 60%, 70%, 80%, 90%, or 100% distal daughter CAR T cells.

Embodiment 5 provides a method of enriching, from a population of CAR T cells, a proximal first division daughter CAR T cell or population thereof, the method comprising: measuring a set of genes expressed in each of the CAR T cells in the population, wherein the genes are selected from the group consisting of CD30, CD71, CD25, CD106, CD117 (c-kit), CD88 (C5aR), CDllc, CD155 (PVR), CD223 (LAG-3), CD146, CD163.1, CD19.1, CD194 (CCR4), CD23, Notch1, CD105, CD169 (Sialoadhesin Siglec-1), CD83.1, aCD207, CD137 (4-1BB), TCRdeltagamma, CD134 (OX40), B7-H4, CD324E (Cadherin), CD122 (IL-2R beta), CD10, CD206 (MMR), CD178 (Fas-L), CD82.1, CD141 (Thrombomodulin), CD80.1, LOX-1, CD1a, IgGFc, CD123, TCRValpha24, CD209 (DC-SIGN), CD272 (BTLA), CD304 (Neuropilin-1), CD85 (jILT2), CD252 (OX40L), CD303 (BDCA2), IgM, TSLPRTSLP-R, CD98, CD34.1, CD20, CD235ab, CD62L, CD144VE (Cadherin), CD307d (FcRL4), CD197 (CCR7), CD201 (EPCR), CD54, CX3CR1.1, CD360 (IL-21R), CD140b (PDGFRbeta), CD112 (Nectin-2), CD124 (IL-4Ralpha), CD257 (BAFFBLYS), CD335 (NKp46), CD152 (CTLA-4), EGFR.1, GARPLRRC32, CD62E, CD269 (BCMA), CD158 (KIR2DL1S1S3S5), Podoplanin, XCR1.1, CD70.1, CD254 (TRANCERANKL), Podocalyxin, CD158f (KIR2DL5), CD274 (B7-H1 PD-L1), CD66b, CD21, CD307 (eFcRL5), CD268 (BAFF-R), CD1 54, CD137L (4-1BB Ligand), CD119 (IFN gamma R alpha chain), CD1d, CD370 (CLEC9ADNGR1), CD267 (TACI), CD107a (LAMP-1), CD24.1, CD13, TCRVgamma9, CD357 (GITR), TCRVdelta2, Notch3, CD40.1, CD326 (Ep-CAM), CD204, Fc epsilon RI alpha, CD294 (CRTH2), CD158el (KIR3DL1, NKB1), CD150 (SLAM), CD14.1, Ig light chain kappa, LIPSTIC1, CD184 (CXCR4), CD196 (CCR6), CD79b (IgB), CD16, DR3TRAMP, CD319 (CRACC), CD258 (LIGHT), CD32, TCRVbeta131, CD275 (B7-H2, ICOSL), CD45R/B220, CD279, CD35, CD42b, CD366 (Tim-3), CD336 (NKp44), CD140a (PDGFRalpha), IgD, CD62P (P-Selectin), CD1c, CD41, CD11b, CD185 (CXCR5), CD22.1, CD328 (Siglec-7), CD325 (N-Cadherin), CD86.1, CD36.1, CD158b (KIR2DL2/L3, NKAT2, KLRG1, MAFA), Ig light chain lambda, GPR56, TIGIT/VSTM3, CD337 (NKp30), CD64, CD33.1, TCRValpha72, CD270 (HVEMTR2), CD4.1, HLAA2, CD183 (CXCR3), and CD58 (LFA-3), wherein when expression of at least one of these genes is increased relative to a control, the cell is identified as a proximal daughter CAR T cell and is collected.

Embodiment 6 provides the method of embodiment 5, wherein the control is selected from the group consisting of a distal daughter CAR T cell, a resting CAR T cell, or a non-enriched CAR T cell population.

Embodiment 7 provides a composition comprising a population of proximal daughter CAR T cells isolated by the method of embodiment 5 or 6.

Embodiment 8 provides the composition of embodiment 7, wherein the population comprises over 50%, 60%, 70%, 80%, 90%, or 100% proximal daughter CAR T cells.

Embodiment 9 provides a method of enriching, from a population of CAR T cells, a distal first division daughter CAR T cell or population thereof, the method comprising: i) pre-loading a LPETG peptide comprising a detectable label onto a Sortase A (SrtA) molecule, ii) fusing the SrtA to a target protein on a target cell, iii) labeling with a dye, a CAR T cell comprising at least one N-terminal glycine on the CAR, iv) incubating the target cell with the labeled CAR T cell, v) assessing CAR T cell division by dye dilution, indicating daughter cell formation, and vi) measuring the detectable label on the daughter cells following the first cell division of the CAR T cell, wherein when the detectable label is present, the cell is a proximal first division daughter cell, and wherein when the detectable label is absent, the cell is a distal first division daughter cell, and collecting the distal daughter CAR T cell.

Embodiment 10 provides the method of embodiment 9, wherein the target protein is a tumor associated antigen (TAA).

Embodiment 11 provides the method of embodiment 9 or 10, wherein the target cell is selected from the group consisting of a cancer cell, an autoimmune cell, an alloimmune immune cell, an infected cell, and a diseased cell in a fibrotic disease.

Embodiment 12 provides the method of any one of claims 9-11, wherein the detectable label is a biotin or a fluorophore.

Embodiment 13 provides the method of any one of embodiments 9-12, wherein the dye is selected from the group consisting of CFSE, CellTrace™ Violet, CellTrace™ Red, and CellTrace™ Yellow.

Embodiment 14 provides a composition comprising a population of distal daughter CAR T cells isolated by the method of any of embodiments 9-13.

Embodiment 15 provides the composition of embodiment 14, wherein the population comprises over 50%, 60%, 70%, 80%, 90%, or 100% distal daughter CAR T cells.

Embodiment 16 provides the method of any of the preceding embodiments, further comprising allowing the daughter cell to undergo a second division, and isolating and/or collecting the distal second division daughter cell.

Embodiment 17 provides the method of embodiment 16, further comprising allowing the daughter cell to undergo a third division, and isolating and/or collecting the distal third division daughter cell.

Embodiment 18 provides a composition comprising a population of distal second division daughter cells isolated and/or collected by the method of embodiment 16.

Embodiment 19 provides a composition comprising a population of distal third division daughter cells isolated and/or collected by the method of embodiment 18.

Embodiment 20 provides a composition comprising a population of first and/or second, and/or third division daughter cells isolated and/or collected by any of the preceding embodiments.

Embodiment 21 provides a method of inducing a T cell to adopt a distal first division daughter cell phenotype, the method comprising: introducing at least one transcription factor into a primary T cell such that the transcription factor is transiently overexpressed, wherein the transcription factor is selected from the group consisting of STAT1, STAT2, KDM5B, MXD4, MAX, KLF2, FLI1, IRF2, IRF7, IRF3, IRF9, ELK1, ELK4, ZNF358, and IKZF1.

Embodiment 22 provides the method of embodiment 21, further comprising isolating and/or collecting the distal first division daughter cell, or population thereof.

Embodiment 23 provides the method of embodiment 21, wherein the T cell is a chimeric antigen receptor (CAR) T cell.

Embodiment 24 provides the method of embodiment 23, wherein the method improves the efficacy and longevity of the CAR T cell.

Embodiment 25 provides the method of embodiment 23, further comprising isolating the distal first division daughter CAR T cell, or population thereof.

Embodiment 26 provides a method of inducing a T cell to adopt a proximal first division daughter cell phenotype, the method comprising: introducing at least one transcription factor into a primary T cell such that the transcription factor is transiently overexpressed, wherein the transcription factor is selected from the group consisting of MYC, TP73, MYBL1, SP2, YBX1, E2F2, E2F7, E2F8, and NFYB.

Embodiment 27 provides the method of embodiment 26, further comprising isolating and/or collecting the proximal first division daughter cell, or population thereof.

Embodiment 28 provides the method of embodiment 26, wherein the T cell is a chimeric antigen receptor (CAR) T cell.

Embodiment 29 provides the method of embodiment 28, further comprising isolating the proximal first division daughter CAR T cell, or population thereof.

Embodiment 30 provides the method of any one of embodiments 21-29, wherein the transcription factor is introduced via a method selected from the group consisting of electroporation of the protein, mRNA, or circular RNA form of the transcription factor.

Embodiment 31 provides the method of any one of embodiments 21-29, wherein the transcription factor is introduced as an mRNA or circular RNA encapsulated in a lipid nanoparticle (LNP).

Embodiment 32 provides the method of any one of embodiments 21-29, wherein a single transcription factor is introduced into the cell.

Embodiment 33 provides the embodiment of any one of claims 21-29, wherein a plurality of transcription factors are introduced into the cell.

Embodiment 34 provides a method of enriching for distal daughter CAR T cells in a population of CAR T cells, the method comprising stimulating a CAR T cell with a target cell and collecting the CAR T cell progeny 1 day, 2 days, 3 days, 4 days, 5 days, 6 days, or 7 days after stimulation, wherein the progeny is thereby enriched for distal daughter CAR T cells.

Embodiment 35 provides a composition comprising a population of distal daughter CAR T cells isolated by the method of embodiment 34.

Embodiment 36 provides the composition of embodiment 25, wherein the population comprises over 50%, 60%, 70%, 80%, 90%, or 100% distal daughter CAR T cells.

Embodiment 37 provides a method of treating a disease or disorder, the method comprising administering to a subject in need thereof, the composition of any one of embodiments 3, 4, 14, 15, 18, 19, 20, 35, or 36.

1. A method of enriching, from a population of CAR T cells, a distal first division daughter chimeric antigen receptor (CAR) T cell or population thereof, the method comprising:
measuring a set of genes expressed in each of the CAR T cells in the population,
wherein the genes are selected from the group consisting of CD103 (Integrin alpha E), CD45RA, CD99.1, TCRalpha/beta, CD101 (BB27), CD8, CD49a, CD7.1, CD48.1, CD52.1, CD73, CD5.1, CD3, CD224, CD45, CD47.1, CD11a, CD18, CD31, CD27.1, mouse CD49f, CD2.1, CD26, CD195 (CCR5), CD38.1, CD244 (2B4), CD29, CD314 (NKG2D), CD305 (LAIR1), CD352 (NTB-A), CD49d, CD95Fas, CD69.1, integrin beta 7, CD44.1, CD273B7 (DCPD-L2), CD45RO, CD96 (TACTILE), CD57 Recombinant, CD94, CD56, CD49b, CD226 (DNAM-1), CD278 (ICOS), CD127 (IL-7R), CLEC12A.1, CD39, CD161, HLA-DR, CD81 (TAPA-1), HLA-E.1, HLA-ABC, CD66ace, and CD28.1,
wherein when expression of at least one of these genes is increased in the CAR T cell relative to a control, the cell is identified as a distal daughter CAR T cell and is collected.

2. The method of claim 1, wherein the control is selected from the group consisting of a proximal daughter CAR T cell, a resting CAR T cell, and a non-enriched CAR T cell population.

3. A composition comprising a population of distal daughter CAR T cells isolated by the method of claim 1.

4. The composition of claim 3, wherein the population comprises over 50%, 60%, 70%, 80%, 90%, or 100% distal daughter CAR T cells.

5. A method of enriching, from a population of CAR T cells, a proximal first division daughter CAR T cell or population thereof, the method comprising: measuring a set of genes expressed in each of the CAR T cells in the population,
wherein the genes are selected from the group consisting of CD30, CD71, CD25, CD106, CD117 (c-kit), CD88 (C5aR), CDllc, CD155 (PVR), CD223 (LAG-3), CD146, CD163.1, CD19.1, CD194 (CCR4), CD23, Notch1, CD105, CD169 (Sialoadhesin Siglec-1), CD83.1, aCD207, CD137 (4-1BB), TCRdeltagamma, CD134 (OX40), B7-H4, CD324E (Cadherin), CD122 (IL-2R beta), CD10, CD206 (MMR), CD178 (Fas-L), CD82.1, CD141 (Thrombomodulin), CD80.1, LOX-1, CD1a, IgGFc, CD123, TCRValpha24, CD209 (DC-SIGN), CD272 (BTLA), CD304 (Neuropilin-1), CD85 (jILT2), CD252 (OX40L), CD303 (BDCA2), IgM, TSLPRTSLP-R, CD98, CD34.1, CD20, CD235ab, CD62L, CD144VE (Cadherin), CD307d (FcRL4), CD197 (CCR7), CD201 (EPCR), CD54, CX3CR1.1, CD360 (IL-21R), CD140b (PDGFRbeta), CD112 (Nectin-2), CD124 (IL-4Ralpha), CD257 (BAFFBLYS), CD335 (NKp46), CD152 (CTLA-4), EGFR.1, GARPLRRC32, CD62E, CD269 (BCMA), CD158 (KIR2DLISIS3S5), Podoplanin, XCR1.1, CD70.1, CD254 (TRANCERANKL), Podocalyxin, CD158f (KIR2DL5), CD274 (B7-H1 PD-L1), CD66b, CD21, CD307 (eFcRL5), CD268 (BAFF-R), CD154, CD137L (4-1BB Ligand), CD119 (IFN gamma R alpha chain), CD1d, CD370 (CLEC9ADNGR1), CD267 (TACI), CD107a (LAMP-1), CD24.1, CD13, TCRVgamma9, CD357 (GITR), TCRVdelta2, Notch3, CD40.1, CD326 (Ep-CAM), CD204, Fc epsilon RI alpha, CD294 (CRTH2), CD158el (KIR3DL1, NKB1), CD150 (SLAM), CD14.1, Ig light chain kappa, LIPSTIC1, CD184 (CXCR4), CD196 (CCR6), CD79b (IgB), CD16, DR3TRAMP, CD319 (CRACC), CD258 (LIGHT), CD32, TCRVbeta131, CD275 (B7-H2, ICOSL), CD45R/B220, CD279, CD35, CD42b, CD366 (Tim-3), CD336 (NKp44), CD140a (PDGFRalpha), IgD, CD62P (P-Selectin), CD1c, CD41, CD11b, CD185 (CXCR5), CD22.1, CD328 (Siglec-7), CD325 (N-Cadherin), CD86.1, CD36.1, CD158b (KIR2DL2/L3, NKAT2, KLRG1, MAFA), Ig light chain lambda, GPR56, TIGIT/VSTM3, CD337 (NKp30), CD64, CD33.1, TCRValpha72, CD270 (HVEMTR2), CD4.1, HLAA2, CD183 (CXCR3), and CD58 (LFA-3),
wherein when expression of at least one of these genes is increased relative to a control, the cell is identified as a proximal daughter CAR T cell and is collected.

6. The method of claim 5, wherein the control is selected from the group consisting of a distal daughter CAR T cell, a resting CAR T cell, or a non-enriched CAR T cell population.

7. A composition comprising a population of proximal daughter CAR T cells isolated by the method of claim 5.

8. The composition of claim 7, wherein the population comprises over 50%, 60%, 70%, 80%, 90%, or 100% proximal daughter CAR T cells.

9. A method of enriching, from a population of CAR T cells, a distal first division daughter CAR T cell or population thereof, the method comprising:
i) pre-loading a LPETG peptide comprising a detectable label onto a Sortase A (SrtA) molecule,
ii) fusing the SrtA to a target protein on a target cell,
iii) labeling with a dye, a CAR T cell comprising at least one N-terminal glycine on the CAR,
iv) incubating the target cell with the labeled CAR T cell,
v) assessing CAR T cell division by dye dilution, indicating daughter cell formation, and
vi) measuring the detectable label on the daughter cells following the first cell division of the CAR T cell,
wherein when the detectable label is present, the cell is a proximal first division daughter cell, and
wherein when the detectable label is absent, the cell is a distal first division daughter cell, and
collecting the distal daughter CAR T cell.

10. The method of claim 9, wherein the target protein is a tumor associated antigen (TAA).

11. The method of claim 9, wherein the target cell is selected from the group consisting of a cancer cell, an autoimmune cell, an alloimmune immune cell, an infected cell, and a diseased cell in a fibrotic disease.

12. The method of claim 9, wherein the detectable label is a biotin or a fluorophore.

13. The method of claim 9, wherein the dye is selected from the group consisting of CFSE, CellTrace™ Violet, CellTrace™ Red, and CellTrace™ Yellow.

14. A composition comprising a population of distal daughter CAR T cells isolated by the method of claim 9.

15. The composition of claim 14, wherein the population comprises over 50%, 60%, 70%, 80%, 90%, or 100% distal daughter CAR T cells.

16. The method of claim 1, further comprising allowing the daughter cell to undergo a second division, and isolating and/or collecting the distal second division daughter cell.

17. The method of claim 16, further comprising allowing the daughter cell to undergo a third division, and isolating and/or collecting the distal third division daughter cell.

18. A composition comprising a population of distal second division daughter cells isolated and/or collected by the method of claim 16.

19. A composition comprising a population of distal third division daughter cells isolated and/or collected by the method of claim 18.

20. A composition comprising a population of first and/or second, and/or third division daughter cells isolated and/or collected by the method of claim 1.

21. A method of inducing a T cell to adopt a distal first division daughter cell phenotype, the method comprising:
introducing at least one transcription factor into a primary T cell such that the transcription factor is transiently overexpressed,
wherein the transcription factor is selected from the group consisting of STAT1, STAT2, KDM5B, MXD4, MAX, KLF2, FLI1, IRF2, IRF7, IRF3, IRF9, ELK1, ELK4, ZNF358, and IKZF1.

22. The method of claim 21, further comprising isolating and/or collecting the distal first division daughter cell, or population thereof.

23. The method of claim 21, wherein the T cell is a chimeric antigen receptor (CAR) T cell.

24. The method of claim 23, wherein the method improves the efficacy and longevity of the CAR T cell.

25. The method of claim 23, further comprising isolating the distal first division daughter CAR T cell, or population thereof.

26. A method of inducing a T cell to adopt a proximal first division daughter cell phenotype, the method comprising:
introducing at least one transcription factor into a primary T cell such that the transcription factor is transiently overexpressed,
wherein the transcription factor is selected from the group consisting of MYC, TP73, MYBL1, SP2, YBX1, E2F2, E2F7, E2F8, and NFYB.

27. The method of claim 26, further comprising isolating and/or collecting the proximal first division daughter cell, or population thereof.

28. The method of claim 26, wherein the T cell is a chimeric antigen receptor (CAR) T cell.

29. The method of claim 28, further comprising isolating the proximal first division daughter CAR T cell, or population thereof.

30. The method of claim 21, wherein the transcription factor is introduced via a method selected from the group consisting of electroporation of the protein, mRNA, or circular RNA form of the transcription factor.

31. The method of claim 21, wherein the transcription factor is introduced as an mRNA or circular RNA encapsulated in a lipid nanoparticle (LNP).

32. The method of claim 21, wherein a single transcription factor is introduced into the cell.

33. The method of claim 21, wherein a plurality of transcription factors are introduced into the cell.

34. A method of enriching for distal daughter CAR T cells in a population of CAR T cells, the method comprising stimulating a CAR T cell with a target cell and collecting the CAR T cell progeny 1 day, 2 days, 3 days, 4 days, 5 days, 6 days, or 7 days after stimulation, wherein the progeny is thereby enriched for distal daughter CAR T cells.

35. A composition comprising a population of distal daughter CAR T cells isolated by the method of claim 34.

36. The composition of claim 25, wherein the population comprises over 50%, 60%, 70%, 80%, 90%, or 100% distal daughter CAR T cells.

37. A method of treating a disease or disorder, the method comprising administering to a subject in need thereof, the composition of claim 3.


SEQUENCE LISTING (46 sequences)

SEQ ID NO: 1 - protein, 521 aa, synthetic construct
MEFGLSWLFLVAILKGVQCGSDYKDDDDKGSGLEQAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRNVKPTDVGVLDEQKGKDKQLTLITCDDYNEKTGVWETRKIFVATEVKTGSTSGSGKPGSGEGSTKGSTSGSGKPGSGDGSTKGTRGSPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVL

SEQ ID NO: 2 - protein, 917 aa, synthetic construct
MEFGLSWLFLVAILKGVQCGSDYKDDDDKGSGLEQAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRNVKPTDVGVLDEQKGKDKQLTLITCDDYNEKTGVWETRKIFVATEVKTGSTSGSGKPGSGEGSTKGSTSGSGKPGSGDGSTKGTRGSKSSNLEGRTKSVTRPTGSSAVITCDLPVENAVYTHWYLHQEGKAPQRLLYYDSYNSRVVLESGISREKYHTYASTGKSLKFILENLIERDSGVYYCATWKDYYKKLFGSGTTLVVTDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKDSYSKDANDVTTVDPKYNYSKDANDVITMDPKDNWSKDANDTLLLQLTNTSAYYMYLLLLLKSVVYFAIITCCLLGRTAFCCNGEKSGSGATNFSLLKQAGDVEENPGPMLFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGTGVRGLQDTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFLDHVKPKETENTKQPSKSCHKPKAIVHTEKVNMMSLTVLGLRMLFAKTVAVNFLLTAKLFFL

SEQ ID NO: 3 - protein, 491 aa, synthetic construct
MEFGLSWLFLVAILKGVQCGGGGGGSDIQMTQTTSILSASLGDRVTITCRASQDISNYLNWYQQNPDGTVKLLIYYTSRLHSGVPSRFSGSGSGTEYSLTIKNLEQEDIATYFCQQGNMVPFTFGSGTKLEIKGGGGSGGGGSGGGGSQVQLKESGPGLVAPSQSLSITCTVSGFSLTSYGVHWVRQPPGKGLEWLGVIWASGTTDYNSALMSRLTISKDNSKSQVFLRMNSLQTDDTAMYYCARETTASFGYWGLGTLVTVSAASTTTPAPRPPTPAPTIASQPLSLRPEACRPAAGGAVHTRGLDFACDSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 495 aa, synthetic construct
MEFGLSWLFLVAILKGVQCGGGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTTTPAPRPPTPAPTIASQPLSLRPEACRPAAGGAVHTRGLDFACDSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 2571 bp, synthetic construct
  source 1..2571: mol_type = other DNA
atggagtttgggctgagctggctttttcttgtggctattttaaaaggtgtccagtgcggatctgactataaagatgatgatgataaagggagcggcctggagcaagccaaaccccaaattcccaaggataagagtaaggttgcaggttatatagagatacccgatgcagacataaaggagccggtatatcctgggcccgccacgagcgaacagctcaacaggggagtatcatttgcagaagagaacgagtcccttgatgaccaaaacataagtatcgcaggacataccttcatcgaccgcccgaattatcagttcactaacctcaaagccgcgaaaaaggggtccatggtatatttcaaagtaggaaatgagactagaaaatataaaatgacttctattcgaaatgtcaagcccacggacgttggtgtactcgatgagcaaaaaggcaaagacaaacaactcacgctcatcacttgcgacgattacaatgaaaagacaggcgtctgggaaacacgcaaaatcttcgtggcaacagaggtcaagacgggtagcaccagtggtagtggcaaacctggttctggtgaaggaagcaccaaagggagcactagcggcagcggaaagccaggcagtggggatggaagtacgaaaggaactcgcggatccaagtcaagtaatttggagggtaggacaaagagtgtaaccaggccaaccggaagttctgccgtgattacatgtgaccttccggtagaaaatgctgtttatacacattggtatctgcaccaagagggtaaagcaccgcaaaggctcttgtactatgacagttacaactctagggtagttttggagagtgggattagtagggagaaatatcacacatacgcatctacgggcaaatctctgaaatttattctcgagaatcttatagagcgagactcaggggtttattattgcgccacctggaaagactattataagaaattgtttggatctggcacgacgcttgtagtgacggataagcaacttgatgccgatgtttcaccaaaacccaccattttcttgccatctatcgcggagaccaaactgcagaaggcaggtacatatctttgtctcttggaaaaattctttccggacataataaagatccactggcaggagaagaagagcaacactatccttgggtcacaagaaggcaatacgatgaaaacgaatgatacatatatgaagttttcttggctgaccgttccagaggaaagcttggataaggaacacaggtgcatagtaagacatgagaataacaagaacgggatcgaccaagaaatcatattcccaccgattaagactgatgtgactacggtggaccccaaggactcctactccaaagatgcgaacgacgtaactacggtagaccccaaatataactacagtaaagacgcaaatgacgtgataaccatggacccaaaggacaactggagtaaggatgctaacgatacgctgctgcttcagcttacgaatactagtgcttattacatgtacctgctgcttctgctcaagagtgtagtctattttgccatcattacgtgttgccttcttggacgcacggccttttgctgcaacggtgagaaatccgggtcaggtgctactaacttcagtcttctgaaacaagcaggagatgtggaagaaaaccctggtcctatgttgttttcaagcctgctctgcgtattcgtggctttttcctactctggttcttccgtcgctcagaaggtcacccaagcgcagagtagcgtatccatgcctgttcggaaagcggtgacactcaactgtctctacgaaacgagctggtggtcctattatatcttctggtacaagcaactccctagcaaggaaatgatattcttgatacgccaaggctccgatgagcagaatgcaaaatccggtcgctatagcgtaaattttaagaaggccgcgaaaagcgttgccctcaccatcagtgcattgcagctcgaagacagcgcaaaatacttctgtgcactcggaactggggtgagaggtttgcaggacactgataaacttatatttgggaaaggcactcgagtcactgtcgagccccgctcacagccacacaccaagccctccgtatttgttatgaaaaacggcaccaatgtagcatgcctcgtgaaagagttctatcccaaagacataaggatcaatctcgtgtcttcaaaaaaaatcacagaattcgatcccgcaatcgtcataagcccgtcaggtaagtacaatgctgtgaaactgggaaagtacgaggattccaactctgtgacttgttcagtgcagcatgataacaagaccgttcattcaaccgactttgaagtcaaaacagatagtacagaccacgtaaagccaaaagagactgaaaatacaaagcaaccttctaagtcatgccacaagcccaaggctatagtgcatacagagaaggtcaacatgatgagcttgacggttctcggcctccgaatgctttttgccaagactgtcgccgtaaactttctcctcacagcaaaactgttttttctctga

SEQ ID NO: 6 - DNA, 1569 bp, synthetic construct
  source 1..1569: mol_type = other DNA
atggagtttgggctgagctggctttttcttgtggctattttaaaaggtgtccagtgcggatctgactataaagatgatgatgataaagggagcggcctggagcaagccaaaccccaaattcccaaggataagagtaaggttgcaggttatatagagatacccgatgcagacataaaggagccggtatatcctgggcccgccacgagcgaacagctcaacaggggagtatcatttgcagaagagaacgagtcccttgatgaccaaaacataagtatcgcaggacataccttcatcgaccgcccgaattatcagttcactaacctcaaagccgcgaaaaaggggtccatggtatatttcaaagtaggaaatgagactagaaaatataaaatgacttctattcgaaatgtcaagcccacggacgttggtgtactcgatgagcaaaaaggcaaagacaaacaactcacgctcatcacttgcgacgattacaatgaaaagacaggcgtctgggaaacacgcaaaatcttcgtggcaacagaggtcaagacgggtagcaccagtggtagtggcaaacctggttctggtgaaggaagcaccaaagggagcactagcggcagcggaaagccaggcagtggggatggaagtacgaaaggaactcgcggatcccccgaggaacccctcgtagttaaggtagaggagggtgacaacgctgtccttcaatgcctgaaaggcacatctgacggccctacacagcaattgacctggtctcgcgaatcaccgcttaagccgttcctgaaactctctctcgggctcccaggacttggcatccacatgcgcccccttgccatttggcttttcatattcaatgtatctcagcaaatgggcggcttctatctgtgtcagccggggccgccctccgaaaaagcctggcagccgggctggaccgttaatgttgagggctcaggcgagttgttccgctggaacgtaagtgatttgggtggtctgggttgtgggctgaaaaacagaagctccgaagggccctccagcccttctgggaaactgatgagccctaagttgtacgtctgggcgaaggatcggcccgagatttgggagggcgagccaccatgtctgcccccgcgggattcactgaaccagtccctttcacaagacctcacaatggcgcccggctccacgctctggttgagttgtggtgtgcctcctgactccgtcagcaggggacccctttcatggacacatgttcaccctaaaggaccgaaaagcctcctctcactcgagcttaaggatgatcgacctgccagagacatgtgggtcatggagaccgggctgctccttccgagagcgacggctcaggacgcggggaagtattattgtcaccgcggaaatctcaccatgagtttccatttggaaataaccgctcgcccagtcctctggcattggctgcttcggactggagggtggaaagtaagcgccgttaccctggcatacctcatcttttgcttgtgctccctggtcggcatactgcaccttcaacgcgcgctcgttctctgataa

SEQ ID NO: 7 - DNA, 1476 bp, synthetic construct
  source 1..1476: mol_type = other DNA
atggagtttgggctgagctggctttttcttgtggctattttaaaaggtgtccagtgcggaggcggaggaggcggatccgatatccaaatgacgcaaactacgtctatcctgtctgcctccttgggcgaccgggtgacgattacgtgccgggcatctcaagatattagtaactatcttaactggtatcaacagaaccctgacggaacggtgaaattgctcatttactacacttctagacttcatagtggcgtgccgtccagattttccggaagtgggtcaggtacagaatactcactgactattaagaacctggaacaagaggacatagccacatatttttgtcagcaaggtaatatggtcccttttaccttcggcagtggtactaagctcgaaataaagggaggagggggtagcggaggtggcggctcaggcggcggcggcagtcaggttcaacttaaggaatccggtcccggtcttgtagcgccaagtcagtctctctctatcacttgtacggtatccgggttctcccttacgtcctacggggtacactgggttcgacaaccacccggaaagggcctggagtggttgggcgtcatatgggcaagcggaactacggattataactctgcccttatgtctcgcctcaccatttctaaagataatagtaaaagccaggtttttcttcgcatgaactctctccaaactgatgacacagcaatgtactactgcgccagggagactacagcgagtttcggttattggggcttgggcacactggtcacagtttcagcagctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattccggaatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaagatga

SEQ ID NO: 8 - DNA, 1488 bp, synthetic construct
  source 1..1488: mol_type = other DNA
atggagtttgggctgagctggctttttcttgtggctattttaaaaggtgtccagtgcggaggcggaggaggcggatccgacattcaaatgactcaaacaacatcctcactttctgcctcactcggagatcgcgtcactatatcatgcagggcttcccaagatattagcaagtatctgaactggtatcagcaaaaaccggatggaacggttaaactcttgatttaccacacgtcccgcctccattcaggcgttcccagcagatttagcggctcaggctctggtaccgattattcacttactatatccaatttggaacaggaagacatagctacctatttctgtcaacaagggaatacgcttccgtacacatttggtgggggtactaagctggagattacaggcggaggaggttctggcggcggagggtcagggggcggtggttcagaggtgaagctgcaagagtcaggtccgggattggtagctccgagtcaaagcttgtccgtgacatgcaccgtgtcaggtgtcagcctcccagactacggtgtgtcctggatcagacaacctcctcggaaggggttggagtggctcggtgtgatttgggggtcagagacaacctactacaactcagcactcaagagtcgcctgacaattattaaagacaacagcaaaagtcaagtcttcctcaagatgaacagcctgcagactgacgataccgccatatattattgtgcgaagcactattactacggaggttcctatgcaatggattattgggggcaagggacgagtgtaacagtcagcagtgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgattccggaatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaagatga

SEQ ID NO: 9 - protein, 19 aa, synthetic construct
MEFGLSWLFLVAILKGVQC

SEQ ID NO: 10 - protein, 8 aa, synthetic construct
DYKDDDDK

SEQ ID NO: 11 - protein, 300 aa, synthetic construct
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVL

SEQ ID NO: 12 - protein, 4 aa, synthetic construct
GSGL

SEQ ID NO: 13 - protein, 38 aa, synthetic construct
TGSTSGSGKPGSGEGSTKGSTSGSGKPGSGDGSTKGTR

SEQ ID NO: 14 - DNA, 45 bp, synthetic construct
  source 1..45: mol_type = other DNA
ggtggcggtggctcgggcggtggtgggtcgggtggcggcggatct

SEQ ID NO: 15 - protein, 148 aa, synthetic construct
EQAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRNVKPTDVGVLDEQKGKDKQLTLITCDDYNEKTGVWETRKIFVATEVK

SEQ ID NO: 16 - protein, 21 aa, synthetic construct
SAVTLAYLIFCLCSLVGILHL

SEQ ID NO: 17 - protein, 321 aa, synthetic construct
KSSNLEGRTKSVTRPTGSSAVITCDLPVENAVYTHWYLHQEGKAPQRLLYYDSYNSRVVLESGISREKYHTYASTGKSLKFILENLIERDSGVYYCATWKDYYKKLFGSGTTLVVTDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKDSYSKDANDVTTVDPKYNYSKDANDVITMDPKDNWSKDANDTLLLQLTNTSAYYMYLLLLLKSVVYFAIITCCLLGRTAFCCNGEKS

SEQ ID NO: 18 - protein, 353 aa, synthetic construct
MLFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGTGVRGLQDTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFLDHVKPKETENTKQPSKSCHKPKAIVHTEKVNMMSLTVLGLRMLFAKTVAVNFLLTAKLFFL

SEQ ID NO: 19 - protein, 22 aa, synthetic construct
GSGATNFSLLKQAGDVEENPGP

SEQ ID NO: 20 - protein, 238 aa, synthetic construct
DIQMTQTTSILSASLGDRVTITCRASQDISNYLNWYQQNPDGTVKLLIYYTSRLHSGVPSRFSGSGSGTEYSLTIKNLEQEDIATYFCQQGNMVPFTFGSGTKLEIKGGGGSGGGGSGGGGSQVQLKESGPGLVAPSQSLSITCTVSGFSLTSYGVHWVRQPPGKGLEWLGVIWASGTTDYNSALMSRLTISKDNSKSQVFLRMNSLQTDDTAMYYCARETTASFGYWGLGTLVTVSA

SEQ ID NO: 21 - protein, 227 aa, synthetic construct
ASTTTPAPRPPTPAPTIASQPLSLRPEACRPAAGGAVHTRGLDFACDSGIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 5 aa, synthetic construct
GGGGG

SEQ ID NO: 23 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 24 - protein, 5 aa, synthetic construct
  REPEAT 1..5: note = repeat n times, where n is an integer of 1
GSGGS

SEQ ID NO: 25 - protein, 4 aa, synthetic construct
  REPEAT 1..4: note = repeat n times, where n is an integer of 1
GGGS

SEQ ID NO: 26 - protein, 5 aa, synthetic construct
  REPEAT 1..5: note = repeat n times, where n is an integer of 1
GGGGS

SEQ ID NO: 27 - protein, 4 aa, synthetic construct
GGSG

SEQ ID NO: 28 - protein, 5 aa, synthetic construct
GGSGG

SEQ ID NO: 29 - protein, 5 aa, synthetic construct
GSGSG

SEQ ID NO: 30 - protein, 5 aa, synthetic construct
GSGGG

SEQ ID NO: 31 - protein, 5 aa, synthetic construct
GGGSG

SEQ ID NO: 32 - protein, 5 aa, synthetic construct
GSSSG

SEQ ID NO: 33 - protein, 5 aa, synthetic construct
GGGGS

SEQ ID NO: 34 - protein, 15 aa, synthetic construct
GGGGSGGGGSGGGGS

SEQ ID NO: 35 - protein, 5 aa, synthetic construct
DKTHT

SEQ ID NO: 36 - protein, 4 aa, synthetic construct
CPPC

SEQ ID NO: 37 - protein, 15 aa, synthetic construct
CPEPKSCDTPPPCPR

SEQ ID NO: 38 - protein, 12 aa, synthetic construct
ELKTPLGDTTHT

SEQ ID NO: 39 - protein, 10 aa, synthetic construct
KSCDKTHTCP

SEQ ID NO: 40 - protein, 7 aa, synthetic construct
KCCVDCP

SEQ ID NO: 41 - protein, 7 aa, synthetic construct
KYGPPCP

SEQ ID NO: 42 - protein, 15 aa, synthetic construct
EPKSCDKTHTCPPCP

SEQ ID NO: 43 - protein, 12 aa, synthetic construct
ERKCCVECPPCP

SEQ ID NO: 44 - protein, 17 aa, synthetic construct
ELKTPLGDTTHTCPRCP

SEQ ID NO: 45 - protein, 12 aa, synthetic construct
SPNMVPHAHHAQ

SEQ ID NO: 46 - protein, 15 aa, synthetic construct
EPKSCDKTYTCPPCP